SEQUENCE LISTING (11 sequences)

SEQ ID NO: 1 - protein, 143 aa, synthetic construct
  REGION 1..143: note = CRABP2-H6
PNFSGNWKIIRSENFEELLKVLGVNVMLRKIAVAAASKPAVEIKQEGDTFYIKTSTTVRTTEINFKVGEEFEEQTVDGRPCKSLVKWESENKMVCEQKLLKGEGPKTSWTRELTNDGELILTMTADDVVCTRVYVREHHHHHH

SEQ ID NO: 2 - protein, 151 aa, synthetic construct
  REGION 1..151: note = Start-H6-TEV-CRABP2
MHHHHHHENLYFQGPNFSGNWKIIRSENFEELLKVLGVNVMLRKIAVAAASKPAVEIKQEGDTFYIKTSTTVRTTEINFKVGEEFEEQTVDGRPCKSLVKWESENKMVCEQKLLKGEGPKTSWTRELTNDGELILTMTADDVVCTRVYVRE

SEQ ID NO: 3 - protein, 336 aa, synthetic construct
  REGION 1..336: note = SnapTag-XTEN Linker-CRABP2-H6
DKDCEMKRTTLDSPLGKLELSGCEQGLHEIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLGSGSETPGTSESAPNFSGNWKIIRSENFEELLKVLGVNVMLRKIAVAAASKPAVEIKQEGDTFYIKTSTTVRTTEINFKVGEEFEEQTVDGRPCKSLVKWESENKMVCEQKLLKGEGPKTSWTRELTNDGELILTMTADDVVCTRVYVREHHHHHH

SEQ ID NO: 4 - protein, 380 aa, synthetic construct
  REGION 1..380: note = Start-SnapTag-XTEN Linker-CRABP2-H6
MDKDCEMKRTTLDSPLGKLELSGCEQGLHEIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLGSGSETPGTSESADKDCEMKRTTLDSPLGKLELSGCEQGLHEIIFLGKGTSAADAVEVPAPAAVLGGPEPLMQATAWLNAYFHQPEAIEEFPVPALHHPVFQQESFTRQVLWKLLKVVKFGEVISYSLAALAGNPAATAAVKTALSGNPVPILIPCHRVVQGDLDVGGYEGGLAVKEWLLAHEGHRLGKPGLGHHHHHH

SEQ ID NO: 5 - DNA, 38 bp, synthetic construct
  source 1..38: mol_type = other DNA; note = SnapTag
ttttggtctcacatggacaaagactgcgaaatgaaacg

SEQ ID NO: 6 - DNA, 58 bp, synthetic construct
  source 1..58: mol_type = other DNA; note = SnapTag
ttttggtctcatcagaggtacccggggtttcgctaccagaacccagacccggtttacc

SEQ ID NO: 7 - DNA, 36 bp, synthetic construct
  source 1..36: mol_type = other DNA; note = CRABP2
ttttggtctcacatgccgaactttagcggcaactgg

SEQ ID NO: 8 - DNA, 75 bp, synthetic construct
  source 1..75: mol_type = other DNA; note = CRABP2
ttttggtctcacatgcatcatcaccaccaccatgaaaacctatacttccaaggcccgaactttagcggcaactgg

SEQ ID NO: 9 - DNA, 43 bp, synthetic construct
  source 1..43: mol_type = other DNA; note = CRABP2
ttttggtctcactgaaagcgccccgaactttagcggcaactgg

SEQ ID NO: 10 - DNA, 38 bp, synthetic construct
  source 1..38: mol_type = other DNA; note = CRABP2
ttttggtctcacttattcacgaacatacacacgggtgc

SEQ ID NO: 11 - DNA, 56 bp, synthetic construct
  source 1..56: mol_type = other DNA; note = CRABP2
ttttggtctcacttaatggtggtggtgatgatgttcacgaacatacacacgggtgc

(19) United States
(12) Patent Application Publication
(10) Pub. No.: US 2025/0326723 A1
(43) Pub. Date: Oct. 23, 2025

(54) Development of an Enzyme-Inhibitor Reaction Using Cellular Retinoic Acid Binding Protein II for One-Pot Megamolecule Assembly

(71) Applicant: NORTHWESTERN UNIVERSITY, Evanston, IL (US)

(72) Inventors: Milan Mrksich, Hinsdale, IL (US); Blaise Russel Kimmel, Evanston, IL (US)

(21) Appl. No.: 18/703,425

(22) PCT Filed: Oct. 24, 2022
(86) PCT No.: PCT/US2022/078616

§ 371 Date: Apr. 22, 2024

Related U.S. Application Data
(60) Provisional application No. 63/270,922, filed on Oct. 22, 2021.

(51) Int. Cl. C07D 215/14; C07D 401/10; C07D 401/12; C07D 493/10

(52) U.S. Cl. CPC C07D 215/14; C07D 401/10; C07D 401/12; C07D 493/10

(57) ABSTRACT

The present disclosure relates generally to an enzyme building block for the assembly of megamolecules. The system is based on the inhibition of the human-derived cellular retinoic acid binding protein II (CRABP2) domain. The inventors synthesized a synthetic retinoid bearing an arylfluorosulfate group, which uses sulfur fluoride exchange click chemistry to covalently inhibit CRABP2. The inventors conjugated both the inhibitor and a fluorescein tag to an oligo(ethylene glycol) backbone and measured a second-order rate constant for the protein inhibition reaction of approximately 3,600 M⁻¹s⁻¹. The inventors used this new enzyme-inhibitor pair to assemble multi-protein structures in one-pot reactions using three orthogonal assembly chemistries to demonstrate exact control over the placement of protein domains within a single, homogeneous molecule. This work enables a new dimension of control over specificity, orientation, and stoichiometry of protein domains within atomically precise nanostructures.

### GOVERNMENT RIGHTS

This invention was made with U.S. government support under grant number DGE-1842165 awarded by the National Science Foundation and FA9550-16-1-0150 awarded by the Air Force Office of Scientific Research. The U.S. government has certain rights in the invention.

### SEQUENCE LISTINGS

This application contains, as a separate part of the disclosure, a Sequence Listing in computer-readable form (Filename: 2021-193P2_Seglisting.XML; Size: 14,578 bytes; Created: Sep. 20, 2022) which is incorporated by reference herein in its entirety.

### FIELD

The present disclosure relates generally to synthetic inhibitors, multi-functional linkers and megamolecule assemblies and methods of preparing and using same. More particularly, the present disclosure relates to a precisely-defined megamolecule assembly comprising at least one multi-functional linker having multiple branches, each branch having a synthetic inhibitor at its brain end, and multiple proteins or enzymes each protein or enzyme coupled to the synthetic inhibitor of one of the branches; methods of preparing the synthetic inhibitor and multi-functional linker; and methods of preparing the megamolecule assembly by one-pot reaction using the enzyme building block.

### INTRODUCTION

Megamolecules are large protein structures that have molecular weights approaching 1 MDa and dimensions of 100 nm and are perfectly defined in the placement of each atom and bond. These structures are assembled through reactions of linkers that are terminally substituted with covalent inhibitors and fusion proteins containing enzyme targets for the inhibitors. A benefit of this approach is that initial binding of the inhibitor to the enzyme brings the partners together in a rapid reaction and activates the inhibitor only when it is at the site of desired reaction. The inventors have used the reaction of a cutinase protein with a p-nitrophenyl phosphonate (pNPP) and a SnapTag domain with a chloro-pyrimidine (CP) inhibitor to synthesize dendritic molecules, therapeutic antibody mimics, and to study structure-function relationships in antibody-enzyme conjugates. The development of additional enzyme-inhibitor pairs will be important for enabling the efficient synthesis of complex protein architectures and possibly for preparing structures that are immunotolerant.

Accordingly, a need exists for new megamolecules that addresses one or more of the technical issues discussed above and new methods of making the same.

The present disclosure describes a method of preparing an irreversible inhibitor for cellular retinoic acid binding protein II (CRABP2) and a method of using the same in megamolecule assembly.

### SUMMARY

The present technology includes synthetic inhibitors, multi-functional linkers, and megamolecules and their methods of preparation and use.

One aspect of the disclosure provides a synthetic inhibitor comprising: a backbone comprising a unit (—R₁—) selected from the group of diphenylacetylene (Formula A), 4-(phenyl)ethynyl)-4,4-dimethyl-3,4-dihydroquinolin (Formula B), 4,4-dimethyl-6-(phenylethynyl)-1,2,3,4-tetrahydroquinoline (Formula C), phenyl acetylene (Formula D), a compound represented by Formula E, and a compound represented by Formula F as shown herein below; a first functional group (—R_{f1}) provided at a first end of the backbone; and a second functional group (—R_{f2}) provided at a second end of the backbone, the second functional group (—R_{f2}) comprising one of a carboxylic acid, —CO—CH₂CH₂—COOH, —CO—R₆—COOH, an amide, —CO—R₆—CO—NH—(PEG)ₘ-, an activated carboxylic acid, a n-hydroxysuccinimide (NHS) ester, an alcohol, a primary amine, a secondary amine, a fluorophore or fluorescein tag, a click chemistry molecule selected from the group of bicyclononyne (BCN), trans-cyclooctene (TCO), tetrazine (Tz), dibenzocyclooctyne (DBCO) and azide, and a combination thereof, wherein the —R₆— can be an aryl group or an alkyl having 1 to 6 carbon atoms, and m is in a range of 1-50, wherein the first functional group is specific for irreversibly inhibiting a bio-matter, and wherein each of R₂, R₃, R₄ and R₅ is independently selected from the group of an alkyl having 1 to 6 carbons (such as a methyl, ethyl, propyl, butyl, pentyl or hexyl group), a fluoro-alkyl having 1 to 6 carbons (such as a trifluoromethyl group, a difluoromethyl group, and a fluoromethyl group), and an aryl (such as a phenyl group), [symbol] represents a bond to the first functional group (—R_{f1}) or the second functional group (—R_{f2}), and n is in a range of 1 to 10, 1 to 5, or 1 to 3.

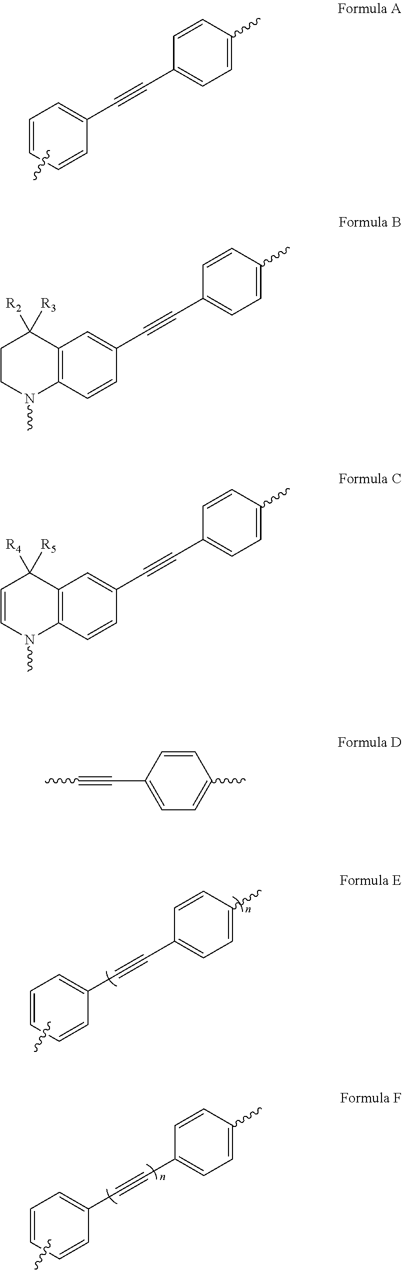

In embodiments, the first functional group (—R_{f1}) comprises a fluorosulfate (F—SO₂—O—), an arylfluorosulfate (F—SO₂—O—Ar), an arylsulfonyl fluoride (F—SO₂—Ar), a sulfonyl fluoride (F—SO₂—), ap-nitrophenyl phosphonate (pNPP), a chloro-pyrimidine (CP), a haloalkane, a benzylcytosine, a benzylguanine, an O⁶-benzylguanine, an α-haloalkane, a haloaromatic compound, a beta-lactam, an aglycone, a hydroxamic acid-benzophenone, a cognate oriT oligonucleotide sequence, a cysteine-reactive ATP-binding site inhibitor, a quinone methide, an α-halo phosphonic acid, a formylchromone, a cognate RNA sequence, adenosine, cytosine, a cognate DNA nicking site, a thiirane, a hydroxamic acid, an α-ketoxazole inhibitor, an electrophilic steroid, a phosphonate, a carbamate, an aromatic alkyne, beloranib, and a combination of the foregoing.

Another aspect of the disclosure provides a multi-functional linker comprising: a central compound having a core and at least two chain branches, each branch coupled to the core; and at least two inhibitors, each inhibitor coupled to one of the at least two chain branches, wherein the number of the at least two inhibitors and the number of the at least two chain branches are the same, and wherein each of the at least two inhibitors is specific for irreversibly inhibiting a specific bio-matter, such as a protein, an enzyme, an antibody fragment, a fusion protein, or a peptide, wherein each of the at least two inhibitors comprises a first functional group (—R_{f1}) specific for irreversibly inhibiting a specific bio-matter; and a second functional group (—R_{f2}), wherein each of the second functional groups (—R_{f2}) independently comprises one or more of a carboxylic acid, —CO—CH₂CH₂—COOH, —CO—R₆—COOH, an amide, —CO—R₆—CO—NH—(PEG)ₙ-, an activated carboxylic acid, a n-hydroxysuccinimide (NHS) ester, an alcohol, a primary amine, a secondary amine, a fluorophore or fluorescein tag, a click chemistry molecule selected from the group of bicyclononyne (BCN), trans-cyclooctene (TCO), tetrazine (Tz), dibenzocyclooctyne (DBCO) and azide, and a combination thereof, wherein the —R₆— can be an alkyl having up to 20 carbons or 1 to 6 carbons, or an aryl group, and n is in a range of 1 to 50.

In embodiments, each of the first functional groups (—R_{f1}) comprises a fluorosulfate (F—SO₂—O—), an arylfluorosulfate (F—SO₂—O—Ar), an arylsulfonyl fluoride (F—SO₂—Ar), a sulfonyl fluoride (F—SO₂—), a p-nitrophenyl phosphonate (pNPP), a chloro-pyrimidine (CP), a haloalkane, a benzylcytosine, a benzylguanine, an O⁶-benzylguanine, an α-haloalkane, a haloaromatic compound, a beta-lactam, an aglycone, a hydroxamic acid-benzophenone, a cognate oriT oligonucleotide sequence, a cysteine-reactive ATP-binding site inhibitor, a quinone methide, an α-halo phosphonic acid, a formylchromone, a cognate RNA sequence, adenosine, cytosine, a cognate DNA nicking site, a thiirane, a hydroxamic acid, an α-ketoxazole inhibitor, an electrophilic steroid, a phosphonate, a carbamate, an aromatic alkyne, beloranib, and a combination of the foregoing.

In embodiments, one of the at least two inhibitors is the synthetic inhibitor discussed above.

Another aspect of the disclosure provides a multi-functional linker comprising a central compound having a core, a first chain branch having a first end distal from the core, and a second chain branch having a second end distal to the core, each of the first and second chain branches coupled to the core; a first inhibitor comprising a first functional group specific for irreversibly inhibiting a first bio-matter, the first inhibitor coupled to the first end through a fourth functional group; and a second inhibitor comprising a second functional group specific for irreversibly inhibiting a second bio-matter, the second inhibitor coupled to the second end through a fifth functional group, wherein each of the fourth and fifth functional groups is independently a carboxylic acid, —CO—CH₂CH₂—COOH, —CO—R₆—COOH, an amide, —CO—R₆—CO—NH—(PEG)ₘ-, an activated carboxylic acid, a n-hydroxysuccinimide (NHS) ester, an alcohol, a primary amine, a secondary amine, a fluorophore or fluorescein tag, a click chemistry molecule selected from the group of bicyclononyne (BCN), trans-cyclooctene (TCO), tetrazine (Tz), dibenzocyclooctyne (DBCO) and azide, and a combination thereof, wherein the —R₆— can be an alkyl having up to 20 carbons or 1 to 6 carbons, or an aryl group, and m is in a range of 1 to 50. In embodiments, the first inhibitor is the synthetic inhibitor discussed herein above.

In embodiments, each of the first and second functional groups comprises a fluorosulfate (F—SO₂—O—), an arylfluorosulfate (F—SO₂—O—Ar), an arylsulfonyl fluoride (F—SO₂—Ar), a sulfonyl fluoride (F—SO₂—), ap-nitrophenyl phosphonate (pNPP), a chloro-pyrimidine (CP), a haloalkane, a benzylcytosine, a benzylguanine, an O⁶-benzylguanine, an α-haloalkane, a haloaromatic compound, a beta-lactam, an aglycone, a hydroxamic acid-benzophenone, a cognate oriT oligonucleotide sequence, a cysteine-reactive ATP-binding site inhibitor, a quinone methide, an α-halo phosphonic acid, a formylchromone, a cognate RNA sequence, adenosine, cytosine, a cognate DNA nicking site, a thiirane, a hydroxamic acid, an α-ketoxazole inhibitor, an electrophilic steroid, a phosphonate, a carbamate, an aromatic alkyne, beloranib, and a combination of the foregoing.

Another aspect of the disclosure provides a megamolecule comprising: a first bio-matter and a second bio-matter; and a multi-functional linker of the disclosure comprising: a central compound comprising a core, a first chain branch extending from the core and having a first end distal from the central compound, and a second chain branch extending from the core and having a second end distal from the central compound; a first inhibitor having a first functional group specific for irreversibly inhibiting the first bio-matter, and provided at the first end of the first chain branch; and a second inhibitor having a second functional group specific for irreversibly inhibiting the second bio-matter, and provided at the second end of the second chain branch, wherein the first bio-matter is coupled to the first inhibitor, the second bio-matter is coupled to the second inhibitor, and the megamolecule has a molecular weight in a range of about 1.0 KDa to about 10.0 MDa.

Another aspect of the disclosure provides a method of preparing a synthetic inhibitor having a structure of Formula 10 (4-(6-((4-((fluorosulfonyl)oxy)phenyl)ethynyl)-4,4-dimethyl-3,4-dihydroquinolin-1(2H)-yl)-4-oxobutanoic acid). The method includes admixing a compound having a structure of Formula 9 (4-(6-((4-hydroxyphenyl)ethynyl)-4,4-dimethyl-3,4-dihydroquinolin-1(2H)-yl)-4-oxobutanoic acid) with [4-(acetylamino)phenyl]imidodisulfuryl difluoride (AISF) to form the synthetic inhibitor having the structure of Formula 10. The method can further comprise preparing the compound having the structure of Formula 9 (4-(6-((4-hydroxyphenyl)ethynyl)-4,4-dimethyl-3,4-dihydroquinolin-1(2H)-yl)-4-oxobutanoic acid) by admixing a compound having a structure of Formula 4 (methyl-4(6-iodo-4,4-dimethyl-3,4-dihydroquinolin-1(2H)-yl)-4-oxobutanoate), Pd(PPh₃)₂Cl₂, CuI and a compound having a structure of Formula 7 (4-ethynylphenyl acetate) to form a compound having a structure of Formula 8 (methyl 4-(6-((4-(acetyloxy)phenyl)ethynyl)-4,4-dimethyl-3,4-dihydroquinolin-1(2H)-yl)-4-oxobutanoate), followed by saponification to form the compound having the structure of Formula 9. The method can further comprise preparing the compound having the structure of Formula 4 (methyl-4(6-iodo-4,4-dimethyl-3,4-dihydroquinolin-1(2H)-yl)-4-oxobutanoate).

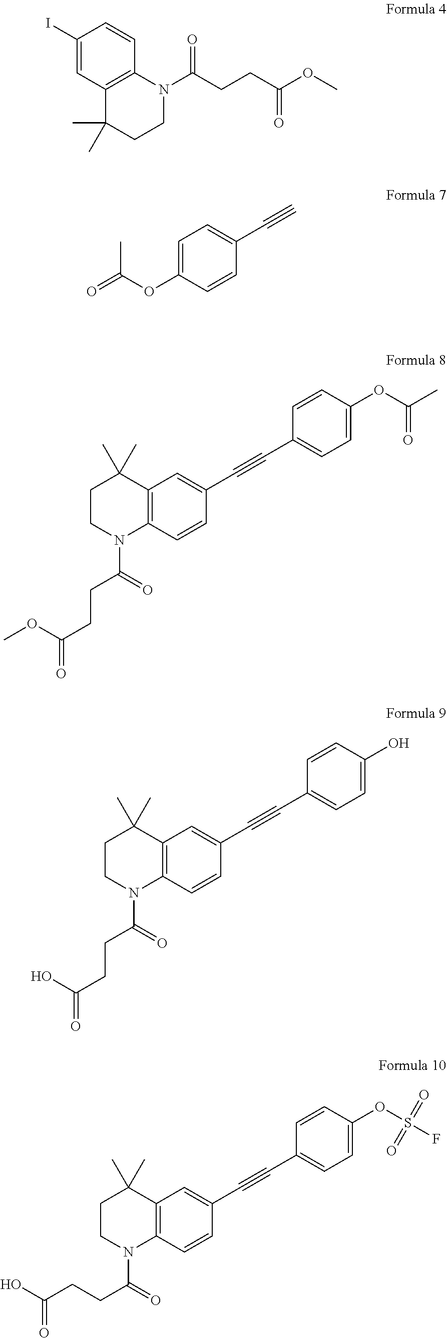

Another aspect of the disclosure provides a method of preparing a synthetic inhibitor having a structure of Formula 11 (Bi-EG(11)-(CrabTag Ligand, Fluorescein)), the method comprising: admixing the synthetic inhibitor having the structure of Formula 10 (4-(6-((4-((fluorosulfonyl)oxy)phenyl)ethynyl)-4,4-dimethyl-3,4-dihydroquinolin-1(2H)-yl)-4-oxobutanoic acid) and 6-[Fluorescein-5(6)-carboxamido]hexanoic acid in a container; adding an azido-PEG11-amine linker to the container; and reacting to form the synthetic inhibitor having the structure of Formula 11:

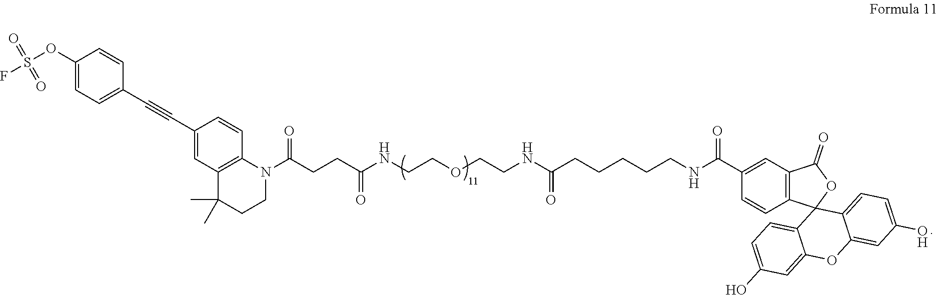

In embodiments, the admixing comprising dissolving the synthetic inhibitor having the structure of Formula 10 and 6-[Fluorescein-5(6)-carboxamido]hexanoic acid in a first solvent system in the first container; adding a second solvent to the container; and stirring for about 0.5-24 hours, or about 1-10 hours, or about 2-6 hours, or about 3-5 hours, or about 3.5-4.5 hours, or about 4 hours. In embodiments, the first solvent comprises 4-(4,6-dimethoxy-1,3,5-triazin-2-yl)-4-methyl-morpholinium chloride (DMTMM) and tetrahydrofuran (THF), and the second solvent comprises N-Methylmorpholine (NMM). In embodiments, the method further comprises removing DMTMM, THF and NMM from the container before adding the azido-PEG11-amine linker to the container. In embodiments, the reacting is conducted in a third solvent while stirring for about 6-24 hours, or about 8-20 hours, or about 12-16 hours, or about 16 hours to form the synthetic inhibitor having the structure of Formula 11. The third solvent is dimethylformamide (DMF) and NMM.

Another aspect of the disclosure provides a method for preparing a multi-functional linker of the disclosure, the method comprising: admixing a central compound having at least two chain branches with at least two inhibitors to form the multi-functional linker, wherein the first synthetic inhibitor couples to a first chain branch through a bond derived from a fourth functional group on the first synthetic inhibitor and a first end group on the first chain branch, and the second synthetic inhibitor couples to the second chain branch through a bond derived from a fifth functional group on the second synthetic inhibitor and a second end group on the second chain branch.

Another aspect of the disclosure provides a method for preparing a megamolecule of the disclosure, the method comprising: admixing a multi-functional linker of the disclosure with at least two bio-matters to form the megamolecule, wherein the megamolecule has a specific-defined structure and a molecule weight in a range of about 1.0 KDa to about 10.0 MDa. In embodiments, the megamolecule remains at least 95 wt. % intact after storage at room temperature in PBS solution at a concentration of 1 μM for up to 2 weeks.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1A illustrates a reaction for assembly of megamolecules. Covalent inhibition of cutinase with a p-nitrophenyl phosphonate (pNPP) inhibitor.

FIG. 1B illustrates a reaction for assembly of megamolecules. Covalent inhibition of SnapTag with a chloro-pyrimidine (CP) inhibitor.

FIG. 2 illustrates reactions for assembly of megamolecules. Covalent reaction of the CRABP2 (CrabTag) domain with a synthetic retinoid inhibitor according to the present disclosure.

FIG. 3 illustrates ESI-MS deconvoluted spectra with expected and observed molecular weights for CrabTag with a N-terminal Histag. Top: Purified CrabTag protein with the N-terminal histag and TEV protease recognition sequence (R, Black). Bottom: Adduct from reaction of R (0.1 nmol, 1 μM) with an inhibitor of the disclosure having a structure according to Formula 10 (5 eq., 5 μM) (Red). The reaction occurred in 100 μL of 1×PBS for 5 minutes before being stopped by addition of formic acid in preparation for ESI-MS.

FIG. 4 illustrates intact protein ESI-MS for CrabTag-inhibitor complexes. Reactions of C-terminal Histag R (1 μM, 1 equivalent) with either an inhibitor of the disclosure having a structure according to Formula 10 (5 μM, 5 equivalents) or an inhibitor of the disclosure having a structure according to Formula 11 (5 μM, 5 equivalents) occurred for 5 minutes before being stopped with formic acid.

FIG. 5 illustrates a representation of the crystal structure at 1.8 Å of a complex including an inhibitor of the disclosure having a structure according to Formula 10 and CrabTag (CRABP2-10). Tyr135 forms a covalent bond with the arylfluorosulfate electrophile within the inhibitor of the disclosure having a structure according to Formula 10, where the resulting Tyr˜Arg˜Arg motif is specified.

FIGS. 6A-6C illustrates 2Fo-Fc electron density maps for the inhibitor of the disclosure having a structure according to Formula 10 in the active site of CRABP2 at three different directions. Contoured at ±1.0 σ. Three directions shown (A-C) for the covalently bound inhibitor-tyrosine complex.

FIGS. 7A-7C illustrates Fo-Fc electron density maps for the crystal structure of the CRABP2-10 covalent complex at three different directions, contoured at ±1.0 σ. Three directions shown (A-C) for the covalently bound inhibitor-tyrosine complex.

FIGS. 8A-8D illustrates hydrogen bonding arrangements and distances within the Arg˜Arg˜Tyr binding motif at 1.8 Å resolution for the CRABP2-10 crystal structure. Four different orientations of the complex are shown.

FIGS. 9A-9D illustrates a crystal structure at 1.8 Å resolution for the CRABP2-10 complex detailing the Arg˜Arg˜Tyr motif with four orientations.

FIG. 10A illustrates kinetic characterization for the reaction of synthetic retinoid inhibitor 11, which is terminated in a fluorescein tag (green).

FIG. 10B illustrates the reaction of CrabTag with linker 11 at different time points during the reaction. Reaction products were separated using SDS-PAGE and the fluorescent product band was quantitated using ImageJ.

FIG. 10C illustrates deconvoluted protein mass spectrometry data quantifying the molecular weight of CrabTag (R, top) before and after complexation with either inhibitor 10 (middle) or inhibitor 11 (bottom).

FIG. 10D illustrates the fraction of covalently occupied CrabTag protein for each linker concentration at discrete time points to give the observed reaction rate (kobs). All plots and values have error represented as SEM.

FIG. 10E illustrates the kobs data, plotted relative to the linker concentrations in a Michaelis-Menten analysis and constants ki, KI, keff. All plots and values have error represented as SEM.

FIG. 11 illustrates time-lapse stability for a complex including an inhibitor of the disclosure having a structure according to Formula 11 and CrabTag (CrabTag-11) at room temperature over a period of two weeks. Normalized covalent occupancy was greater than 95% at the end of the two-week period. Control group is the CrabTag protein domain without an inhibitor of the disclosure having a structure according to Formula 11 (fluorescent linker 11).

FIG. 12A illustrates one-pot megamolecule assembly from building blocks into a compound having a structure of Configuration 16.

FIG. 12B Top: Brightfield SDS-PAGE of the reaction of FIG. 12A over a two-hour time scale. Benchmark protein ladder for relative scale (Bottom to top: represents Cutinase; the compounds having Formulas 12; 13; 14; 15; and 16 respectively). Bottom: Fluorescent image of SDS-PAGE (Bottom to top: represents the compounds having Formulas 12; 13; 14; 15; and 16 respectively).

FIG. 12C is a plot showing the relative abundance of all reaction intermediates over time as quantified using both brightfield and fluorescent SDS-PAGE.

FIG. 13A illustrates a block diagram for the proposed reaction network to prepare the compound having the structure of Configuration 16 as shown in FIG. 12A.

FIG. 13B illustrates concentration profiles for the observed and simulated fit data for the one-pot reaction to prepare the compound having the structure of Configuration 16 as shown in FIG. 12A with estimated kinetic rate constants. Error is plotted and represented in the kinetic rate constants as SEM.

FIG. 14 illustrates model and simulation residual values plotted against time for all species. In this figure, 1A is compound 12 from the examples, 1B is compound 13 from the examples, 2A is compound 14 from the examples, 2B is compound 15 from the examples, and 3 is compound 16 from the examples.

FIG. 15 illustrates model and simulation observed values plotted against predicted values for all species. In this figure, 1A is compound 12 from the examples, 1B is compound 13 from the examples, 2A is compound 14 from the examples, 2B is compound 15 from the examples, and 3 is compound 16 from the examples.

FIG. 16 illustrates one-pot megamolecule reaction to prepare the compound having the structure of Configuration 18 of the examples using heterotrifunctional linker having the structure of Formula 17 from the examples. The core (grey portion) of the protein scaffold is trifunctional linker 17 from the examples, which bears each of the three covalent inhibitors. The red portion represents the CRABP2 (CrabTag), the blue portion represents cutinase, and the yellow portion represents SnapTag

FIG. 16B is the SDS-PAGE characterization of the enzyme reactants and purified one-pot megamolecule product having the structure of Configuration 18.

FIG. 16C is the ESI-MS characterization of the enzyme reactants and purified one-pot megamolecule product having the structure of Configuration 18.

FIG. 16D is the one-pot reaction synthesis of double-branched megamolecule 19 of the examples using a di-SnapTag fusion protein as the scaffold core.

FIG. 16E is the SDS-PAGE characterization of the starting proteins and one-pot megamolecule product having the structure of Configuration 19.

FIG. 16F is the analysis of partition coefficients (Kav) based on size-exclusion chromatograms from protein and megamolecule purification.

FIG. 17 illustrates ESI-MS of the purified SnapTag-SnapTag (SS) fusion protein.

FIG. 18A illustrates orthogonality of enzyme-inhibitor pairs in the megamolecule assembly toolbox. A Coomassie stain with brightfield view of SDS-PAGE. Lane 1: CRABP2 (R) protein domain (MW, −16 kDa). Lane 2: Reaction of CRABP2 with fluorescent linker 11. Lane 3: Cutinase-SnapTag (CS) fusion protein (MW, −45 kDa). Lane 4: CS with the CrabTag fluorescent inhibitor. Lane 5: CS with the di-cutinase PEG11 linker to yield a di-CS megamolecule (MW, −90 kDa). Lane 6: CS with the di-SnapTag PEG11 linker to yield a di-CS megamolecule (MW, −90 kDa). Lanes 7-8: CS was first mixed with the CrabTag fluorescent inhibitor for two hours. Afterwards, the di-cutinase PEG11 or the di-SnapTag PEG11 linker was added, forming the di-CS megamolecule (MW, −90 kDa). The next day, the R protein was added to form the fluorescent band shown in the fluorescent SDS-PAGE. Lanes 9-10: R was first mixed with either the di-cutinase PEG11 or the di-SnapTag PEG11 linker for two hours, followed by the addition of the CS fusion protein to form the di-CS megamolecule (MW, −90 kDa). The following day, the fluorescent CrabTag linker was added to form the fluorescent band shown in the fluorescent SDS-PAGE. Lane 11: Benchmark protein ladder. For all reactions, an excess of protein was used.

FIG. 18B is a fluorescent SDS-PAGE view. Lane 1: CRABP2 (R) protein domain (MW, −16 kDa). Lane 2: Reaction of CRABP2 with fluorescent linker 11 to yield the fluorescent band seen in the bottom gel. Lane 3: Cutinase-SnapTag (CS) fusion protein (MW, −45 kDa). Lane 4: CS with the CrabTag fluorescent inhibitor. Lane 5: CS with the di-cutinase PEG11 linker to yield a di-CS megamolecule (MW, −90 kDa). Lane 6: CS with the di-SnapTag PEG11 linker to yield a di-CS megamolecule (MW, −90 kDa). Lanes 7-8: CS was first mixed with the CrabTag fluorescent inhibitor for two hours. Afterwards, the di-cutinase PEG11 or the di-SnapTag PEG11 linker was added, forming the di-CS megamolecule (MW, −90 kDa). The next day, the R protein was added to form the fluorescent band shown in the fluorescent SDS-PAGE. Lanes 9-10: R was first mixed with either the di-cutinase PEG11 or the di-SnapTag PEG11 linker for two hours, followed by the addition of the CS fusion protein to form the di-CS megamolecule (MW, −90 kDa). The following day, the fluorescent CrabTag linker was added to form the fluorescent band shown in the fluorescent SDS-PAGE. Lane 11: Benchmark protein ladder. For all reactions, an excess of protein was used.

FIG. 19 illustrates context-specific reactivity of the CRABP2 protein domain (MW, −16 kDa) (Lane A) with the di-cutinase PEG11 (Lane B) and di-SnapTag PEG1l linkers (Lane C). Protein benchmark ladder on left for reference.

FIG. 20 illustrates different types of megamolecules, represented as Configuration 16, Configuration 18, Configuration 19, Configuration 30, Configuration 31, Configuration 32, and Configuration 33. The central grey portion represents the multi-functional linker, the red portion represents the CRABP2 (CrabTag), the blue portion represents cutinase, the yellow portion represents SnapTag, and the green portion represents a fluorescein tag.

### DETAILED DESCRIPTION

The following description of technology is merely exemplary in nature of the subject matter, manufacture, and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description.

The present disclosure provides synthetic inhibitors, multi-functional linkers and megamolecules assemblies and their methods of preparation and use. Advantageously, the methods disclosed herein enable a new dimension of control over specificity, orientation, and stoichiometry of protein domains within atomically precise nanostructures.

As used herein, the term “megamolecule” refers to a large protein structures that have molecular weights approaching 1 MDa and dimensions of 100 nm. In general, megamolecules and are precisely defined in terms of the placement of each atom and bond. These structures can be assembled through reactions of linkers that are terminally substituted with inhibitors specific to enzymes and fusion proteins containing enzyme targets specific to the inhibitors. In this way, the precise structure of the megamolecule can be controlled and prepared with specificity.

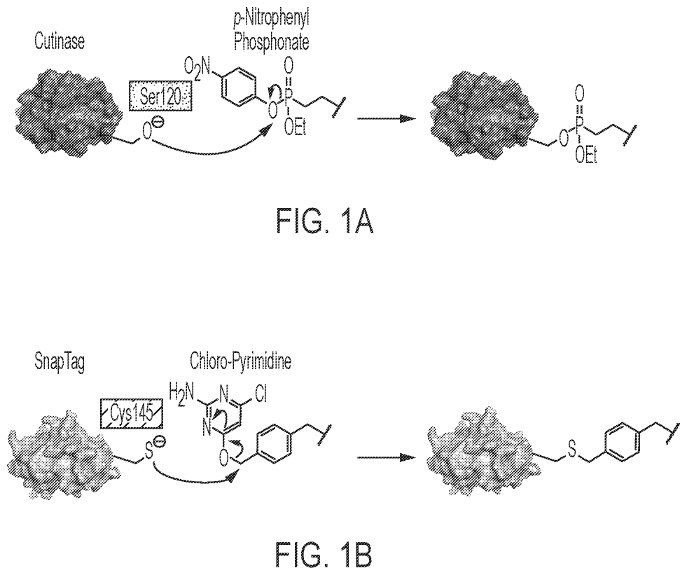

Applicant has developed methods of reacting a cutinase protein with a p-nitrophenyl phosphonate (pNPP) and reacting a SnapTag domain with a chloro-pyrimidine (CP) inhibitor to synthesize dendritic molecules, therapeutic antibody mimics, and to study structure-function relationships in antibody-enzyme conjugates as shown in FIGS. 1A and 1B. The development of additional enzyme-inhibitor pairs will be important for enabling the efficient synthesis of complex protein architectures and possibly for preparing structures that are immunotolerant. The present disclosure describes the development of an irreversible inhibitor for cellular retinoic acid binding protein II (CRABP2) and demonstrate its use in megamolecule assembly.

### Synthetic Inhibitors

The disclosure generally provide a synthetic inhibitor, such as a synthetic retinoid. The synthetic inhibitor can include a highly conjugated linear backbone (—R₁—); a first functional group (—R_{f1}) provided at a first end of the highly conjugated linear backbone; and a second functional group (—R_{f2}) provided at a second end of the highly conjugated linear backbone, such that the synthetic inhibitor can be represented by the general formula R_{f1}—R₁—R_{f2}. As used herein, the term “conjugated backbone” refers to a backbone of alternating single and multiple bonds result in π-conjugation by overlap of the π-orbitals, giving rise to a continuum of energy states. As used herein, the term “highly conjugated linear backbone” refers to a linear backbone of alternating single and multiple bonds resulting in π-conjugation by overlap of the π-orbitals, giving rise to a continuum of energy states, and the conjugation of the backbone results in a rigid linear structure, wherein the at least about 50% of the carbon atoms in the backbone are part of the conjugates structure. In various embodiments, at least about 60%, at least about 70%, at least about 80%, at least about 90%, at least about 95%, about 90%-100%, or 100% of the carbon atoms on the backbone of a highly conjugated linear backbone are conjugated. Non-limiting examples of “highly conjugated linear backbone” are backbones having structures according to Formula A to Formula F described herein.

In embodiments, the highly conjugated linear backbone maybe or comprise a unit (—R₁—) selected from the group of diphenylacetylene (Formula A), 4-(phenyl)ethynyl)-4,4-dimethyl-3,4-dihydroquinolin (Formula B), 4,4-dimethyl-6-(phenylethynyl)-1,2,3,4-tetrahydroquinoline (Formula C), phenyl acetylene (Formula D), Formula E, and Formula F, as shown below, wherein, each of R₂, R₃, R₄ and R₅ is independently selected from the group of an alkyl group with 1 to 6 carbons, a fluoro-alkyl group having 1 to 6 carbons, and an aryl group, wherein n is in a range of 1 to 10 and [symbol] represents a bond to the first functional group (—R_{f1}) or the second functional group (—R_{f2}):

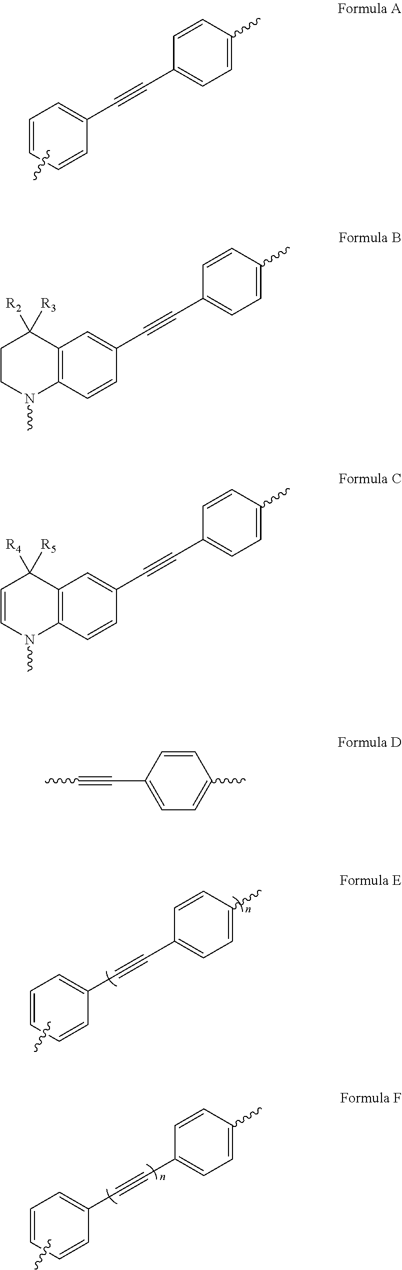

In embodiments, each of R₂, R₃, R₄ and R₅ is independently selected from the group of methyl, ethyl, propyl, isopropyl, butyl, tert-butyl, iso-butyl, pentyl, iso-pentyl, 1,1-dimethylpropyl, 2,2-dimethylpropyl, 1,2-dimethylpropyl, cyclopentyl, hexyl, 1,1-dimethylbutyl, 2,2-dimethylbutyl, 3,3-dimethylbutyl, 1,2-dimethylbutyl, 1,3-dimethylbutyl, 2,3-dimethylbutyl, 1,1,2-trimethylpropyl, 1,2,2-trimethylpropyl, 2-methylcyclopentyl, 3-methylcyclopentyl, 4-methylcyclopentyl, cyclohexyl. In embodiments, each of R₂, R₃, R₄ and R₅ is independently selected from the group of methyl, ethyl, propyl, butyl, pentyl, and hexyl. In embodiments, each of R₂, R₃, R₄ and R₅ is independently selected from substituted and unsubstituted phenyl. In embodiments, R₂ is an alkyl group with 1 to 6 carbons and R₃ is phenyl. In embodiments, R₂ is a fluoro-alkyl group with 1 to 6 carbons and R₃ is phenyl. In embodiments, R₂ is an alkyl group with 1 to 6 carbons and R₃ is a fluoro-alkyl group with 1 to 6 carbons. In embodiments, each of R₂ and R₃ is an alkyl group with 1 to 6 carbons. In embodiments, R₂ and R₃ are each methyl. In embodiments, R₂ and R₃ are each ethyl. In embodiments, R₄ is an alkyl group with 1 to 6 carbons and R₅ is phenyl. In embodiments, R₄ is a fluoro-alkyl group with 1 to 6 carbons and R₅ is phenyl. In embodiments, R₄ is an alkyl group with 1 to 6 carbons and R₅ is a fluoro-alkyl group with 1 to 6 carbons. In embodiments, each of R₄ and R₅ is an alkyl group with 1 to 6 carbons. In embodiments, R₄ and R₅ are each methyl. In embodiments, R₄ and R₅ are each ethyl. In embodiments, n is 1 to 5. In embodiments, n is 1 to 3. In embodiments, n is 1.

In embodiments, the highly conjugated linear backbone may be or comprise one of diphenylacetylene (Formula A), 4-(phenyl)ethynyl)-4,4-dimethyl-3,4-dihydroqiinolin (Formula B), and 4,4-dimethyl-6-(phenylethynyl)-1,2,3,4-tetrahydroquinoline (Formula C), wherein, each of R₂, R₃, R₄ and R₅ is independently selected from the group of an alkyl group with 1 to 6 carbons, a fluoro-alkyl group having 1 to 6 carbons, and an aryl group, and [symbol] represents a bond to the first functional group (—R_{f1}) or the second functional group.

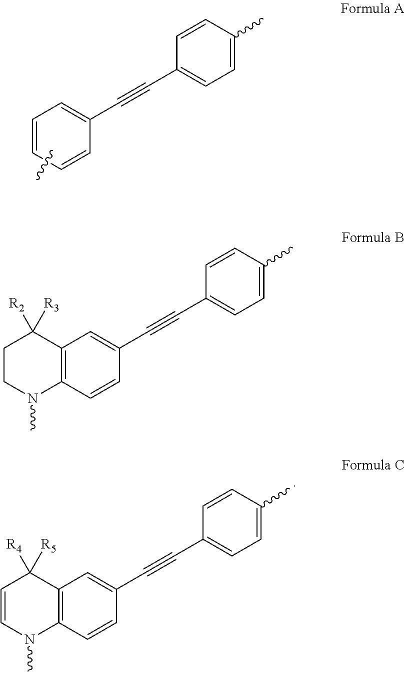

In refinements of the foregoing embodiment, R₂ is an alkyl group with 1 to 6 carbons and R₃ is phenyl. In refinements of the foregoing embodiment, R₂ is a fluoro-alkyl group with 1 to 6 carbons and R₃ is phenyl. In refinements of the foregoing embodiment, R₂ is an alkyl group with 1 to 6 carbons and R₃ is a fluoro-alkyl group with 1 to 6 carbons. In refinements of the foregoing embodiment, each of R₂ and R₃ is an alkyl group with 1 to 6 carbons. In refinements of the foregoing embodiment s, R₂ and R₃ are each methyl. In refinements of the foregoing embodiment, R₂ and R₃ are each ethyl. In refinements of the foregoing embodiment, R₄ is an alkyl group with 1 to 6 carbons and R₅ is phenyl. In refinements of the foregoing embodiment, R₄ is a fluoro-alkyl group with 1 to 6 carbons and R₅ is phenyl. In refinements of the foregoing embodiment, R₄ is an alkyl group with 1 to 6 carbons and R₅ is a fluoro-alkyl group with 1 to 6 carbons. In refinements of the foregoing embodiment, each of R₄ and R₅ is an alkyl group with 1 to 6 carbons. In refinements of the foregoing embodiment, R₄ and R₅ are each methyl. In refinements of the foregoing embodiment, R₄ and R₅ are each ethyl.

In embodiments, the synthetic inhibitor comprises an inhibitor having a structure according to Formula G, Formula H, or Formula I.

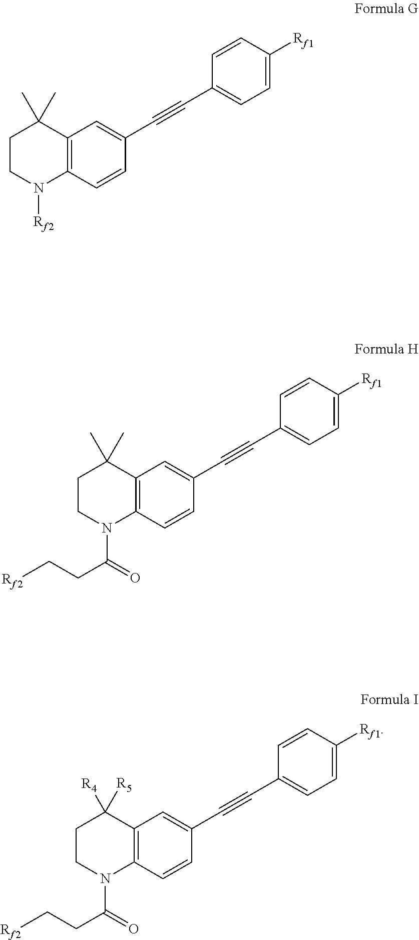

The first functional group (—R_{f1}) is provided at the first end of the highly conjugated linear backbone and can be any functional group specific for reversibly or irreversibly inhibiting an enzyme, protein, antibody fragment, fusion protein, or peptide. In embodiments, the first functional group is specific for irreversibly inhibiting or coupling to an enzyme, protein, antibody fragment, fusion protein, or peptide. As used herein and unless specified otherwise, the terms “coupled,” “couple,” or “coupling” encompass any one or more of covalent bond formation, hydrogen bond formation, ionic bond formation (e.g., electrostatic attraction), and van der Waals interactions for example, through which a protein or enzyme can reversibly or irreversibly associate with the functional group at the linker terminus. As used herein, the term “enzyme” refers to a protein that acts as biological catalyst; the term “enzyme” encompasses traditional enzymes and single-turnover enzymes commonly referred to as “suicide enzymes.” As used herein, the term “irreversible inhibitor” for an enzyme or a protein refers to a substance permanently inactivates the enzyme, usually through a covalent bond, such that no other enzyme-substrate complex can form.

The inventors previously described fusion proteins and fusion protein constructs in U.S. Patent Publication No. 2019/0161556, which is hereby incorporated by reference in its entirety for all purposes.

In embodiments, the first functional group comprises a fluorosulfate group (F—SO₂—O—), an arylfluorosulfate group (F—SO₂—O—Ar), an arylsulfonyl fluoride group (F—SO₂—Ar), or a sulfonyl fluoride group (F—SO₂—). In embodiments, the first functional group comprises an aryl fluorosulfate group (F—SO₂—O—Ar) or an arylsulfonyl fluoride group (F—SO₂—Ar). In embodiments, the first functional group comprises a fluorosulfate group (F—SO₂—O—) or an aryl fluorosulfate group (F—SO₂—O—Ar). In embodiments, the first functional group comprises an aryl fluorosulfate group (F—SO₂—O—Ar). Thus, in embodiments, the synthetic inhibitor comprises a fluorosulfate electrophile and has a chemical formula (F—SO₂—O—R₁—R₂). In embodiments, the first functional group comprises a fluorosulfate or an arylfluorosulfate which enables sulfur fluoride exchange click chemistry to specifically, selectively, and irreversibly inhibit cellular retinoic acid binding protein II (CRABP2) (CrabTag).

In embodiments, the first functional group (—R_{f1}—) can be selected from the group of p-nitrophenyl phosphonate, a chloro-pyrimidine (CP), a haloalkane, a benzylcytosine, a benzylguanine, O⁶-benzylguanine, α-haloalkane or derivative thereof, haloaromatic compound or derivative thereof, beta-lactam or derivative thereof such as clavulanic acid or derivative thereof, aglycone or derivative thereof, hydroxamic acid-benzophenones or derivative thereof, cognate oriT oligonucleotide sequence, cysteine-reactive ATP-binding site inhibitors, quinone methides or derivative thereof, α-halo phosphonic acids or precursor or derivative thereof, formylchromones or derivative thereof, cognate RNA sequence, adenosine or derivative thereof, cytosine or derivative thereof, cognate DNA nicking sites, thiiranes or derivative thereof, hydroxamic acids or derivative thereof, α-ketoxazole inhibitor or derivative thereof, electrophilic steroid, phosphonates, carbamates, aromatic alkynes, beloranib or derivative thereof, and combinations of the foregoing.

In embodiments, the first functional group is selected from the group of p-nitrophenyl phosphonate, O⁶-benzylguanine, α-haloalkane or derivative thereof, haloaromatic compound or derivative thereof, beta-lactam or derivative thereof such as clavulanic acid or derivative thereof, aglycone or derivative thereof, hydroxamic acid-benzophenones or derivative thereof, cognate oriT oligonucleotide sequence, and combinations thereof. In embodiments, the first functional group is selected from the group of a fluorosulfate, an arylfluorosulfate, p-nitrophenyl phosphonate, O⁶-benzylguanine, α-haloalkane or derivative thereof, haloaromatic compound or derivative thereof, and combinations thereof. In embodiments, the first functional group can be selected from a fluorosulfate, an arylfluorosulfate, p-nitrophenyl phosphonate, a cognate oriT oligonucleotide sequence, or a combination thereof.

In general, the second functional group (—R_{f2}) can be any group capable of reacting with and coupling to an end group of a chain branch of a multi-functional linker disclosed herein and/or any group suitable for imagining or diagnostics. For example, the second functional group can be a fluorescent group such that the synthetic inhibitor (and any protein or enzyme attached thereto) can be detected by fluorescence spectroscopy. Suitable second functional groups for coupling with an end group of a chain branch of a multi-functional linker disclosed herein can include, but are not limited to, carboxylic acids or esters thereof, alcohols, amides, primary amines, secondary amines, N-hydroxysuccinimide (NHS) or esters thereof, and click chemistry functional groups such as alkynes, tetrazines, alkenes, azides, nitrones, and tetrazoles. In embodiments, the second functional group (—R_{f2}) comprises a carboxylic acid, an amide, an alcohol, an alkyne, a tetrazine, an azide, or an ester of NHS. In embodiments, the carboxylic acid comprises 4-oxobutanoic acid (—CO—CH₂CH₂—COOH) or a group having a structure according to the formula —CO—R₆—COOH. In embodiments, the carboxylic acid comprises an activated carboxylic acid. In embodiments, the amide comprises a group having a structure according to the formula —CO—R₆—CO—NH—(PEG)ₙ-. In embodiments, the second functional group (—R_{f2}) comprises a click chemistry molecule group including a moiety selected from a bicycle[6.1.0]nonyne (BCN), a trans-cycloctene (TCO), tetrazine, diarylcyclooctyne (DBCO) and azide. In embodiments, the second functional group (—R_{f2}) comprises a fluorophore or fluorescein tag.

In any of the foregoing embodiments, the —R₆— can be an alkyl having up to 20 carbon atoms, for example or 1 to 6 carbon atoms, or an aryl group. In embodiments, n can be in a range of 1 to 50, 1 to 40, 1 to 32, 1 to 30, or 1 to 23, such as 1 (a single ethylene glycol (EG) group), 3, 4, 6, 7, 9, 10, 11, 12, 23 or 32. The -(PEG)ₙ- may have a molecular weight up to 20K, such as EG 456 (20 k). Non-limiting examples of the amide group having a structure according to the formula —CO—R₆—CO—NH—(PEG)ₙ- include any of —CO—R₆—CO—NH—(PEG)ₙ-BDCO, —CO—R₆—CO—NH—(PEG)ₙ-azide, and —CO—R₆—CO—NH—(PEG)ₙ-TCO.

In embodiments, the second functional group comprises one of carboxylic acid, 4-oxobutanoic acid (—CO—CH₂CH₂—COOH), a group having the structure according to formula —CO—R₆—COOH, and a group having the structure according to formula —CO—R₆—CO—NH—(PEG)ₙ-.

The synthetic inhibitor can generally be any structure having the formula R_{f1}—R₁—R_{f2}, as defined herein. In embodiments, the synthetic inhibitor can be a synthetic retinoid, such as, but not limited to 4-(6-((4-((fluorosulfonyl)oxy)phenyl)ethynyl)-4,4-dimethyl-3,4-dihydroquinolin-1(2H)-yl)-4-oxobutanoic acid having a chemical structure according to Formula 10, a compound having a structure according to Formula 11 (Bi-EG(11)-(CrabTag Ligand, Fluorescein)), or an inhibitor having a structure according to any of Formula J, Formula K, Formula L, Formula M, Formula N, and Formula 0, wherein n is in a range of 1 to 100. In embodiments, n can be in a range of 1-100, 1-50, 1-40, 1-32, or 1-23. For example, n can be in a range of 1 to 40, such as 1 (a single ethylene glycol (EG) group), 3, 4, 6, 7, 9, 10, 11, 12, 23 or 32. The -(PEG)ₙ- may have a molecular weight up to 20K, such as EG 456 (20 k).

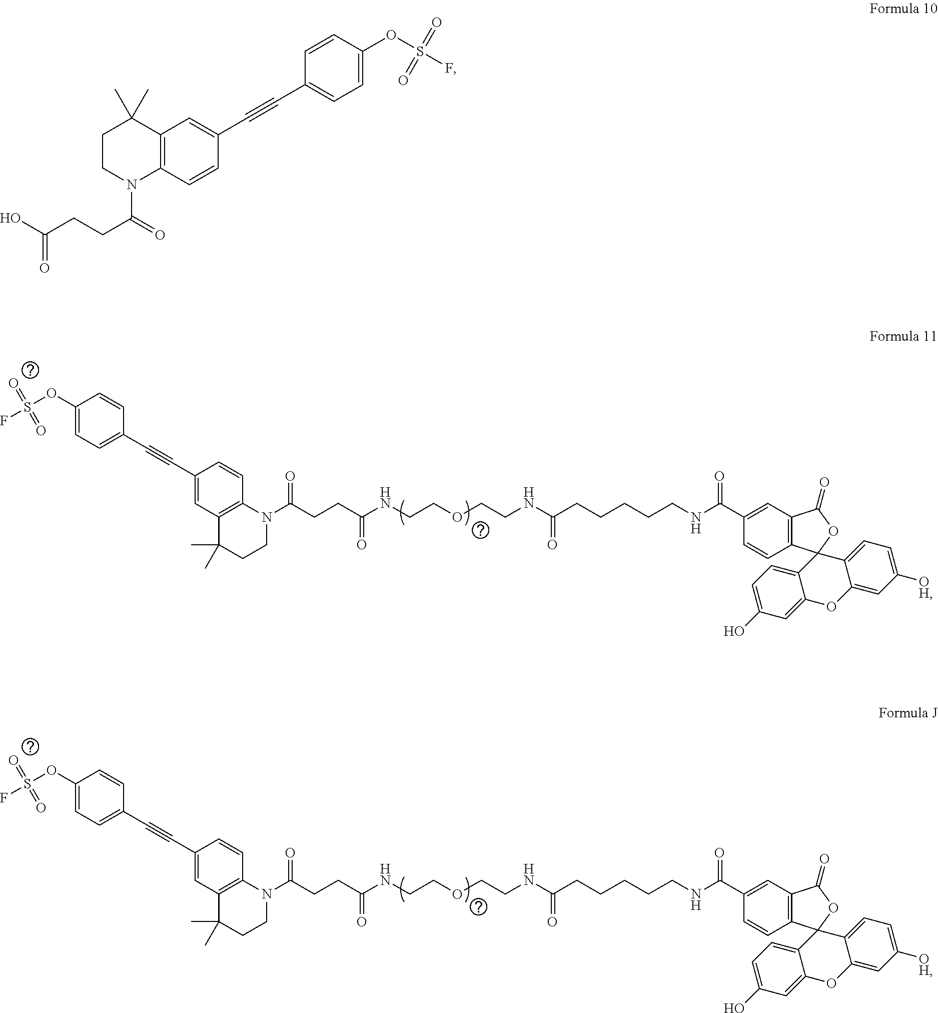

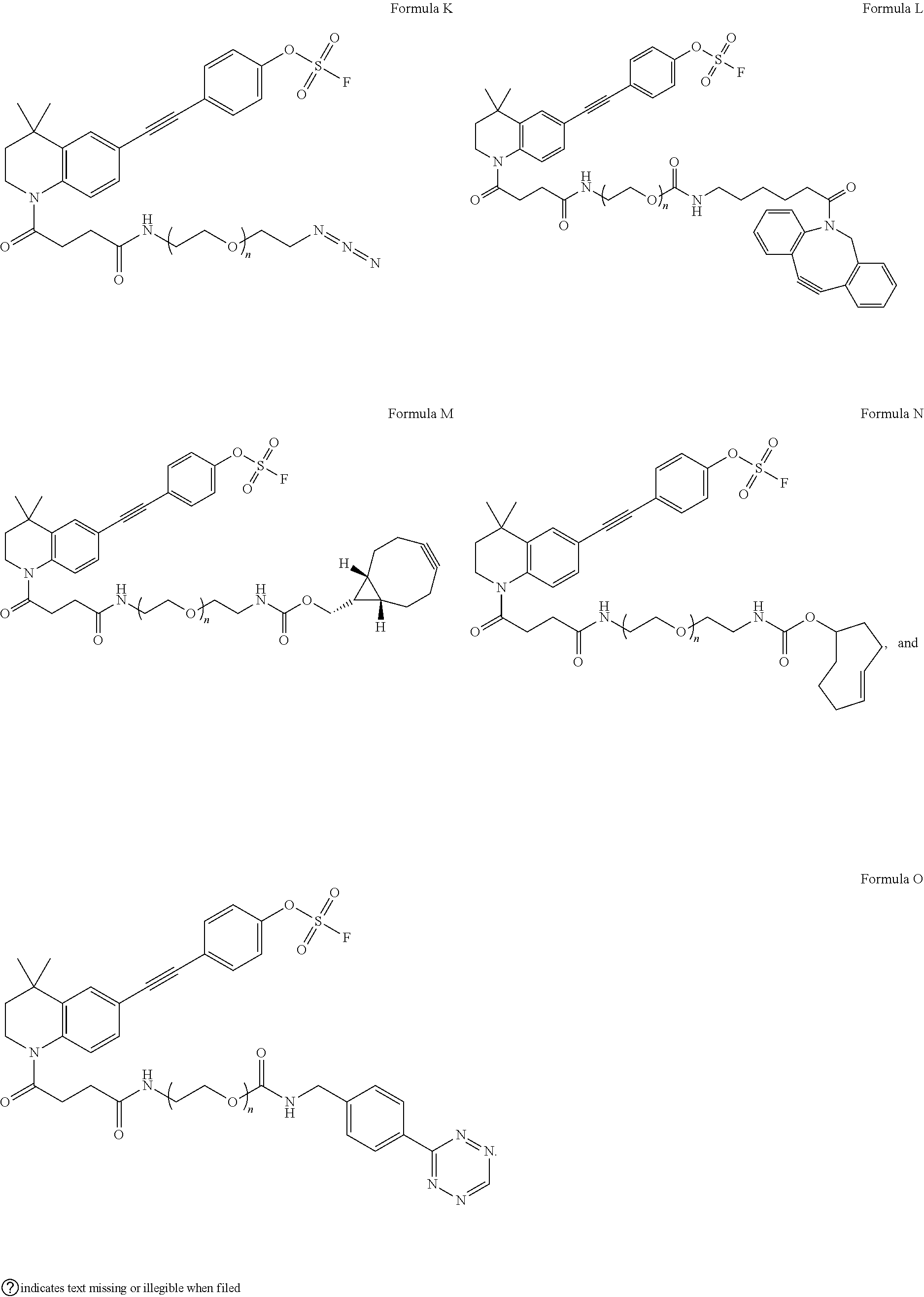

In embodiments, the synthetic inhibitor is the compound having the structure of Formula 10 (4-(6-((4-((fluorosulfonyl)oxy)phenyl)ethynyl)-4,4-dimethyl-3,4-dihydroquinolin-1(2H)-yl)-4-oxobutanoic acid).

In embodiments, the synthetic inhibitor is one of the synthetic inhibitors having the structures of Formula 11 and Formula J-Formula O each of which is a fluorescent linker having a fluorophore or fluorescent tag which can enable the fluorescent linker for imaging and diagnostic applications and purposes. These fluorescent linker can also be used for characterizing and/or quantifying the reaction kinetics of protein inhibition reaction by the linker by measurement of the changes of the fluorescence intensity during the reaction over the reaction time.

### Multifunctional Linker

The present disclosure further provides a multi-functional linker. The multi-functional linker generally includes a central compound having at least two chain branches, each chain branch including an inhibitor. In embodiment, at least one of the inhibitor is the synthetic inhibitor of the disclosure.

In general, the central compound includes a core and at least two chain branches. In embodiment, the central compound can be a star-shaped compound. The core can generally be any compound to which two or more branches are attached and provides a central axis for the branches. Typically, the core includes an aromatic or non-aromatic cyclic structure. Non-limiting examples of core compounds include differently membered aromatic rings or non-aromatic rings (such as aliphatic rings) bearing two or more carboxyl groups, primary amine groups or secondary amine groups. Non-limiting examples of the aliphatic rings include cycloalkane having 3-30 carbon atoms, such as cyclohexane and cyclooctane. Non-limiting examples of the aromatic rings include benzene, 1,3,5-triphenyl benzene, naphthalene, phenanthrene, anthracene, phenanthrene, pyrene, chrysene, beno[a]pyrene, fluoranthene, bi-phenyl or a compound having 2 or more phenyl or naphthalene groups. In embodiments, the central compound comprises a 1,3,5-benzenetricarboxylate core (Core 1); a compound having a structure of Core 2 including 1,3,5-tri(4-carboxyphenyl)benzene (Core 3) or 1,3,5-tri(4-aminophenyl)benzene (Core 4); a compound having a structure of Core 5, 1,3,5-tri-phenylphosphin having a structure of (Core 6); a compound having a structure of Core 7, Core 8, Core 9, Core 10, or Core 11, as shown below, wherein each of R, R₁, R₂, R₃, R₄, R₅, and R₆ can independently be carboxyl group, primary amine group or secondary amine group. In embodiments, R₁, R₂, R₃, R₄, R₅, and R₆ in one core compound can be the same.

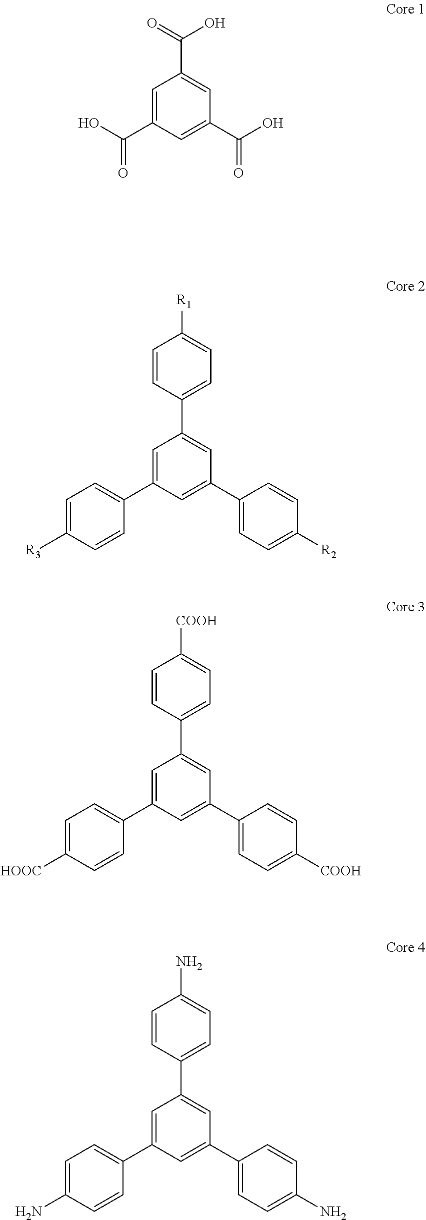

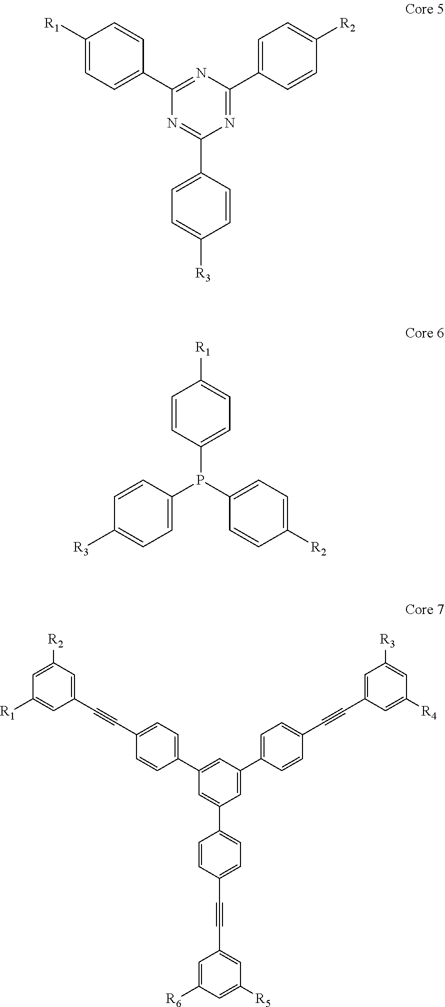

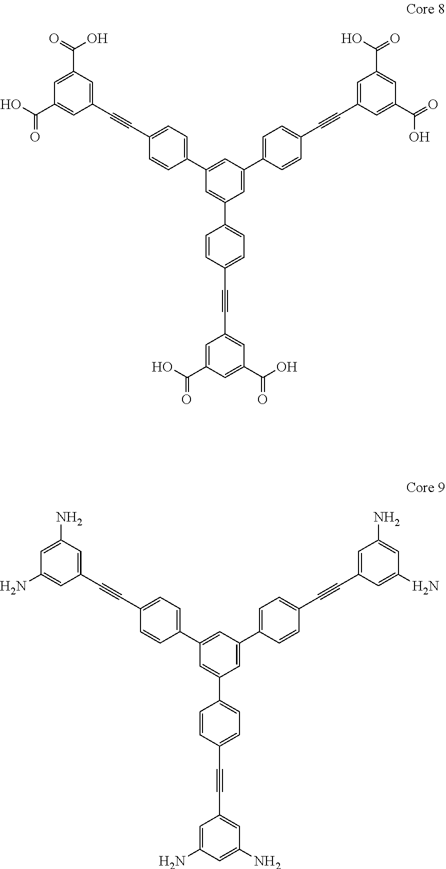 [SMILES: O=C(O)c1cc(C#Cc2ccc(-c3cc(-c4ccc(C#Cc5cc(C(=O)O)cc(C(=O)O)c5)cc4)cc(-c4ccc(C#Cc5cc(C(=O)O)cc(C(=O)O)c5)cc4)c3)cc2)cc(C(=O)O)c1 ; Nc1cc(N)cc(C#Cc2ccc(-c3cc(-c4ccc(C#Cc5cc(N)cc(N)c5)cc4)cc(-c4ccc(C#Cc5cc(N)cc(N)c5)cc4)c3)cc2)c1]

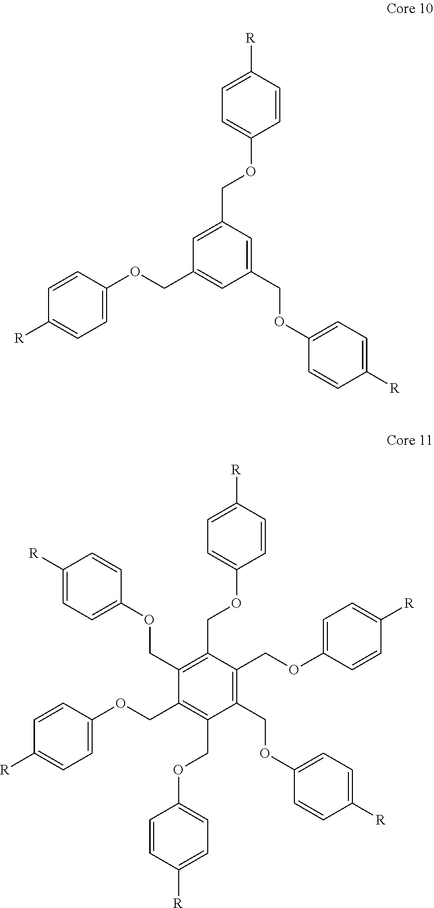

The central compound further includes at least two chain branches. Each branch has one end that is proximal to and attached to the core compound and a second end that is distal to the core compound. The chain branches can be arranged in a radial pattern around a central node (core compound). In embodiments, the multi-functional linker comprises at least 2 chain branches, at least 3 chain branches, or 2-20, 2-8, 2-6, 3-6, 3-5, or 3 chain branches. In general, the length of each chain branch can be any length suitable to allow a synthetic inhibitor to be coupled to the end of each chain branch. Without intending to be bound by theory, it is believed that as the number of chain branches attached to the core compound increases, the length of each chain branch may increase to accommodate the steric crowding of the branches and the synthetic inhibitors. It will be understood that the length of the chain branch can also depend on the length and flexibility of the synthetic inhibitor to be coupled to the end of each chain branch. The length of each chain branch may be from 2 to 500 atoms.

In embodiments, each chain branch includes an end group at the end that is distal to the core compound. Each end group independently comprise one of a carboxylic acid group, a primary amine group, a secondary amine, or a click chemistry functional group selected from the group of an alkyne, a tetrazine, an alkene, an azide, a nitrone, and a tetrazole. Each end group can be the same or different. In embodiments, each chain branch of the central compound comprises an azido-(PEG)ₙ-amine, wherein each n is independently in a range of 1-50, 1-40, 1-32, 1-30, 1-23, 1-20 or 3-10, or 1, 2, 3, 4, 5, 6, 7, 8, 9, 10, 11, 12, 13, 14, 15, 16, 17, 18, 19, 20, 21, 22 or 23. In embodiments, each n is independently in a range of 1-23. In embodiments, each n is independently in a range of 1 to 20. In embodiments, each n is independently in a range of 3-10. In embodiments, each n is 7.

In embodiments, the central compound comprises 2-6 chain branches, and each chain branch comprises an azido-PEG7-amine. As used herein, the term “PEG #” is used to indicate the number of ethylene glycol (EG) groups. For example, “PEG7” refers to a chain of 7 EG groups. In embodiments, the central compound comprises 1,3,5-benzenetricarboxylate having three chain branches, each chain branch comprising an azido-PEG7-amine coupled to one of the three carboxyl groups of the 1,3,5-benzenetricarboxylate core.

A non-limiting example of the central compound has a chemical structure according to Formula 20 as shown herein below.

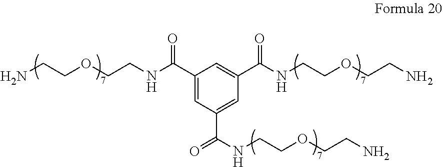

In embodiments, the multi-functional linker comprises: a central compound having a core and two chain branches, the first and second chain branches each having an end group R_{end1} and R_{end2}, respectively. The end groups are each a carboxylic acid, a primary amine, a secondary amine, or a click chemistry functional group selected from the group of an alkyne, a tetrazine, an alkene, an azide, a nitrone, and a tetrazole. In embodiments, the multi-functional linker comprises a central compound having a core and three chain branches, the first, second and third chain branches, each of which has an end group R_{end1}, R_{end2}, and R_{end3} respectively. The end groups are each individually a carboxylic acid, a primary amine, a secondary amine, or a click chemistry functional group selected from the group of an alkyne, a tetrazine, an alkene, an azide, a nitrone, and a tetrazole. In embodiments, the end groups are each individually a carboxylic acid, a primary amine, or a secondary amine.

In embodiments, the central compound comprises a 1,3,5-benzenetricarboxylate core and three chain branches each comprising an azido-(PEG)ₙ- group attached through one of the carboxyl groups of the core, and having a structure according to Formula P as shown herein below, wherein each n is in a range of 1-23, and wherein —R_{end1}, —R_{end2}, and —R_{end3} are the same or different, each of which is independently an end group selected from the group of a carboxylic acid, a primary amine, a secondary amine, or a click chemistry functional group selected from the group of an alkyne, a tetrazine, an alkene, an azide, a nitrone, and a tetrazole:

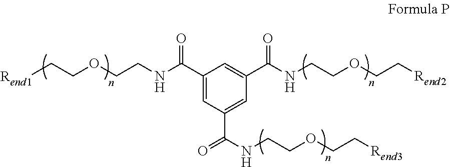

In embodiments, each end group (—R_{end1}, —R_{end2}, and —R_{end3}) is or comprises a carboxylic acid, a primary amine, or a secondary amine. In embodiments, each end group (—R_{end1}, —R_{end2}, and —R_{end3}) is or comprises a primary amine. In embodiments, each end group (—R_{end1}, —R_{end2}, and —R_{end3}) is or comprises a click chemistry functional group selected from the group of an alkyne, a tetrazine, an alkene, an azide, a nitrone, and a tetrazole. In embodiments, each n is independently from 1-50, 1-30, 1-20, 1-10, 3-10, or 3-8. In embodiments, each n is 7.

In general, each branch of the multifunctional linker is or is capable of being coupled to an inhibitor. Generally, each inhibitor can independently be any inhibitor that permanently inactivates an enzyme, protein, antibody fragment, fusion protein, or peptide, such that no other inhibitor-enzyme (protein, antibody fragment, fusion protein, or peptide) complex can form. For example, the inhibitor can be a synthetic inhibitor of the disclosure. Alternative inhibitors are known in the art and suitable inhibitors include, but are not limited to p-nitrophenyl phosphonate (pNPP) inhibitors, chloro-pyrimidine (CP) inhibitors, haloalkane inhibitors, benzylcytosine inhibitors, and benzylguanine inhibitors, for inhibiting, for example, cutinase, SnapTag, HaloTag, or ClipTag.

In embodiments, the multi-functional linker can comprise a first chain branch having first inhibitor coupled thereto, wherein the first inhibitor is a synthetic inhibitor of the disclosure. In embodiments, the multi-functional linker comprises at least one chain branch having a synthetic inhibitor of the disclosure coupled to the linker through the end group of the chain branch that is distal from the core.

The multi-functional linker can comprises at least one inhibitor, at least 2 inhibitors, at least 3 inhibitors, or 2-20, 2-10, 2-8, 2-6, 3-6, 3-5, or 3 inhibitors. In embodiments, the number of the inhibitors and the number of chain branches are the same. That is, the end group of each chain of the multi-functional linker is coupled to an inhibitor. In embodiments, the number of inhibitors and the number of chain branches of the multi-functional linker are not the same, such that the multi-functional linker includes chain branches that have end groups selected from the group of a carboxylic acid, a primary amine, a secondary amine, or a click chemistry functional group selected from the group of an alkyne, a tetrazine, an alkene, an azide, a nitrone, and a tetrazole. The at least 2 inhibitors may be the same or different from each other. In embodiments, the at least two inhibitors are different from each other and one inhibitor is a synthetic inhibitor of the disclosure. In embodiments, the at least two inhibitors are the same and both inhibitors are a synthetic inhibitor of the disclosure. In embodiments, the at least two inhibitors comprise at least one of a synthetic inhibitor having a structure according to Formula G, Formula H, or Formula I. In embodiments, the at least two inhibitors comprise at least one of 4-(6-((4-((fluorosulfonyl)oxy)phenyl)ethynyl)-4,4-dimethyl-3,4-dihydroquinolin-1(2H)-yl)-4-oxohutanoic acid having the structure of Formula 10. In embodiments, the at least two inhibitors may comprise one or more of a synthetic inhibitor having a structure according to any of Formula K, Formula L, Formula M, Formula N, and Formula 0, or an inhibitor selected from ap-nitrophenyl phosphonate (pNPP) inhibitor, a chloro-pyrimidine (CP) inhibitor, a haloalkane inhibitor, a benzylcytosine inhibitor, and a benzylguanine inhibitor. In embodiments, the at least two inhibitors are independently selected from a synthetic inhibitor have a structure according to any of Formula 10, Formula K, Formula L, Formula M or Formula N, and an inhibitor selected from an arylfluorosulfate inhibitor, an arylsulfonyl fluoride inhibitor, ap-nitrophenyl phosphonate (pNPP) inhibitor, a chloro-pyrimidine (CP) inhibitor, a haloalkane inhibitor, a benzylcytosine inhibitor, or a benzylguanine inhibitor.

In embodiments, the multi-functional linker comprises at least three chain branches and the first chain branch, second chain branch, and third chain branch are each coupled to an inhibitor. In embodiments, the three inhibitors can be the same or different from each other. In embodiments, at least one of the three inhibitors is a synthetic inhibitor of the disclosure. In embodiments, at least two of the three inhibitors is a synthetic inhibitor of the disclosure. In embodiments, at least one of the three inhibitors is a synthetic inhibitor having a structure according to Formula G, Formula H, or Formula 1. In embodiments, at least two of the three inhibitors are a synthetic inhibitor having a structure according to Formula G, Formula H, or Formula I. In embodiments, all three of the at least three inhibitors are a synthetic inhibitor having a structure according to Formula G, Formula H, or Formula L In embodiments, all three of the at least three inhibitors in independently selected from the group of 4-(6-((4-((fluorosulfonyl)oxy)phenyl)ethynyl)-4,4-dimethyl-3,4-dihydroquinolin-1(2H)-yl)-4-oxobutanoic acid having a chemical Formula 10, a synthetic inhibitor having a structure according to any of Formula K, Formula L, Formula M, Formula N or Formula 0, an arylfluorosulfate inhibitor, an arylsulfonyl fluoride inhibitor, ap-nitrophenyl phosphonate (pNPP) inhibitor, a chloro-pyrimidine (CP) inhibitor, a haloalkane inhibitor, a benzylcytosine inhibitor, and a benzylguanine inhibitor.

In embodiments, the multi-functional linker has a chemical structure according to Formula Q:

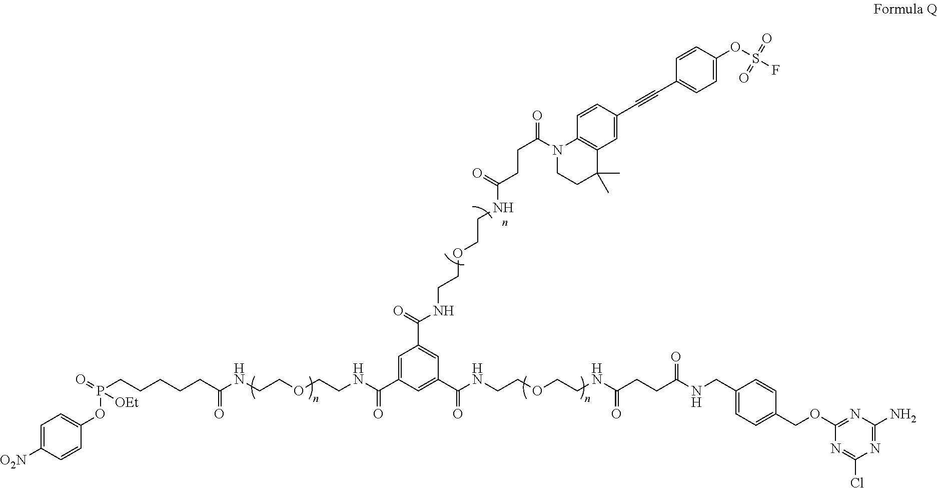

wherein n is in a range of 1-50, 1-40, 1-30, 1-23 or 1-20 In embodiments n is in a range of 1-10, 3-10, or 3-8. In embodiments, n is 7. In embodiments, the multi-functional linker is Tri-EG(7,7,7)—(R,CP,pNPP) having a chemical structure according to Formula 17:

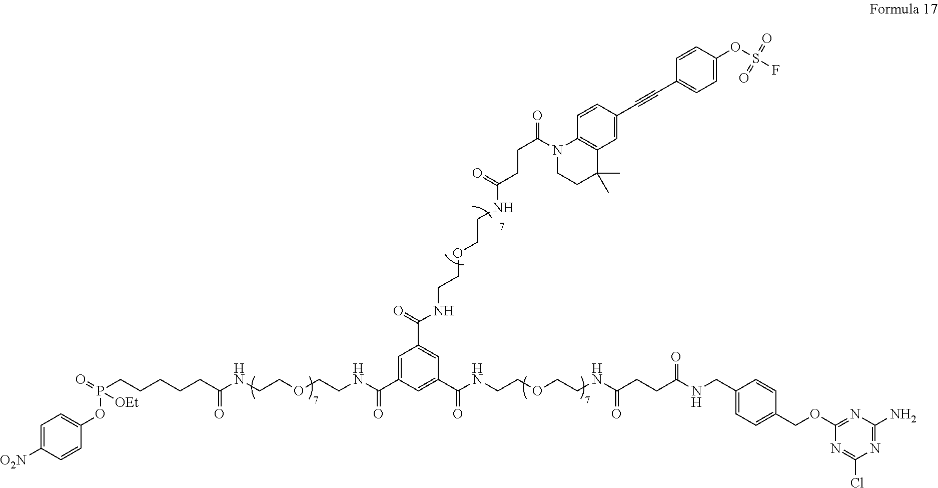

### Metamolecules

The present disclosure further provides a megamolecule comprising: at least one multi-functional linker of the disclosure coupled to at least one bio-matter. As used herein, a “bio-matter” can include any enzyme, protein, antibody fragment, fusion protein, or peptide that has an active site that can couple to an inhibitor or a synthetic inhibitor of the disclosure. In embodiments, the bio-matter comprises one of a protein, a fusion protein, an enzyme, an antibody fragment, or a peptide. In embodiments, the bio-matter comprises an active site. In embodiments, the bio-matter comprises an active enzyme. In embodiments, the bio-matter comprises a protein having an active enzyme. As used herein, the term “active site” refers to the amino acid residues that can form permanent covalent bonds or temporary bonds with a substrate (binding site) and/or residues that catalyze a reaction of that substrate (catalytic site). The active site usually consists of three to four amino acids, while other amino acids control the tertiary structure of the enzyme, protein, antibody fragment, fusion protein, or peptide. As used herein and unless specified otherwise, “active enzyme” refers to an enzyme comprising an active-site residue that can couple to a functional group specific for irreversibly inhibiting the active-site residue. As used herein, the term “active enzyme” and the term “reactive enzyme” are used interchangeably.

The active enzyme may comprise an active site (or a binding pocket). As used herein, the term “active site” and the term “reactive site” are used interchangeably. The synthetic inhibitor can be specific for irreversibly inhibiting the bio-matter at the active site (such as the one or more specific amino acids residues) of an active enzyme by a covalent bond, an ionic bond, and/or a hydrogen bond which is formed by a reaction between a functional group on the inhibitor and the one or more amino acid residues at the active site. The active enzyme may further comprise one or more of additional amino acids residues proximal to the active site amino acid residue to activate, facilitate or accelerate the irreversible inhibition of active enzyme by the synthetic inhibitor. As used herein, the term “proximal” with respect to the distance of an amino acid residue to an active site amino acid residue of a protein refers to any amino acid residue(s) that are located close, e.g., within 5 angstroms of distance to the active site amino acid residues in the active binding pocket of the active enzyme. Proximal amino acid residues can facilitate the binding between an inhibitor with the active site of the protein. For example, CrabTag has an active site tyrosine (tyr) residue which binds with an arylfluorosulfate electrophile (inhibitor). The tyrosine residue binds to the inhibitor through a sulfur fluoride exchange (SuFEx) click reaction, which can be activated by two proximal arginine residues (Arg112 and Arg133) that electrostatically stabilize the reactive sulfur fluoride bond through the formation of a sulfate ester, lowering the pKa of the nucleophile and accelerating the inhibition reaction. In this example, the Arg112 and Arg133 residues are considered proximal to the active site tyrosine residue as they are within 5 angstroms of distance to the tyrosine residue in the active binding pocket of the CrabTag. In embodiments, the active enzyme is a human derived enzyme, such as a cellular retinoic acid binding protein II (CRABP2) (CrabTag).

In embodiments, the bio-matter is or comprises an antibody fragment. Non-limiting examples of antibody fragments include anti-extracellular matrix (anti-ECM), Fibronectin; anti-EV, CD63; anti-tumor, EGFR, DR5, GD2, GPC2, HER2 (5 variants each with different binding affinities for HER2); anti-macrophage, MMR; anti-NK cell, CD16; anti-T cell, CTLA4; anti-myeloid (immune) compartment targeting, CD33, PDL1; anti-vascular, B7-H3, and anti-serum, Albumin.

The bio-matter is generally coupled to the megamolecule, through the inhibitor that is coupled to an end group of a chain branch of a multifunctional linker. In embodiments, the inhibitor is a synthetic inhibitor of the disclosure. In general, inhibitors and synthetic inhibitors of the disclosure include a functional group specific for irreversibly inhibiting or coupling to the bio-matter from a reaction between the functional group and the active site of the bio-matter. In embodiments, the functional group comprises a fuorosulfate group or an arylfulsulfate group which enables sulfur fluoride exchange click chemistry to specifically, selectively, and irreversibly inhibit cellular retinoic acid binding protein II (CRABP2; CrabTag).

In embodiments, the megamolecule of the disclosure can include one multi-functional linker of the disclosure having at least one bio-matter coupled thereto. In embodiments, the megamolecule of the disclosure can include two or more multi-functional linkers, for example, 2, 3,

Megamolecules including two or more multi-functional linkers can be prepared using, for example, fusion proteins having two active enzymes, wherein one active enzyme is coupled to an inhibitor on a first multi-functional linker and the other active enzyme is coupled to an inhibitor on a second multi-functional linkers. Additional multi-functional linkers and fusion proteins can be used in the same way to prepare megamolecules that are networks of multi-functional linkers. Examples of megamolecules and megamolecules that are networks of multi-functional linkers are shown in FIG. 20. FIG. 20 illustrates different types of megamolecules, represented as Configuration 16, Configuration 18, Configuration 19, Configuration 30, Configuration 31, Configuration 32, and Configuration 33. The central grey portion represents the multi-functional linker, the red portion represents the CRABP2 (CrabTag), the blue portion represents cutinase, the yellow portion represents SnapTag, and the green portion represents a fluorescein tag.

In embodiments, the megamolecule can comprise at least 2 bio-matters comprising a first bio-matter and a second bio-matter; and a multi-functional linker, wherein the multi-functional linker comprises a central compound having a core, and at least two chain branches comprising a first chain branch and a second chain branch each of which is coupled to the core; and at least two inhibitors comprising a first inhibitor coupled to the first chain branch, and a second inhibitor coupled to the second chain branch, wherein the first inhibitor specifically and irreversibly inhibits the first bio-matter, and the second inhibitor specifically and irreversibly inhibits the second bio-matter, and wherein the megamolecule has a specific-defined structure and a molecular weight in a range of about 1.0 KDa to about 10.0 MDa. The first and/or second inhibitors can be synthetic inhibitor of the disclosure. The coupling of the first synthetic inhibitor to the first chain branch, and the second synthetic inhibitor to the second chain branch may be through a covalent bond, an ionic bond and/or a hydrogen bond. The coupling of each of the first and second chain branch to the core can be through a covalent bond, an ionic bond and/or a hydrogen bond.

In embodiments, the megamolecule can comprise: two or more multi-functional linkers; and multiple bio-matters, each bio-matter coupled to one of the inhibitors of the multi-functional linker through a covalent bond, an ionic bond and/or a hydrogen bond, preferably a covalent bond.

In embodiments, wherein megamolecules include at least two bio-matters, each of the at least two bio-matters independently comprises a protein, a fusion protein, an enzyme, an antibody fragment, or a peptide. In embodiments, the at least two bio-matters each comprise an enzyme which may be a specific active enzyme. In embodiments, the at least two bio-matters each comprise a protein having a specific active enzyme. The active enzymes each may comprise an active site including one or more active site amino acid residues. The active enzymes each may further comprise one or more additional amino acid residues proximal (within 5 angstroms of distance) to the one or more active site amino acid residues which can facilitate, enable or accelerate the inhibition reaction between the synthetic inhibitor and the corresponding enzyme (the enzyme-inhibitor pair).

In embodiments wherein the megamolecules include multiple proteins, the multiple proteins may each have an active enzyme independently selected from the group of CRABP2 (CrabTag), cutinase, SnapTag, HaloTag and ClipTag.

In embodiments, the megamolecule can include at least one multi-functional linkers, the multi-functional linkers including multiple inhibitors (e.g., 2, 3, 4, 5, 6, or more), the multiple inhibitors each independently selected from the group of a fluorosulfate inhibitor, an arylfluorosulfate inhibitor, a sulfonyl fluoride inhibitor, an arylsulfonyl fluoride inhibitor, a chloro-pyrimidine inhibitor, a p-nitrophenyl phosphonate (pNPP) inhibitor, an O⁶-benzylguanine inhibitor, a benzylcytosine inhibitor, an α-haloalkane inhibitor, a haloaromatic inhibitor, a beta-lactam inhibitor (or derivative thereof such as clavulanic acid or derivative thereof), an aglycone inhibitor, a hydroxamic acid-benzophenone inhibitor, a cognate oriT oligonucleotide sequence inhibitor, and an inhibitor having a structure according to Formula 10, Formula 11, Formula J, Formula K, Formula L, Formula M, Formula N, or Formula O.

In the megamolecules of the disclosure, each of the multiple active enzymes is selectively coupled to its pair synthetic inhibitor. As used herein, the term “enzyme-inhibitor pair” refers to an inhibitor and an enzyme, wherein the inhibitor specifically and selectively inhibits the enzyme at an active site of the enzyme. Non-limiting enzyme-inhibitor pairs include CrabTag-arylfluorosulfate inhibitor pair, cutinase-pNPP inhibitor pair, SnapTag-CP inhibitor pair, HaloTag-haloalkane inhibitor pair, and ClipTag-benzylcytosine inhibitor pair, SnapTag-benzylguanine inhibitor pair, beta-lactamase-beta-lactam inhibitor pair, glycosidase-aglycone inhibitor pair, matrix metalloproteinase-hydroxamic acid-benzophenone inhibitor pair, relaxase domain of type I DNA topoisomerases-cognate oriT oligonucleotide inhibitor pair, cytoplasmic protein tyrosine kinase domain-cysteine-reactive ATP-binding site inhibitor pair, alkaline phosphatase-quinone methide inhibitor pair, alkaline phosphatase-halophophonic acid inhibitor pair, protein tyrosine-phsophatase-formylchromone inhibitor pair, protein-tyrosine-phosphatase-halophosphonic acid inhibitor pair, glucosidase-aglycone inhibitor pair, N-6 adenine-specific DNA methylase-adenosine inhibitor pair, N(4)-cytosine-specific DNA methylase-cytosine inhibitor pair, mutants of haloalkane dehalogenase-haloalkane inhibitor pair, mutants of haloalkane dehalogenase-haloaromatic compound inhibitor pair, gelatinase B-thiirane inhibitor pair, gelatinase B-hydroxamic acid inhibitor pair, gelatinase A-thiirane inhibitor pair, gelatinase A-hydroxamic acid inhibitor pair, fatty acid amide hydrolase-alpha-ketoxazole inhibitor pair, esterase-phosphonate inhibitor pair, esterase-carbamate inhibitor pair, and methionine aminopeptidase-beloranib inhibitor pair. In embodiments, the enzyme-inhibitor pair is selected from the group of CrabTag-arylfluorosulfate inhibitor pair, cutinase-pNPP inhibitor pair, SnapTag-CP inhibitor pair, HaloTag-haloalkane inhibitor pair, and ClipTag-benzylcytosine inhibitor pair, SnapTag-benzylguanine inhibitor pair, beta-lactamase-beta-lactam inhibitor pair, glycosidase-aglycone inhibitor pair, alkaline phosphatase-quinone methide inhibitor pair, alkaline phosphatase-halophosphonic acid inhibitor pair, protein tyrosine-phsophatase-formylchromone inhibitor pair, and glucosidase-aglycone inhibitor pair. In embodiments, the enzyme-inhibitor pair is selected from the group of CrabTag-arylfluorosulfate inhibitor pair, cutinase-pNPP inhibitor pair, SnapTag-CP inhibitor pair, HaloTag-haloalkane inhibitor pair, and ClipTag-benzylcytosine inhibitor pair, and SnapTag-benzylguanine inhibitor pair. Any of the fluorosulfate inhibitor, arylfluorosulfate inhibitor, sulfonyl fluoride inhibitor, arylsulfonyl fluoride inhibitor, and an inhibitor having a structure according to Formula 10, Formula 11, Formula J, Formula K, Formula L, Formula M, Formula N, and Formula O can be coupled to CRABP2 (CrabTag) to form an enzyme-inhibitor pair. Each of the active enzymes may have an active site (binding pocket) to react/couple/complex with the complementary functional group of its paired synthetic inhibitor.

In embodiments, the synthetic inhibitors may comprise one or more of an arylfluorosulfate inhibitor specific for irreversibly inhibiting CRABP2 (CrabTag) through covalent bonding using sulfur fluoride exchange click chemistry, a p-nitrophenyl phosphonate (pNPP) inhibitor specific for irreversibly inhibiting cutinase through covalent bonding, a chloro-pyrimidine (CP) inhibitor specific for irreversibly inhibit SnapTag through covalent bonding. In embodiments, the multiple active enzymes may comprise one or more of CRABP2 (CrabTag), cutinase, and SnapTag.

The CRABP2 has an active site tyrosine (tyr) residue. The irreversible or covalent inhibition of CRABP2 is at its active site tyrosine residue, by an arylfluorosulfate electrophile of the synthetic inhibitor. This sulfur fluoride exchange (SuFEx) click reaction is activated by two proximal arginine residues (Arg112 and Arg133) on the CRABP2 that electrostatically stabilizes the reactive sulfur fluoride bond through the formation of a sulfate ester, lowering the pKa of the nucleophile and accelerating the inhibition reaction.

The megamolecule assemblies of the present disclosure thus can have precisely-defined specificity, orientation, and stoichiometry of protein domains within atomically precise nanostructures of the megamolecule.

In embodiments, the first bio-matter comprises a first protein having a first active enzyme with a first active site which includes a first active site amino acid residue, and the second bio-matter comprises a second protein having a second active enzyme with a second active site which includes a second active site amino acid residue.

In embodiments, the first synthetic inhibitor comprises a first functional group and a fourth functional group, and the second synthetic inhibitor comprises a second functional group and a fifth functional group.

In embodiments, the first synthetic inhibitor is specific for irreversibly inhibiting the first protein at the first active site having the first active site amino acid residue by a covalent bond derived from a reaction between the first functional group and the first active site amino acid residue. The first active enzyme may further comprise one or more of additional amino acids residues proximal (within 5 angstroms of distance) to the first active site amino acid residue to activate, facilitate or accelerate the irreversible inhibition of the first active enzyme by the corresponding first synthetic inhibitor (the first enzyme-inhibitor pair).

In embodiments, the second synthetic inhibitor is specific for irreversibly inhibiting the second protein at the second active site amino acid residue of the second active enzyme by a covalent bond derived from a reaction between the second functional group and the second active site amino acid residue. The second active enzyme may further comprise one or more additional amino acids residues proximal (within 5 angstroms of distance) to the second active site amino acid residue to activate, facilitate or accelerate the irreversible inhibition of the second active enzyme by the second synthetic inhibitor (the second enzyme-inhibitor pair).

In embodiments, the first enzyme is an active enzyme comprising a first terminus, the second enzyme is an active enzyme comprising a second terminus, wherein the first synthetic inhibitor has a first functional group, the second synthetic inhibitor has a second functional group, wherein the inhibition of the first enzyme by the first synthetic inhibitor is through a covalent bond derived from a reaction between the first functional group and the first terminus of the first enzyme, and the inhibition of the second enzyme by the second synthetic inhibitor is through a covalent bond derived from a reaction between the second functional group and the second terminus. The first terminus comprises the first active site having the first active site amino acid residue and the first additional one or more amino acid residues proximal to the first specific active site amino acid residue. The second terminus comprises the second active site with the second specific active site amino acid residue and the second additional one or more amino acid residues proximal to the second specific active site amino acid residue.

In embodiments, the first chain branch has a first end group and the second chain branch has a second end group. In embodiments, the first synthetic inhibitor has a fourth functional group and the second synthetic inhibitor has a fifth functional group. In embodiments, the first synthetic inhibitor is coupled to the first end of the first chain branch through a covalent bond, an ionic bond and/or a hydrogen bond. In embodiments, the first synthetic inhibitor is coupled to the first end of the first chain branch through a covalent bond between the fourth functional group and the first end of the first chain branch. In embodiments, the coupling of the first synthetic inhibitor to the first chain branch is through a covalent bond derived from a reaction between the fourth functional group with the first end group.

In embodiments, the second synthetic inhibitor is coupled to the second end of the second chain branch through a covalent bond, an ionic bond and/or a hydrogen bond. In embodiments, the second synthetic inhibitor is coupled to the second end group of the second chain branch through a covalent bond between the fifth functional group and the second end group f the second chain branch. In embodiments, the coupling of the second synthetic inhibitor to the second chain branch is through a covalent bond derived from a reaction between the fifth functional group and the second end group.

In embodiments, the first and second end groups each independently comprise one of a carboxylic acid group, a primary amine group, and a secondary amine group.

In embodiments, the fourth and fifth functional groups each independently is selected from or comprise one or more of a carboxylic acid group, 4-oxobutanoic acid (—CO—CH₂CH₂—COOH), an activated carboxylic acid group, a functional group having a structure according to formula (—CO—R₆—COOH), an amide group, an amide containing functional group having a structure according to formula (—CO—R₆—CO—NH—(PEG)ₙ-), a NHS ester group, an alcohol group, a fluorophore or fluorescein tag, a click chemistry molecule group including BCN, TCO, tetrazine, DBCO and azide, a primary amine group, a secondary amine group, and a combination thereof, wherein the —R₆— can be an alkyl having up to 20 carbons or 1 to 6 carbons, or an aryl group, wherein n is in a range of 1 to 50, 1 to 40, 1 to 32, 1 to 30, 1 to 23, such as 1 (a single ethylene glycol (EG) group), 3, 4, 6, 7, 9, 10, 11, 12, 23 or 32. The -(PEG)ₙ- may have a molecular weight up to 20K, such as EG 456 (20 k).

Non-limiting examples of the amide group include one of —CO—R₆—CO—NH—(PEG)ₙ-BDCO, —CO—R₆—CO—NH—(PEG)ₙ-azide, and —CO—R₆—CO—NH—(PEG)ₙ-TCO.

In embodiments, each of the fourth and fifth functional groups independently comprises one of the carboxylic acid, —CO—CH₂CH₂—COOH, —CO—R₆—COOH, —CO—R₆—CO—NH—(PEG)ₙ-, and activated carboxylic acid.

In embodiments, each of the fourth and fifth functional groups independently comprises one of the carboxylic acid, —CO—CH₂CH₂—COOH, —CO—R₆—COOH, and activated carboxylic acid.

In embodiments, the central compound comprises 3 chain branches, the first and second chain branches and a third chain branch having a third end group. In embodiments, the at least 2 synthetic inhibitors comprises 3 synthetic inhibitors, the first and second synthetic inhibitors and a third synthetic inhibitor which has a third functional group. In embodiments, the at least 2 enzymes comprises 3 enzymes, the first and second enzymes and a third enzyme. In embodiments, the third synthetic inhibitor has a sixth functional group coupled the third end group of the third chain branch. In embodiments, the third synthetic inhibitor has a third functional group irreversibly inhibiting or coupled to the third enzyme at its active site through a covalent bond, an ionic bond, and/or a hydrogen bond. The active site of the third enzyme comprises one or more active site amino acid residues which reacts with the third functional group of the third synthetic inhibitor to form the covalent bond, an ionic bond, and/or a hydrogen bond. The third enzyme may comprise one or more additional amino acid residues to activate, facilitate or accelerate the inhibition reaction between the third synthetic inhibitor and the active site of the third enzyme.

In embodiments, the three enzymes are each independently selected from the group of CRABP2 (CrabTag), cutinase, SnapTag, HaloTag and ClipTag. The three enzymes can be the same or different from each other. Each of the three enzymes can be part of its corresponding specific protein or fusion protein.

Other bio-matters can also be assembled to the megamolecule. The bio-matters include those pertaining to cancer, cancer treatment or diagnose, such as yeast Cytosine deaminase, and antibody fragments. Non-limiting examples of the antibodies include anti-extracellular matrix (anti-ECM), Fibronectin; anti-EV, CD63; anti-tumor, EGFR, DR5, GD2, GPC2, HER2 (5 variants each with different binding affinities for HER2); anti-macrophage, MMR; anti-NK cell, CD16; anti-T cell, CTLA4; anti-myeloid (immune) compartment targeting, CD33, PDL1; anti-vascular, B7-H3; and anti-serum, Albumin.

The purpose of the megamolecule assembly is to enhance selectivity of a target through generation of a highly avid structure with precise synthesis of the megamolecule scaffold with precisely defined homogeneous structure.

In embodiments, the first functional group of the first synthetic inhibitor is a fluorosulfate group or an arylfluorosulfate group, the first enzyme is the cellular retinoic acid binding protein II (CRABP2) (CrabTag), and the irreversibly inhibition of the CRABP2 by the first synthetic inhibitor is through a covalent bonding (the first enzyme-inhibitor pair). The covalent bond is derived from a reaction between the fluorosulfate or arylfluorosulfate group and the first active site of the first enzyme using sulfur fluoride exchange click chemistry.

In embodiments, the second and third functional groups are each independently selected from the group of a p-nitrophenyl phosphonate (pNPP) group specific for irreversibly inhibiting cutinase through a covalent bond, a chloro-pyrimidine (CP) group specific for irreversibly inhibit SnapTag through a covalent bond, a haloalkane group specific for irreversibly inhibiting HaloTag, a benzylcytosine group specific for irreversibly inhibiting ClipTag, and a benzylguanine group specific for irreversibly inhibiting SnapTag. In embodiments, the first, second and third functional groups may be the same or different from each other.

In embodiments, the central compound has a chemical Formula 20 as shown herein above, and the first, second and third end groups each are a primary amine group.

In embodiments, the first synthetic inhibitor is a synthetic retinoid inhibitor 4-(6-((4-((fluorosulfonyl)oxy)phenyl)ethynyl)-4,4-dimethyl-3,4-dihydroquinolin-1(2H)-yl)-4-oxobutanoic acid having a chemical Formula 10 as shown herein above. The first functional group is an arylfluorosulfate group, and the fourth functional group is carboxylic acid group.

In embodiments, the first synthetic inhibitor is a synthetic retinoid inhibitor having a chemical Formula 11 (Bi-EG(11)-(CrabTagLigand, Fluorescein)) as discussed herein above. This synthetic inhibitor has a fluorophore which can be used to characterize the kinetics for the reaction of this covalent inhibitor and CRABP2 (Crabtag). In embodiments, the first synthetic inhibitor may be one of a synthetic inhibitor having a structure according to Formula 10, Formula 11, Formula J, Formula K, Formula L, Formula M, Formula N and Formula 0, as discussed herein above.

In embodiments, the megamolecule remains at least 95 wt. % intact after storage at room temperature in PBS solution at a concentration of about 1 μM for up to 2 weeks and thus is stable.

In embodiments, the first enzyme is a human-derived enzyme, such as a human-derived cellular retinoic acid binding protein II (CRABP2) (CrabTag) which comprises a first terminus having an first active site amino acid residue. The first active site amino acid residue is a tyrosine (tyr) residue. In embodiments, the first terminus (of the first enzyme) further comprises two arginine residues, Arg 112 and Arg 133 proximal (within 5 angstroms of distance) to the tyrosine (tyr) residue. The two arginine residues electrostatically stabilize the reactive sulfur fluoride bond through the formation of a sulfur ester and thus accelerating the inhibition reaction of the first active enzyme by the first synthetic inhibitor.

In embodiments, the second and third enzymes are each independently selected from the group of CRABP2 (CrabTag), cutinase, SnapTag, HaloTag and ClipTag. The second and third enzymes may be the same or different, and may be the same as or different from the first enzyme.

In embodiments, the second and third synthetic inhibitors are each selected from the group of a synthetic inhibitor having a structure according to Formula 10, Formula 11, Formula J, Formula K, Formula L, Formula M, Formula N and Formula 0, as discussed herein above; a p-nitrophenyl phosphonate (pNPP) inhibitor, a chloro-pyrimidine (CP) inhibitor, a haloalkane inhibitor, a benzylcytosine inhibitor, and a benzylguanine inhibitor.

In embodiments, a non-limiting example of the multi-functional linker is Tri-EG(7,7,7)—(R,CP,pNPP) having a structure according to Formula 17 as shown herein above.

In embodiments, the megamolecule comprises: the multi-functional linker having a structure according to Formula 17 as shown herein above, a first protein having a first active enzyme of CrabTag covalently coupled to the inhibitor of Formula 10 of the multi-functional linker, a second protein comprising a second active enzyme cutinase coupled to the p-nitrophenyl phosphonate (pNPP) inhibitor of the multi-functional linker, and a third protein comprising a third active enzyme SnapTag coupled to the chloro-pyrimidine (CP) inhibitor of the multi-functional linker. The megamolecule has a structure according to Configuration 18 as shown in FIG. 20.

The present disclosure further provides methods of preparing the synthetic inhibitors, the multi-functional linkers and the megamolecules discussed herein above. The methods of preparing the megamolecule assemblies can be accomplished by a one-pot enzyme-inhibitor reaction using cellular retinoic acid binding protein II (CRABP2) and an enzyme building block such as the multiple functional linker discussed herein above.

### Method of Preparing a Synthetic Inhibitor Having a Structure of Formula 10

Another aspect of the disclosure provides a method of preparing a synthetic inhibitor having a structure according to Formula 10, the method comprising: reacting a compound having a structure of Formula 9 (4-(6-((4-hydroxyphenyl)ethynyl)-4,4-dimethyl-3,4-dihydroquinolin-1(2H)-yl)-4-oxobutanoic acid) with [4-(acetylamino)phenyl]imidodisulfuryl difluoride (AISF) in a first solvent to form a first product mixture comprising the synthetic inhibitor having the structure according to Formula 10.

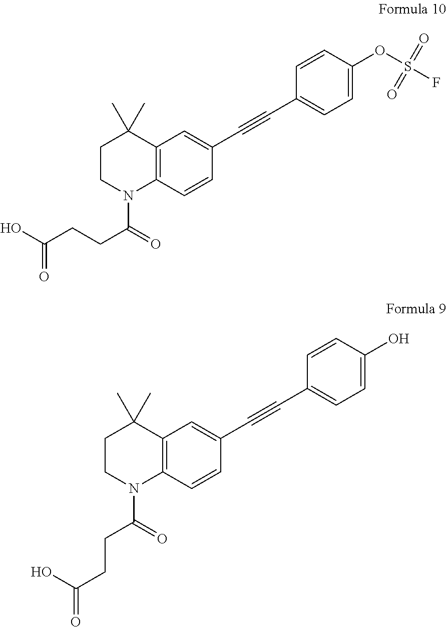 [SMILES: CC1(C)CCN(C(=O)CCC(=O)O)c2ccc(C#Cc3ccc(OS(=O)(=O)F)cc3)cc21 ; CC1(C)CCN(C(=O)CCC(=O)O)c2ccc(C#Cc3ccc(O)cc3)cc21]

In embodiments, the first solvent is tetrahydrofuran (THF). In embodiments, the reacting comprises adding a catalyst during the reacting. In embodiments, the catalyst comprises (1,8-diazabicyclo[5.4.0]undec-7-ene (DBU)). In embodiments, the adding of the 1,8-diazabicyclo[5.4.0]undec-7-ene (DBU) is dropwise over a period of time of about 20-500 seconds, or about 20-60 seconds, or about 30 seconds. In embodiments, the reacting is conducted at room temperature.

In embodiments, the method further comprises: diluting and extracting the first product mixture in ethyl acetate (EtOAc) to form an organic layer; washing the organic layer with 0.5 M HCl (aq.) and subsequently with saturated HNaCO₃ (aq.); acidifying by washing with 0.5 M HCl (aq.) and subsequently washing with brine; drying with Na₂SO₄; concentrating a liquid portion to form a crude mixture; purifying the crude mixture by silica chromatography (10:90 MeOH:DCM); and; concentrating to form the synthetic inhibitor having the structure of Formula 10.

In embodiments, the method further comprises: a process of preparing the compound having the structure of Formula 9, the process comprising saponifying a compound having a structure of Formula 8 (methyl 4-(6-((4-(acetyloxy)phenyl)ethynyl)-4,4-dimethyl-3,4-dihydroquinolin-1(2H)-yl)-4-oxobutanoate).

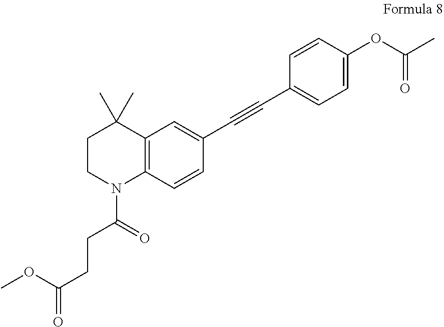 [SMILES: COC(=O)CCC(=O)N1CCC(C)(C)c2cc(C#Cc3ccc(OC(C)=O)cc3)ccc21]

In embodiments, the saponifying comprises: reacting the compound having the structure of Formula 8 with a first base in a second solvent to form a second product mixture comprising the compound having the structure of Formula 9. In embodiments, the second solvent comprises methanol (MeOH) and the first base comprises sodium hydroxide (NaOH). In embodiments, the reacting is conducted at room temperature for a time period in a range of about 30-100 minutes, or about 50-70 minutes, or about 55-65 minutes, or about 60 minutes.

In embodiments, the method further comprises: acidifying the second product mixture using an aqueous 0.5 M hydrochloric acid (HCl) solution; diluting and extracting with ethyl acetate (EtOAc) to form an organic layer; washing the organic layer with water and subsequently with brine; drying with Na₂SO₄; concentrating; purifying by silica chromatography (80:20 EtOAc:Hexanes+1% Acetic acid (AcOH)); washing with toluene; and concentrating to form the compound having the structure of Formula 9.

In embodiments, the method further comprises: a process of preparing the compound having the structure of Formula 8, the process comprising: reacting a compound having a structure of Formula 4 (methyl-4(6-iodo-4,4-dimethyl-3,4-dihydroquinolin-1(2H)-yl)-4-oxobutanoate) with a compound having a structure of Formula 7 (4-ethynylphenyl acetate) to form a third product mixture comprising the compound having the structure of Formula 8:

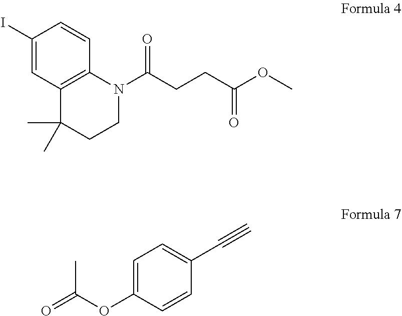 [SMILES: COC(=O)CCC(=O)N1CCC(C)(C)c2cc(I)ccc21 ; C#Cc1ccc(OC(C)=O)cc1]

In embodiments, the reacting is conducted in presence of Pd(PPh₃)₂Cl₂, CuI, a third solvent and a second base. In embodiments, the third solvent comprises dimethylformamide (DMF), and the second base comprises triethylamine (Et₃N). In embodiments, the reacting is conducted at a temperature of about 45-55° C., or about 50° C., for a time period of about 1-4 hours, or about 2.5 hours. In embodiments, a molar ratio of the compound having the structure of Formula 4 to the compound having the structure of Formula 7 is in a range of about 0.5:1 to about 1:2, or about 1:1 to about 1:1.2, or about 1:1.1. In embodiments, the method further comprises: diluting the third product mixture with water at about room temperature; extracting with ethyl acetate (EtOAc); washing with brine to form a liquid mixture; collecting an organic layer of the liquid mixture; drying the organic layer using Na₂SO₄; concentrating; purifying using silica chromatography (25:75 EtOAc:Hexanes); and concentrating to yield the compound having the structure of Formula 8.

In embodiments, the method further comprises: a process of preparing the compound having the structure of Formula 4, the process comprising: reacting a compound having a structure of Formula 3 (6-iodo-4,4-dimethyl-1,2,3,4-tetrahydroquinoline) with methyl 4-chloro-4-oxobutyrate in a fourth solvent to form a fourth product mixture comprising the compound having the structure of Formula 4.

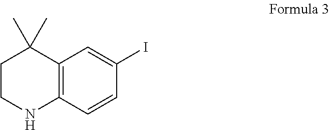

In embodiments, the reacting is conducted in presence of NaHCO₃, and the fourth solvent comprises dichloromethane (DCM). In embodiments, the reacting is conducted at about 10-50° C. or about room temperature for about 6-20 hours or about 12-16 hours. In embodiments, In embodiments, the reacting comprises: dissolving the compound having the structure of Formula 3 in the fourth solvent to form a solution; adding NaHCO₃ to the solution and stirring for about 30 minutes to form a mixture; adding methyl 4-chloro-4-oxobutyrate dropwise to the mixture at about 0-25° C., or about 0-5° C., or about 0° C.; and stirring at about 0-50° C. or about room temperature for about 6-20 hours or about 12-16 hours to form the fourth product mixture. In embodiments, the method further comprises: diluting the fourth product mixture with H₂O to form a liquid mixture; collecting an organic layer of the liquid mixture; washing with 10% Na₂CO₃ (aq.), subsequently with sat. NH₄Cl (aq.), and then with brine; drying with Na₂SO₄; concentrating; and purifying by silica chromatography (30:70 EtOAc:Hexanes) to form the compound having the structure of Formula 4.

In embodiments, the method further comprises: a process of preparing the compound having the structure of Formula 3, the process comprising: reacting a compound having a structure of Formula 2 (6-iodo-4,4-dimethyl-3,4-dihydroquinolin-2(1H)-one) in presence of borane dimethyl sulfide form a fifth product mixture comprising the compound having the structure of Formula 3.

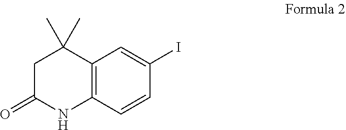

In embodiments, the reacting comprises: dissolving the compound having the structure of Formula 2 in a solvent comprising toluene to form a mixture; dissolving the borane dimethyl sulfide in a solvent comprising tetrahydrofuran (THF) to form a solution; adding the solution to the mixture; and stirring at reflux for about 4-12 hours or about 8 hours to yield the fifth product mixture. In embodiments, the reacting is conducted under nitrogen atmosphere.

In embodiments, the method further comprises: stirring the fifth product mixture for about 20-200 minutes, or about 30 minutes at about room temperature; adding about 10 wt. % Na₂CO₃ (aq.); extracting with ethyl acetate (EtOAc); washing with H₂O and subsequently with brine to form a liquid mixture; collecting an organic layer of the liquid mixture; drying with Na₂SO₄; concentrating; purifying using silica chromatography (10:90 EtOAc:Hexanes+1% triethylamine (Et3N)); and concentrating to form the compound having the structure of Formula 3.

In embodiments, the method further comprises a process of preparing the compound having the structure of Formula 2, the process comprising: reacting a compound having a structure of Formula 1 (N-(4-Iodophenyl)-3-methyl-2-butenamide) with AlCl₃ in a fifth solvent to form a sixth product mixture comprising the compound having the structure of Formula 2;

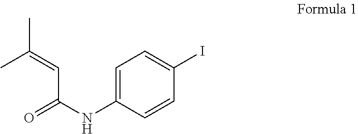

In embodiments, the fifth solvent comprises dichloromethane (DCM). In embodiments, the reacting is conducted under nitrogen atmosphere at about 10-50° C., or about room temperatures, for about 0.1-10 hours, or about 1-5 hours, or about 2.5 hours.

In embodiments, the reacting comprises: admixing the compound having the structure of Formula 1 with AlCl₃ in a container; purging the container with N₂; adding dichloromethane (DCM) to the container; and stirring at about room temperature for about 2.5 fours to form the sixth product mixture. In embodiments, the method further comprises: diluting the sixth product mixture with water and stirring for about 10 minutes; adding 1.25 M NaOH (aq.) to form a solution mixture; collecting an organic layer of the solution mixture; washing the organic layer with water and subsequently with brine; drying with Na₂SO₄; concentrating; and purifying by recrystallizing in EtOH to form the compound having the structure of Formula 2.

In embodiments, the method further comprises a process of preparing the compound having the structure of Formula 1, the process comprising: reacting 4-iodoaniline with 3,3-dimethylacryloyl chloride in a sixth solvent to form a seventh product mixture comprising the compound having the structure of Formula 1.

In embodiments, the sixth solvent comprises pyridine and dichloromethane (DCM). the reacting is at about 0-50° C. or about room temperature for about 6-20 hours or about 12-16 hours.

the reacting comprises: dissolving 4-iodoaniline in dichloromethane (DCM) in a container; adding pyridine to the container and stirring for about 10-200 minutes or about 30 minutes; adding 3,3-dimethylacryloyl chloride to the container dropwise at a temperature in a range of about 0-25° C., or about 0-5° C., or about 0° C.; raising the temperature of the container to about 10-50° C., or about room temperature, and stirring for about 1-50 hours, about 6-20 hours, or about 12-16 hours, to form the seventh product mixture.

In embodiments, the method further comprises diluting the seventh product mixture with water; extracting with ethyl acetate (EtOAc) to form an organic layer; washing the organic layer with 1 M HCl (aq.), subsequently with sat. NaHCO₃ (aq.) and then with brine; drying with Na₂SO₄; concentrating; and purifying by recrystallizing in EtOH to form the compound having the structure of Formula 1.

In embodiments, the method further comprises a process of preparing the compound having the structure of Formula 7, the process comprising: reacting a compound having a structure of Formula 6 (4-((trimethylsilyl)ethynyl)phenyl acetate) with potassium fluoride in a seventh solvent to form an eighth product mixture comprising the compound having the structure of Formula 7.

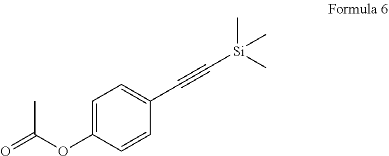 [SMILES: CC(=O)Oc1ccc(C#C[Si](C)(C)C)cc1]

In embodiments, the seventh solvent comprises dimethylformamide (DMF) and water in a volume ratio of about 0.1:1 to about 1:0.1, or about 0.9:1 to about 1:0.9, or about 1:1. In embodiments, the reacting comprises stirring at a temperature in a range of about 10-50° C., or about room temperature for about 10-200 minutes, or about 30-90 minutes, or about 55-65 minutes, or about 60 minutes. In embodiments, the method further comprises diluting the eighth product mixture with water; extracting with EtOAc to form an organic layer; washing the organic layer with brine; drying with Na₂SO₄; concentrating; purifying by silica chromatography (10:90 EtOAc:Hexanes); and concentrating to yield the compound having the structure of Formula 7.

In embodiments, the method further comprises a process of preparing the compound have the structure of Formula 6, the process comprising: reacting a compound having a structure of Formula 5 (4-iodophenyl acetate) with trimethylsilyl-acetylene in a eighth solvent to yield a ninth product mixture comprising the compound having the structure of Formula 6.

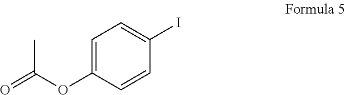

In embodiments, the eighth solvent comprises dimethylformamide (DMF), and the reacting is conducting under nitrogen atmosphere in presence of Pd(PPh₃)₂Cl₂, CuI and a base comprising triethylamine (Et₃N) at about 20-100° C., or about 30-70° C., or about 40-60° C., or about 45-55° C., or about 50° C., for about 0.5-20 hours, or about 1-10 hours, or about 1-5 hours, or about 2-3 hours, or about 2.5 hours.

In embodiments, the method further comprises cooling the ninth product mixture to about room temperature; diluting with water; extracting with ethyl acetate (EtOAc) to form an organic layer; washing the organic layer with brine; drying the organic layer with Na₂SO₄; concentrating; purifying by silica chromatography (10:90 EtOAc:Hexanes); and concentrating to yield the compound having the structure of Formula 6.

In embodiments, the method further comprises dissolving 4-iodophenol in dichloromethane (DCM) in a container; adding triethylamine (Et₃N) to the container and stirring for about 10-200 minutes, or about 20-60 minutes, or about 25-35 minutes, or about 30 minutes; adding acetyl chloride to the container dropwise; stirring at 10-50° C., or about room temperature, for about 0.5-20 hours, or about 1-5 hours, or about 1-3 hours, or about 2 hours to yield the tenth product mixture.

In embodiments, the dissolving, adding the triethylamine (Et3N) and adding the acetyl chloride are each conducted at a temperature of about 0-15° C., or about 0-5° C., or about 0° C.

In embodiments, the method further comprises diluting the tenth product mixture with water to a liquid mixture; collecting an organic layer of the liquid mixture; washing the organic layer with brine; drying with Na₂SO₄; concentrating; purifying by silica chromatography (10:90 EtOAc:Hexanes); and concentrating to yield the compound having the structure of Formula 5.

### Method of Preparing Synthetic Inhibitor Having the Structure of Formula 11

Another aspect of the disclosure provides a method of preparing a synthetic inhibitor have a structure of Formula 11, the method comprising: admixing a synthetic inhibitor have a structure of Formula 10 with 6-[fluorescein-5(6)-carboxamido]hexanoic acid in a container; adding azido-PEG11-amine linker to the container; and reacting to form the synthetic inhibitor having the structure of Formula 11

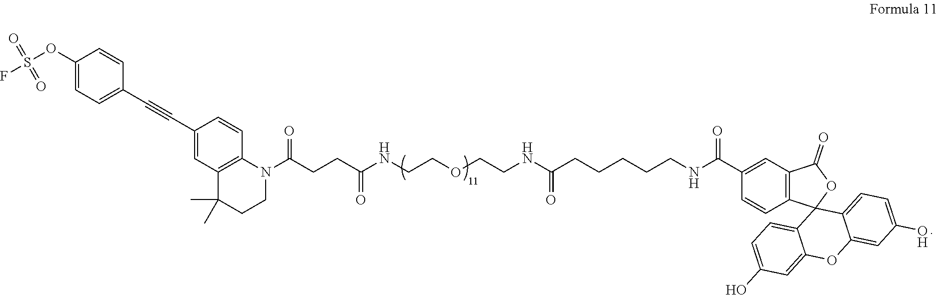

In embodiments, the admixing comprises: dissolving the synthetic inhibitor have the structure of Formula 10 and the -[fluorescein-5(6)-carboxamido]hexanoic acid in a first solvent in the container; adding a second solvent to the container; and stirring for about 0.5-24 hours, or about 1-10 hours, or about 2-6 hours, or about 3-5 hours, or about 3.5-4.5 hours, or about 4 hours. In embodiments, the first solvent comprises 4-(4,6-dimethoxy-1,3,5-triazin-2-yl)-4-methyl-morpholinium chloride (DMTMM) and tetrahydrofuran (THF). In embodiments, the second solvent comprises N-Methylmorpholine (NMM). In embodiments, the method further comprises removing DMTMM, THF and NMM from the container before adding the azido-PEG11-amine linker to the container. In embodiments, the reacting is conducted in a third solvent while stirring for about 6-24 hours, or about 8-20 hours, or about 12-16 hours, or about 16 hours to form the synthetic inhibitor having the structure of Formula 11. In embodiments, the third solvent is dimethylformamide (DMF) and NMM.

The method of preparing a synthetic inhibitor having a structure of Formula 11, comprises: admixing a synthetic inhibitor have a structure according to Formula 10 with 6-[fluorescein-5(6)-carboxamido]hexanoic acid in 4-(4,6-dimethoxy-1,3,5-triazin-2-yl)-4-methyl-morpholinium chloride (DMTMM) and tetrahydrofuran (THF) in a container; adding N-Methylmorpholine (NMM) to the container and stirring for about 0.5-24 hours, or about 1-10 hours, or about 2-6 hours, or about 3-5 hours, or about 3.5-4.5 hours, or about 4 hours; removing DMTMM, THE and N-Methylmorpholine (NMM) from the container; adding dimethylformamide (DMF), azido-PEG11-amine linker and N-Methylmorpholine (NMIM) to the container; reacting while stirring for about 6-24 hours, or about 8-20 hours, or about 12-16 hours, or about 16 hours to form a solution; diluting the solution with 9:1 dimethyl sulfoxide (DMSO):H₂O; purifying the solution on a C18 reversed phase column via HPLC using TFA as the mobile phase additive to form a liquid mixture comprising the synthetic inhibitor having the structure of Formula 11; and lyophilizing the liquid mixture to yield the synthetic inhibitor having the structure of Formula 11.

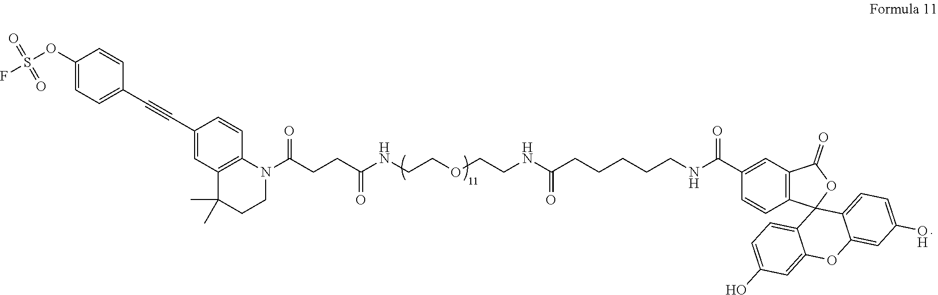

Other synthetic inhibitors having the structures of Formula K, Formula L, Formula M, Formula N, or Formula O each can be similarly prepared by admixing the synthetic inhibitor have the structure of Formula 10 with the corresponding click chemistry molecule of azide, dibenzocyclooctyne (DBCO), bicyclononyne (BCN), trans-cyclooctene (TCO), or tetrazine (Tz) in a container; adding azido-PEG11-amine linker to the container; and reacting in sufficient conditions to form the synthetic inhibitor having the structure of Formula K, Formula L, Formula M, Formula N, or Formula O.

### Method of Preparing a Multi-Functional Linker

Another aspect of the disclosure provides a method of preparing a multi-functional linker of any of claims 12-51, the method comprising: reacting a central compound with a first synthetic inhibitor and a second synthetic inhibitor, wherein the central compound includes a core, a first chain branch and a second chain branch, each of the first and second chain branches is coupled to the core, wherein the first chain branch has a first end group, and the second chain branch has a second end group, wherein the first synthetic inhibitor comprises a first functional group specific for irreversibly inhibiting a first bio-matter, and a fourth functional group, wherein the second synthetic inhibitor comprises a second functional group specific for irreversibly inhibiting a second bio-matter, and a fifth functional group, wherein the reacting comprises reacting the first end group with the fourth functional group to form a bond including a covalent bond, an ionic bond and/or a hydrogen bond through which the first synthetic inhibitor is coupled to the first chain branch, wherein the reacting comprises reacting the second end group with the fifth functional group to form a bond including a covalent bond, an ionic bond and/or a hydrogen bond through which the second synthetic inhibitor is coupled to the second chain branch, and wherein the first and second functional groups are each independently selected from the group of a fluorosulfate (F—SO₂—O—), an arylfluorosulfate (F—SO₂—O—Ar), a sulfonyl fluoride (F—SO₂—), an arylsulfonyl fluoride (F—SO₂—Ar), a p-nitrophenyl phosphonate (pNPP), a chloro-pyrimidine (CP), a haloalkane, a benzylcytosine, and a benzylguanine.

In embodiments, the fourth and fifth functional groups are each independently selected from the group of a carboxylic acid, —CO—CH₂CH₂—COOH, —CO—R₆—COOH, an amide, —CO—R₆—CO—NH—(PEG)ₘ-COOH, an activated carboxylic acid, a n-hydroxysuccinimide (NHS) ester, an alcohol, a click chemistry molecule, a primary amine, and a secondary amine, wherein the —R₆— can be an aryl group or an alkyl having 1 to 6 carbons, and wherein m is in a range of about 1-50, or about 1-30, or about 1-23, or about 1-10.

In embodiments, the click chemistry molecule comprises one of Bicyclononyne (BCN), trans-cyclooctene (TCO), tetrazine (Tz), dibenzocyclooctyne (DBCO) and azide.

In embodiments, wherein the first and second end groups are each independently selected from the group of a carboxylic acid, a primary amine, a secondary amine, the click chemistry molecule, and a n-hydroxysuccinimide (NHS) ester.

In embodiments, the multi-functional linker is a compound having a structure according to Formula 17 (Tri-EG(7,7,7)—(R,CP,pNPP)).

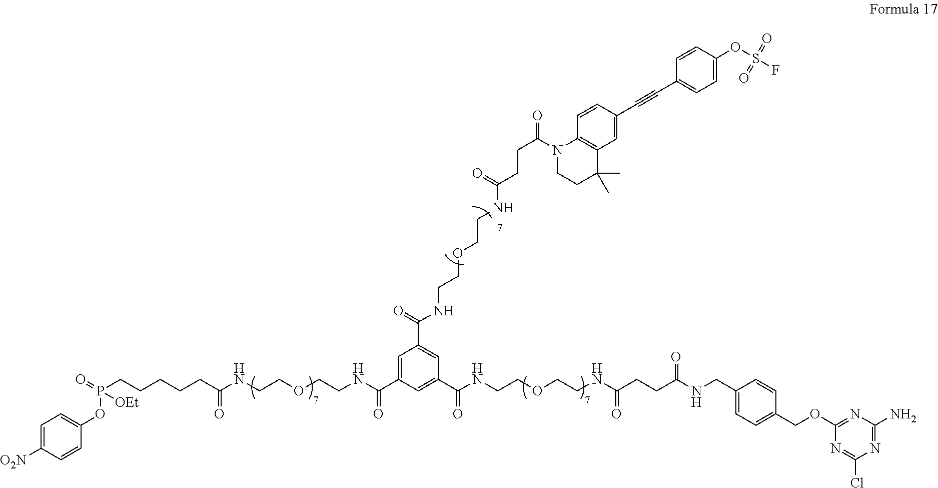

In embodiments, the method comprises a process of preparing a resin having a structure of Resin X1, the process comprising: admixing and swelling 2-chlorotritylchloride resin with N-methylpyrrolidinone for a time period of about 0.1-20 hours, or about 0.5-5 hours, or about 0.5-2 hours, or about 1 hour; adding azido-EG7-amine and diisopropylethylamine (DIEA) to the container; mixing and reacting for about 6-24 hours, or about 8-20 hours, or 12-16 hours, or about 16 hours, to form a mixture; draining the mixture under vacuum to form a reacted resin; washing the reacted resin with dimethylformamide (DMF) and subsequently with dichloromethane (DCM); drying to form an intermediate resin; resuspending the intermediate resin in a mixture of 10% methanol: 90% N-methyl-2-pyrrolidone (NMP); adding DIEA and mixing for about 6-24 hours, or about 8-20 hours, or about 12-16 hours; draining to form a modified resin; washing the modified resin with dimethylformamide (DMF) and subsequently with dichloromethane (DCM); and drying to yield the resin having the structure of Resin X1.

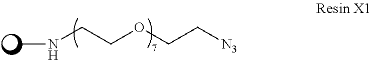

In embodiments, the method further comprises a process of preparing a resin having a structure of Resin X2, the process comprising: treating the Resin X1 with trimethylphosphine in tetrahydrofuran (THF) for about 0.1-24 hours, or about 0.5-20 hours, or about 1-5 hours, or about 2.5-3.5 hours, or about 3 hours; draining to form a treated resin; washing the treated resin with tetrahydrofuran (THF) and drying to form a dried treated resin; treating the dried treated resin with a mixture of 10% water:90% tetrahydrofuran (THF) for about 6-24 hours, or about 8-20 hours, or 12-16 hours, or about 16 hours; washing with tetrahydrofuran (THE), subsequently with dimethylformamide (DMF) and then with dichloromethane (DCM); and drying to yield the resin having the structure of Resin X2 (Amine-EG7-amine protected by the swelled 2-chlorotritylchloride resin).

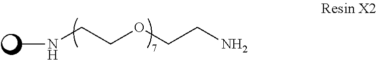

In embodiments, the method further comprises a process of preparing a resin having a structure of Resin X3, the process comprising: swelling the resin having the structure of Resin X2 in N-methyl-2-pyrrolidone (NMP) in the container for about 6-24 hours, or about 8-20 hours, or 12-16 hours, or about 16 hours; adding diethyl 1,3,5-benzenetricarboxylate, hydroxybenzotriazole (HOBT) and 1-ethyl-3-(3-dimethylaminopropyl)carbodiimide (EDC)·HCl and NMP to the container; reacting for about 6-24 hours, or about 8-20 hours, or 12-16 hours, or about 16 hours to form a reacted resin; washing the reacted resin with dimethylformamide (DMF) and subsequently with dichloromethane (DCM); and drying to yield the resin having the structure of Resin X3.

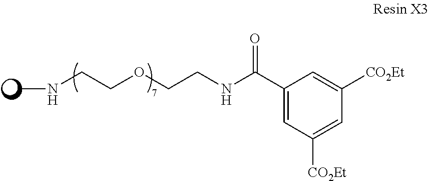

In embodiments, the method further comprises suspending the resin having the structure of Resin X3 in EtOH; adding NaOH aqueous solution; reacting for about 0.1-24 hours, or about 0.5-20 hours, or about 1-5 hours, or about 2.5-3.5 hours, or about 3 hours to saponify the resin having the structure of Resin X3 to form a crude saponified resin; washing the crude saponified resin with water, 10% AcOH, water, dimethylformamide (DMF), and dichloromethane (DCM) sequentially; and drying to yield the saponified resin having the structure of Resin X3.

In embodiments, the method further comprises a process of preparing a resin have a structure of Resin X4, the process comprising: admixing the saponified resin having the structure of Resin X3 with N-methyl-2-pyrrolidone (NMP), hydroxybenzotriazole (HOBT), and EDC·HCl in the container by shaking for about 10-120 minutes, or about 15-100 minutes, or about 20-80 minutes, or about 25-60 minutes, or about 25-35 minutes, or about 30 minutes; adding azido-EG7-amine to the container; and reacting for about 6-24 hours, or about 8-20 hours, or about 12-16 hours, or about 16 hours; draining; washing with dimethylformamide (DMF) and subsequently with dichloromethane (DCM), and drying to yield the resin having the structure of Resin X4.

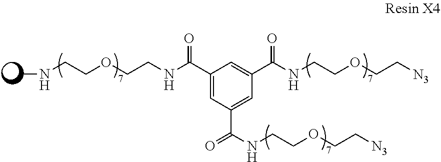

In embodiments, the method further comprises a process of preparing a resin having a structure of Resin X5, the process comprising: treating the resin having the structure of Resin X4 with trimethylphosphine in tetrahydrofuran (THF) for about 0.1-24 hours, or about 0.5-10 hours, or about 1-5 hours, or about 2-4 hours, or about 3 hours; draining; washing with tetrahydrofuran (THF); treating with a 10% water:90% THF mixture for about 6-24 hours, or about 8-20 hours, or about 12-16 hours, or about 16 hours; washing with THF, dimethylformamide (DMF), and dichloromethane (DCM) sequentially; and drying to yield the resin having the structure of Resin X5.

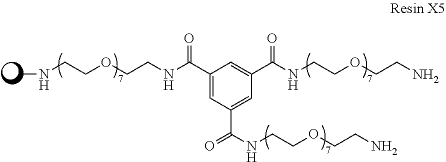

In embodiments, the method further comprises a process of preparing a resin have the structure of Resin X6, the process comprising: swelling the resin having the structure of Resin X5 in N-methyl-2-pyrrolidone (NMP) in the container for about 5-200 minutes, or about 10-100 minutes, or about 20-40 minutes, or about 25-25 minutes, or about 30 minutes; admixing SnapTag inhibitor (CP inhibitor), the cutinase inhibitor (pNPP inhibitor), hydroxybenzotriazole (HOBT), and EDC·HCl and NMP in a second container to form a mixture solution; adding the mixture solution to the container and admixing for about 6-24 hours, or about 8-20 hours, or about 12-16 hours, or about 16 hours; washing with dimethylformamide (DMF) and subsequently with DCM; and drying to yield the resin having the structure of Resin X6.

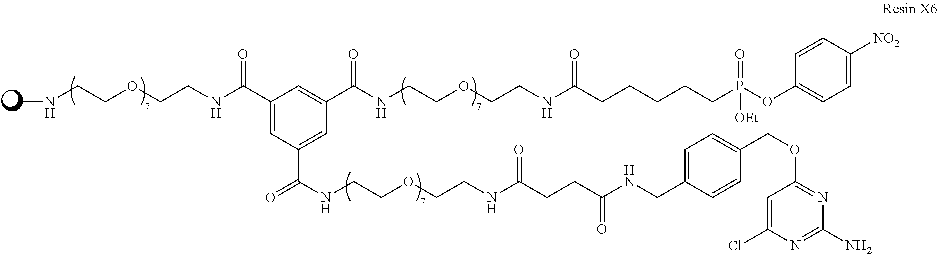

In embodiments, the method further comprises a process of preparing a compound having a structure of Oil X7, the process comprising: admixing the resin having the structure of Resin X6 with a solution having about 5% trifluoroacetic acid (TFA) in dichloromethane (DCM) and reacting for about 0.1-24 hours, or about 0.5-10 hours, or about 1-5 hours, or about 1-3 hours, or about 1.5-2.5 hours, or about 2 hours (to cleave the resin having the structure of Resin X6 to remove the 2-chlorotritylchloride resin); removing excess TFA to yield the compound having the structure of Oil X7.

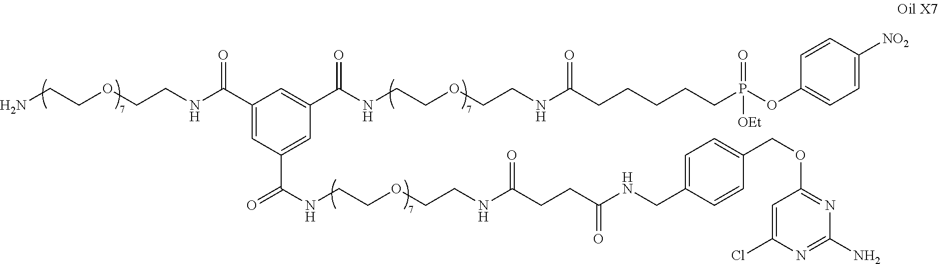

In embodiments, the method further comprises a process of preparing the multi-functional linker having the structure of Formula 17, the process comprising: admixing the compound having the structure of Oil X7 with N-Methylmorpholine (NMM) in the container; admixing a synthetic inhibitor having a structure Formula 10 (CrabTag inhibitor) with dimethylformamide (DMF), hydroxybenzotriazole (HOBT) and EDC HCl in a third container to form a mixture; adding the mixture to the container having the compound having the structure of Oil X7 and N-Methylmorpholine (NMM); reacting for about 6-24 hours, or about 8-20 hours, or about 12-16 hours, or about 16 hours, to form a solution; diluting the solution with a 90%:10% dimethyl sulfoxide (DMSO):water mixture; purifying on a C8 reversed phase column by HPLC; and lyophilizing to yield the multi-functional linker having the structure of Formula 17.

Other multi-functional linkers having the same central compound having the structure of Formula 20 with different combinations of synthetic inhibitors selected from any of the synthetic inhibitors having the structure of Formula 10 or Formula K to Formula 0, ap-nitrophenyl phosphonate (pNPP) inhibitor, a chloro-pyrimidine (CP) inhibitor, a haloalkane inhibitor, a benzylcytosine inhibitor, and a benzylguanine inhibitor can be prepared similarly according to the method discussed above for the multi-functional linker having the structure of Formula 17.

### Method of Preparing a Megamolecule

Another aspects of the disclosure provides a method for preparing a megamolecule, the method comprising: reacting a multi-functional linker according to any of claims 12-51 with a first bio-matter and a second bio-matter to form the megamolecule, wherein the first bio-matter has a first active enzyme including a first terminus, and the second bio-matter has a second active enzyme including a second terminus, wherein the multi-functional linker comprises a central compound, a first synthetic inhibitor and a second synthetic inhibitor each of which is coupled to the central compound, wherein the first synthetic inhibitor comprises a first functional group specific for irreversibly inhibiting the first reactive enzyme at the first terminus, wherein the second synthetic inhibitor comprises a second functional group specific for irreversibly inhibiting the second reactive enzyme at the second terminus, wherein the reacting comprises irreversibly inhibiting the first active enzyme at the first terminus by the first synthetic inhibitor to form a covalent bond between the first functional group and the first terminus, and thus coupling the first bio-matter to the first synthetic inhibitor, wherein the reacting comprises irreversibly inhibiting the second active enzyme at the second terminus by the second synthetic inhibitor to form a covalent bond between the second functional group and the second terminus, and thus coupling the second bio-matter to the second synthetic inhibitor, wherein the first and second functional groups are each independently selected from the group of a fluorosulfate (F—SO₂—O—), an arylfluorosulfate (F—SO₂—O—Ar), a sulfonyl fluoride (F—SO₂—), an arylsulfonyl fluoride (F—SO₂—Ar), ap-nitrophenyl phosphonate (pNPP), a chloro-pyrimidine (CP), a haloalkane, a benzylcytosine and a benzylguanine group, and wherein the megamolecule has a molecular weight in a range of about 1.0 KDa to about 10.0 MDa.

In embodiments, the first and second active enzymes are each independently selected from the group of CrabTag, cutinase, SnapTag, HaloTag and ClipTag. In embodiments, the first synthetic inhibitor is the synthetic inhibitor of the disclosure. In embodiments, the first synthetic inhibitor is a synthetic inhibitor having a structure of Formula 10. In embodiments, the multi-functional linker is the multi-functional linker of the disclosure.

In embodiments, the method further comprises preparing the first synthetic inhibitor according to the method of the disclosure discussed herein above.

In embodiments, the method further comprises preparing the multi-functional linker according to the method of the disclosure discussed herein above.

In embodiments, the megamolecule maintains at least 95 wt. % intact after storage at room temperature in PBS solution at a concentration of about 1 μM for up to about 2 weeks.

In embodiments, the first terminus of the first active enzyme comprises a first active site having a tyrosine (tyr) residue. In embodiments, the first terminus of the first active enzyme comprises a arginine 112 (Arg112) residue and a arginine 133 (Arg133) residue each of which is within 5 angstroms of distance to the tyrosine (tyr) residue at the first terminus of the first reactive enzyme. In embodiments, the first active enzyme is a human-derived cellular retinoic acid binding protein II (CRABP2) (CrabTag), and the first synthetic inhibitor is the synthetic inhibitor having the structure of Formula 10. In embodiments, the second active enzyme is selected from the group of CrabTag, cutinase, SnapTag, HaloTag and ClipTag. In embodiments, the second synthetic inhibitor is selected from the group of a synthetic inhibitor has a structure according to Formula 10, a p-nitrophenyl phosphonate (pNPP) inhibitor, a chloro-pyrimidine (CP) inhibitor, a haloalkane inhibitor, a benzylcytosine inhibitor, and a benzylguanine inhibitor.

In embodiments, wherein the reacting further comprises reacting the multi-functional linker with a third bio-matter having a third active enzyme which has a third terminus, wherein the multi-functional linker further comprises a third synthetic inhibitor coupled to the central compound, the third synthetic inhibitor has a third functional group specific for irreversibly inhibiting the third active enzyme at the third terminus, wherein the reacting comprises irreversibly inhibiting the third active enzyme by the third synthetic inhibitor to form a covalent bond between the third functional group and the third terminus, and thus coupling the third bio-matter to the third synthetic inhibitor, and wherein the third functional group is selected from the group of a fluorosulfate (F—SO₂—O—), an arylfluorosulfate (F—SO₂—O—Ar), a sulfonyl fluoride (F—SO₂—), an arylsulfonyl fluoride (F—SO₂—Ar), a p-nitrophenyl phosphonate (pNPP), a chloro-pyrimidine (CP), a haloalkane, a benzylcytosine and a benzylguanine group.

In embodiments, the third active enzyme is selected from the group of CrabTag, cutinase, SnapTag, HaloTag and ClipTag. In embodiments, the third synthetic inhibitor is selected from the group of the synthetic inhibitor having the structure of Formula 10, p-nitrophenyl phosphonate (pNPP) inhibitor, chloro-pyrimidine (CP) inhibitor, haloalkane inhibitor, benzylcytosine inhibitor, and benzylguanine inhibitor. In embodiments, the second active enzyme is cutinase, and the second synthetic inhibitor is p-nitrophenyl phosphonate (pNPP) inhibitor. In embodiments, the third active enzyme is SnapTag, and third synthetic inhibitor is the chloro-pyrimidine (CP) inhibitor or the benzylguanine inhibitor.

In embodiments, the central compound has a chemical structure according to Formula 20.

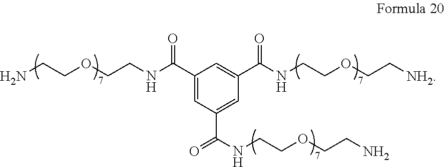

In embodiments, the multi-functional linker has a structure according to Formula 17 which comprises: the central compound having the structure of Formula 20, the synthetic inhibitor having the structure of Formula 10, the p-nitrophenyl phosphonate (pNPP) inhibitor, and the chloro-pyrimidine (CP) inhibitor, each of the inhibitors is coupled to the central compound.

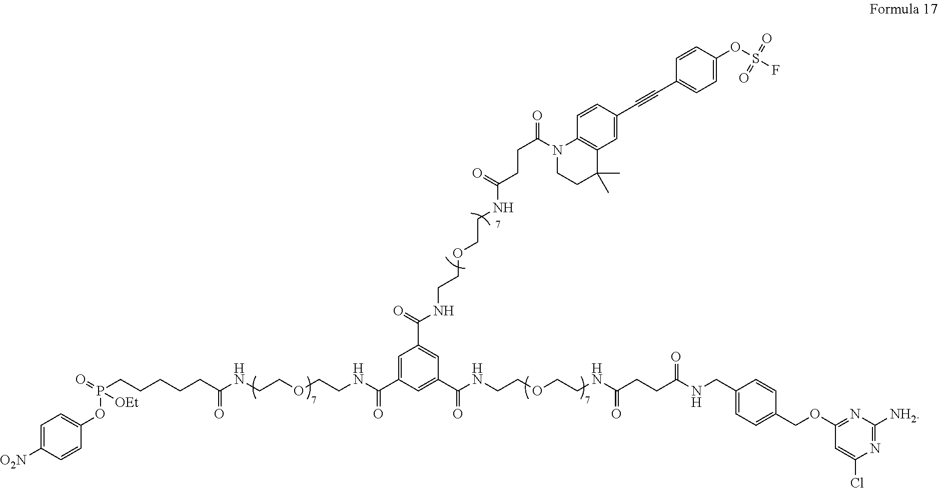

In embodiments, the megamolecule comprises: the multi-functional linker having the structure according to Formula 17; the CRABP2 (CrabTag) covalently attached to the multi-functional linker through a covalent bond formed between the synthetic retinoid inhibitor having the structure of Formula 10 and the CrabTag; the cutinase covalently attached to the multi-functional linker through a covalent bond formed between the p-nitrophenyl phosphonate (pNPP) inhibitor and the cutinase; and the SnapTag covalently attached to the multi-functional linker through a covalent bond formed between the chloro-pyrimidine (CP) inhibitor and the SnapTag. In embodiments, the megamolecule has a molecule weight of 62,232 Da.

In embodiments, the central compound further comprises additional 1-4 synthetic inhibitors each of which is coupled to the central compound, wherein each of the additional synthetic inhibitors is specific for irreversibly inhibiting an active enzyme of an additional bio-matter having the active enzyme.

In embodiments, wherein the first, second and third bio-matters are each independently selected from the group of a protein, an enzyme, an antibody fragment, a peptide, and a fusion protein.

In embodiments, one or more of the first, second or third bio-matters are fusion proteins. In embodiments, the megamolecule comprises two or more of the multi-functional linkers, and one or more of the bio-matters are fusion proteins.

In embodiments, the megamolecule comprises: two multi-functional linkers having the structure according to Formula 17; two CRABP2 (CrabTag) each coupled to the synthetic inhibitor having the structure of Formula 10 of each of the two multi-functional linkers, two cutinase coupled to one of the p-nitrophenyl phosphonate (pNPP) inhibitors of each of the two multi-functional linkers; and a fusion protein comprising SnapTag-SnapTag having two ends, each end coupled to one of the chloro-pyrimidine (CP) inhibitors of each of the two multi-functional linkers. In embodiments, the megamolecule has a molecular weight of 124.5 KDa.

In embodiments, the reacting comprises reacting the multi-functional linker with the first and second bio-matters in one pot. In embodiments, the reacting comprises reacting the multi-functional linker with the first, second and third bio-matters in one pot. In embodiments, the reacting comprises reacting the one or more multi-functional linker with the one or more bio-matters in one pot. In embodiments, the reacting does not include any intermediate protecting and deprotecting reactions.

Other megamolecules having the same central compound having the structure of Formula 20 with different synthetic inhibitor-enzyme pair can be prepared similarly according to the method discussed above for the megamolecule having the structure of Configuration 18.

The methods of preparing non-limiting examples of the synthetic inhibitors, the multi-functional linkers, and the megamolecule assemblies are detailed below.

### Synthesis of the CRABP2 Synthetic Retinoid Covalent Inhibitor

Chen et al. reported covalent inhibition of CRABP2, at an active site tyrosine residue, by an arylfluorosulfate electrophile. This sulfur fluoride exchange (SuFEx) click reaction was activated by two proximal arginine residues (Arg112 and Arg133) that electrostatically stabilized the reactive sulfur fluoride bond through the formation of a sulfate ester, lowering the pKₐ of the nucleophile and accelerating the inhibition reaction. The rate of reaction with this inhibitor, however, was not well-suited for megamolecule assembly.

Christie at al. and Chrisholm et al. each reported synthetic retinoids having a highly-conjugated, linear backbone. This class of synthetic retinoids have high affinity for CRABP2 owes to their highly-conjugated, linear backbone for binding into the hydrophobic ligand binding pocket of the protein. However, the reported method resulted in a non-covalent CRABP2-synthetic retinoid complex, and the reported class of synthetic retinoids further have relatively poor specific binding selectivity to the CRABP2.

The present disclosure provides a design of the covalent inhibitor for covalent inhibition of CRABP2, at an active site tyrosine (tyr) residue, by an arylfluorosulfate electrophile. This sulfur fluoride exchange (SuFEx) click reaction is activated by two proximal arginine residues (Arg112 and Arg133) that electrostatically stabilized the reactive sulfur fluoride bond through the formation of a sulfate ester, lowering the pKa of the nucleophile and accelerating the inhibition reaction. The covalent inhibitor of the present disclosure is further designed to incorporate a highly-conjugated, linear backbone for binding into the hydrophobic ligand binding pocket of the protein.

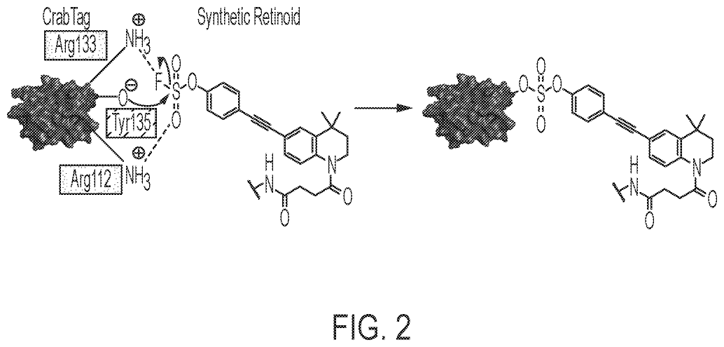

The present disclosure provides a synthetic inhibitor or synthetic retinoid with a modification at the hydrophobic region for ligation onto the linkers. The polar region of the retinoid displays the necessary arylfluorosulfate group for covalent modification by SuFEx with the monomeric CRABP2 active site tyrosine nucleophile, as shown in FIG. 2.

The present disclosure provides a method to prepare the synthetic inhibitor having a structure according to Formula 10 in a 10-step convergent synthesis through the conjugation of π-donor having a structure of Formula 4 and π-acceptor having a structure of Formula 7, as detailed in Scheme 1. The former is synthesized from 4-iodoaniline by acylation and Friedel-Crafts cyclization followed by reduction with borane dimethyl sulfide complex to yield tetrahydroquinoline having a structure of Formula 3. Acylation with 4-chloro-4-oxobutyrate gives para-iodoaniline derivative having a structure of Formula 4. The π-acceptor partner having a structure of Formula 7 is synthesized following an approach reported by Freccero et al., 4-Iodophenol is protected by O-acetylation (having a structure of Formula 5), coupled to trimethylsilylacetylene by Sonogashira cross-coupling to form a compound having a structure of Formula 6, and desilylated with potassium fluoride. The protected synthetic retinoid having a structure of Formula 8 is then conjugated through Sonogashira coupling of π-acceptor (having a structure of Formula 7) and π-donor (having a structure of Formula 4), followed by saponification to reveal non-covalent retinoid inhibitor having a structure of Formula 9 with a succinic acid handle for chemical ligation. The method further comprises sulfonylation of the terminal phenol of the non-covalent retinoid inhibitor having the structure of Formula 9 with [4-(acetylamino)phenyl]imidodisulfuryl difluoride (AISF) to give the synthetic inhibitor or retinoid having a structure of Formula 10.

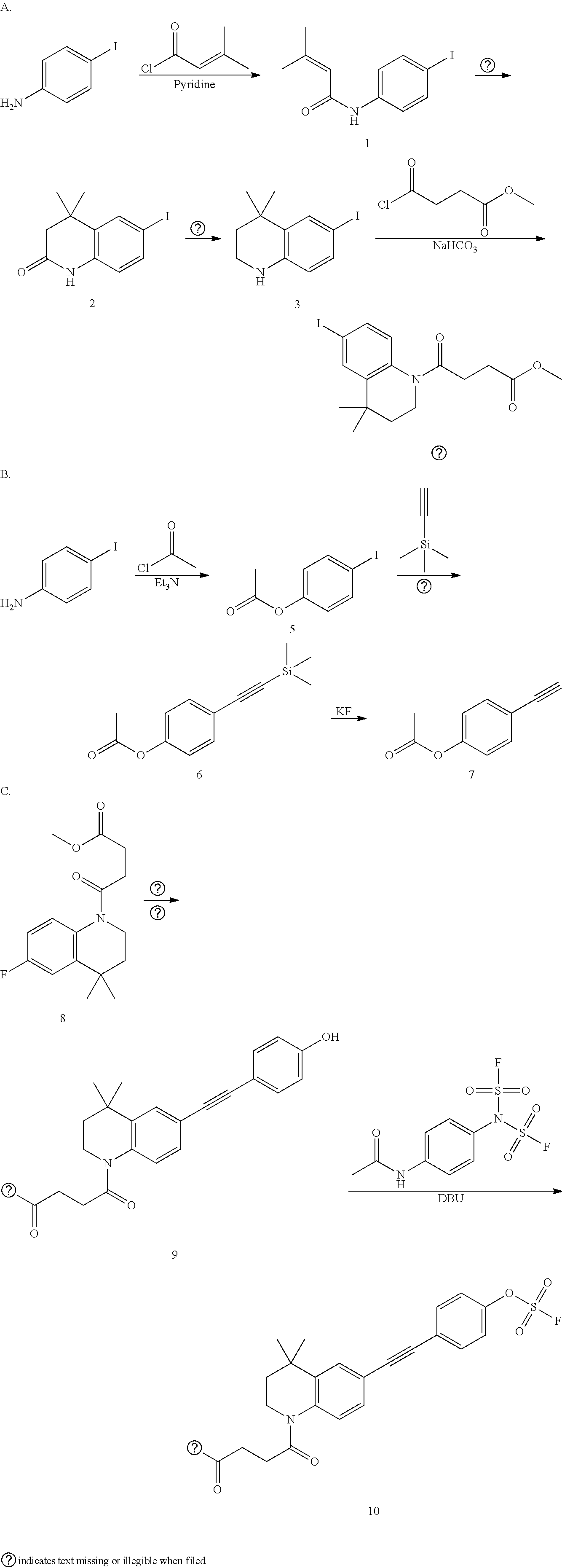

In embodiments, the method of preparing the synthetic retinoid inhibitor 4-(6-((4-((fluorosulfonyl)oxy)phenyl)ethynyl)-4,4-dimiethyl-3,4-dihydroquinolin-1(2H)-yl)-4-oxobutanoic acid having the chemical structure according to Formula 10 comprises:
a) synthesizing N-(4-Iodophenyl)-3-methyl-2-butenamide, 1 by admixing and reacting 4-iodoaniline and 3,3-dimethylacryloyl chloride by acylation;
b) synthesizing 6-iodo-4,4-dimethyl-3,4-dihydroquinolin-2(1H)-one, 2 through Friedel-Crafts cyclization of 1;
c) reducing 2 with borane-dimethyl sulfide complex to yield tetrahydroquinoline 3, wherein the reaction time is in a range of about 8-12 hours which is critical to achieve the desired yield;
d) synthesizing n-donor para-iodoaniline derivative 4 by acylation of 3 with methyl 4-chloro-4-oxobutyrate;
e) admixing and reacting 4-Iodophenol with acetyl chloride to yield 4-iodophenyl acetate, 5;
f) coupling 5 to trimethylsilylacetylene by Sonogashira cross-coupling to yield 4-((trimethylsilyl)ethynyl)phenyl acetate, 6;
g) desilylating 6 with potassium fluoride to yield 4-ethynylphenyl acetate, 7;
h) synthesizing Methyl 4-(6-((4-(acetyloxy)phenyl)ethynyl)-4,4-dimethyl-3,4-dihydroquinolin-1(2H)-yl)-4-oxobutanoate, 8 (protected synthetic retinoid 8) through Sonogashira coupling of z-acceptor 7 and π-donor 4;
i) synthesizing 4-(6-((4-hydroxyphenyl)ethynyl)-4,4-dimethyl-3,4-dihydroquinolin-1(2H)-yl)-4-oxobutanoic acid, 9 through saponification of 8 to reveal non-covalent retinoid inhibitor 9 with a succinic acid handle for chemical ligation; and
j) synthesizing 4-(6-((4-((fluorosulfonyl)oxy)phenyl)ethynyl)-4,4-dimethyl-3,4-dihydroquinolin-1(2H)-yl)-4-oxobutanoic acid, 10 through sulfonylation of the terminal phenol of 9 with [4-(acetylamino)phenyl]imidodisulfuryl difluoride (AISF) to give synthetic retinoid 10, wherein the reaction time is about 1 hour which is critical to achieve the desired yield.

In embodiments, the method of preparing the synthetic retinoid inhibitor having a structure according to Formula 10, comprises:
a) synthesizing N-(4-Iodophenyl)-3-methyl-2-butenamide, 1, comprising: admixing and reacting 4-iodoaniline and 3,3-dimethylacryloyl chloride in in a DCM and pyridine solvent system at room temperature for acylation for about 1-50, about 5-30, about 6-20, about 8-18, about 10-16, or about 14 hours to form the product 1;

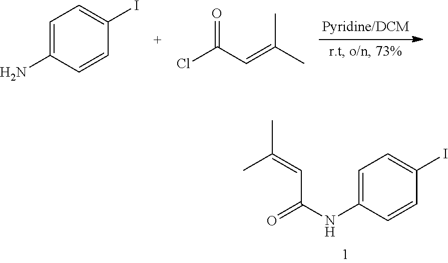

b) synthesizing 6-iodo-4,4-dimethyl-3,4-dihydroquinolin-2(1H)-one, 2, comprising: admixing product 1 and AlCl₃ under nitrogen to form a mixture, dissolving the mixture in DCM solvent, and reacting at room temperature for about 1-20, about 1-10, about 1-5, about 2-3, or about 2.5 hours to for the product 2 by Friedel-Crafts cyclization of 1;

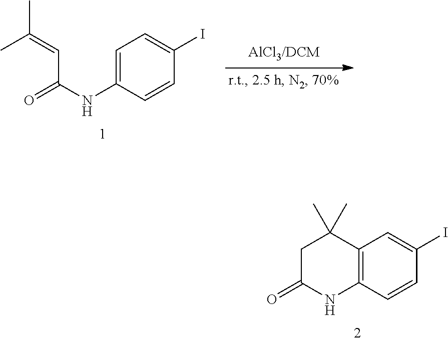

c) synthesizing 6-iodo-4,4-dimethyl-1,2,3,4-tetrahydroquinoline, 3, comprising: admixing product 2 and toluene under nitrogen to form a mixture, adding borane dimethyl sulfide complex at 2M in THF to the mixture, reacting at a reflux for about 7-13, about 8-12, or about 8 hours to reduce product 2 with borane dimethyl sulfide complex to yield 3 (6-iodo-4,4-dimethyl-1,2,3,4-tetrahydroquinoline), wherein the reaction time at the reflux is critical in achieving high yield and reacting shorter than 8 hours and longer than 12 hours resulted in a loss if yield;

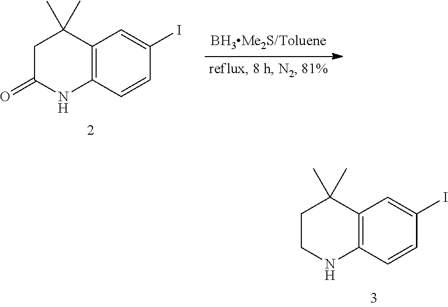

d) synthesizing Methyl 4-(6-iodo-4,4-dimethyl-3,4-dihydroquinolin-1(2H)-yl)-4-oxobutanoate, 4 (π-donor para-iodoaniline derivative), comprising: adding product 3 to DCM to form a solution, adding NaHCO₃ to the solution and stirring for about 1-120, about 5-80, about 10-60, about 15-50, about 20-40, about 25-35, or about 30 minutes, adding methyl 4-chloro-4-oxobutyrate dropwise, reacting at room temperature for about 1-50, about 5-30, about 6-20, about 8-18, about 10-16, or about 14 hours for acylation of 3 with methyl 4-chloro-4-oxobutyrate to form product 4;

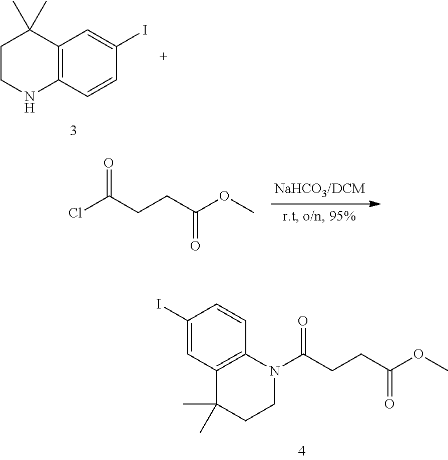

e) synthesizing 4-iodophenylacetate, 5, comprising one or more steps of: admixing 4-Iodophenol with DCM in a container on ice, adding Et₃N to the container and stirring for about 5-60, about 10-50, about 20-40, about 25-35, or about 30 minutes to form a mixture, adding acetyl chloride to the mixture, raising the temperature to room temperature, and reacting 4-Iodophenol with acetyl chloride while stirring for about 0.1-10, about 1-5, about 1-3, or about 2 hours to yield 4-iodophenyl acetate, 5;

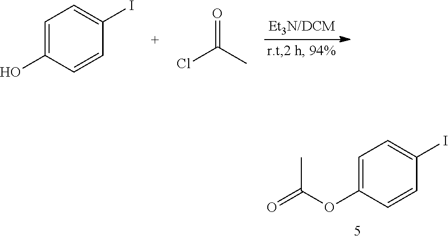

f) synthesizing 4-((trimethylsilyl)ethynyl)phenyl acetate, 6, comprising: admixing product 5, Pd(PPh3)2Cl2, and CuI in a container; purging the container with N2; adding DMF to the container; raising the temperature to 50 C; adding trimethylsilyl-acetylene and Et3N to the container; reacting for about 1-10, about 1-5, about 1-4, about 2.5 hours to couple product 5 to trimethylsilylacetylene by Sonogashira cross-coupling to yield 4-((trimethylsilyl)ethynyl)phenyl acetate, 6;

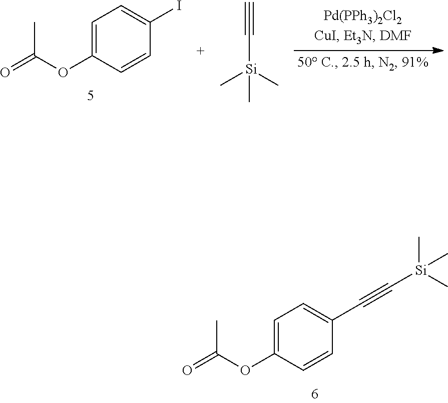

g) synthesizing 4-ethynylphenyl acetate, 7, comprising: admixing product 6, DMF and H₂O to form a mixture; adding potassium fluoride to the mixture; and reacting while stirring for about 0.1-10, about 0.5-5, about 0.6-3, about 0.7-2, about 0.8-1.5, about 0.9-1.1, or about 1 hour at room temperature to desilylate 6 with potassium fluoride to yield 4-ethynylphenyl acetate, 7;

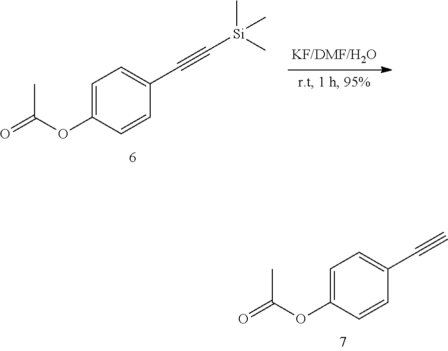 [SMILES: CC(=O)Oc1ccc(C#C[Si](C)(C)C)cc1 ; C#Cc1ccc(OC(C)=O)cc1]

h) synthesizing Methyl 4-(6-((4-(acetyloxy)phenyl)ethynyl)-4,4-dimethyl-3,4-dihydroquinolin-1(2H)-yl)-4-oxobutanoate, 8 (protected synthetic retinoid 8) through Sonogashira coupling of z-acceptor 7 and π-donor 4, comprising: admixing product 4, Pd(PPh3)2Cl2 and CuI to a container; purging the container with N₂; raising the temperature to 50° C.; adding DMF while stirring; adding product 7 and Et3N while stirring; reacting for about 1-10, about 1-5, about 2-3, or about 2.5 hours to form product 8;

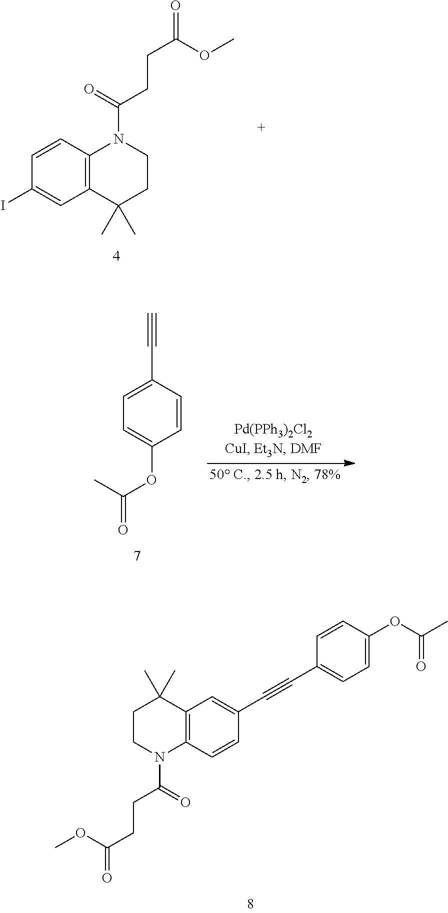 [SMILES: COC(=O)CCC(=O)N1CCC(C)(C)c2cc(I)ccc21 ; C#Cc1ccc(OC(C)=O)cc1 ; COC(=O)CCC(=O)N1CCC(C)(C)c2cc(C#Cc3ccc(OC(C)=O)cc3)ccc21]

i) synthesizing 4-(6-((4-hydroxyphenyl)ethynyl)-4,4-dimethyl-3,4-dihydroquinolin-1(2H)-yl)-4-oxobutanoic acid, 9 through saponification of 8 to reveal non-covalent retinoid inhibitor 9 with a succinic acid handle for chemical ligation, comprising: admixing product 8, MeOH and NaOH by stirring at room temperature for about 0.1-5, about 0.5-2, or about 1 hour; adding HCl and EtOAc; and washing and purifying to form product 9; and

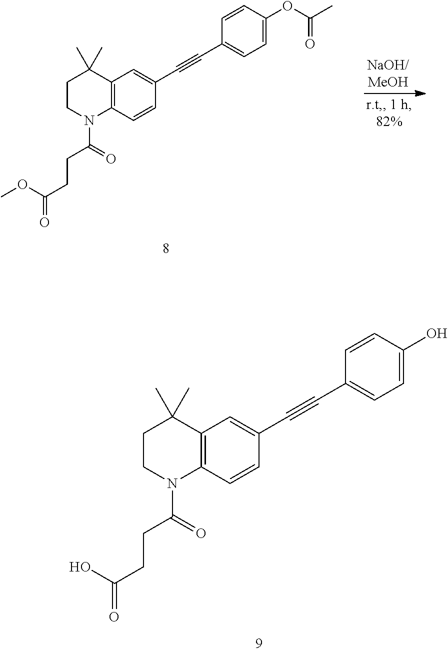 [SMILES: COC(=O)CCC(=O)N1CCC(C)(C)c2cc(C#Cc3ccc(OC(C)=O)cc3)ccc21 ; CC1(C)CCN(C(=O)CCC(=O)O)c2ccc(C#Cc3ccc(O)cc3)cc21]

j) synthesizing 4-(6-((4-((fluorosulfonyl)oxy)phenyl)ethynyl)-4,4-dimethyl-3,4-dihydroquinolin-1(2H)-yl)-4-oxobutanoic acid, 10 through sulfonylation of the terminal phenol of 9 with [4-(acetylamino)phenyl]imidodisulfuryl difluoride (AISF) to give synthetic retinoid 10, comprising: dissolving product 9 in THF to form a mixture; adding [4-(acetylamino)phenyl]imidodisulfuryl difluoride (AISF); adding DBU dropwise; reacting while stirring at room temperature for about 50-70, about 55-65, about 58-62, or about 60 minutes to form the synthetic retinoid 10.

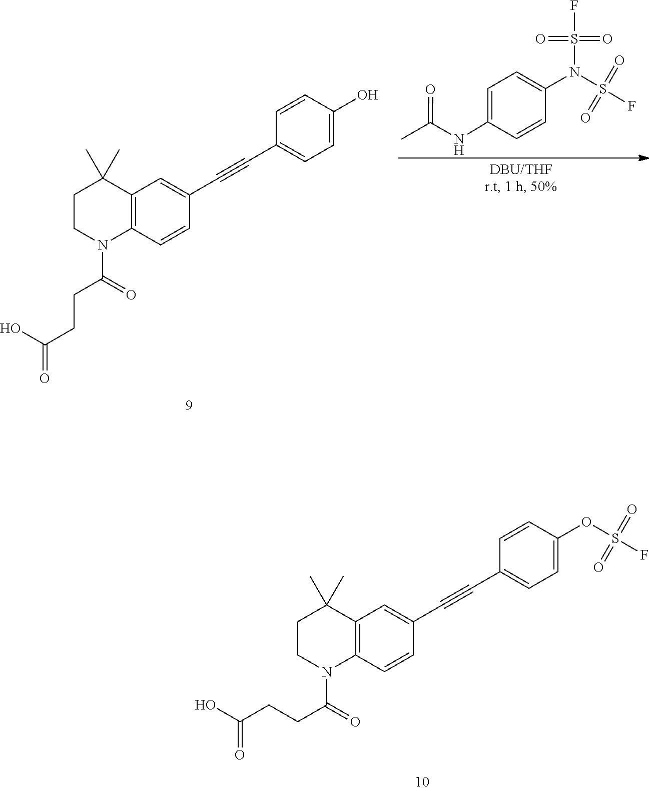 [SMILES: CC1(C)CCN(C(=O)CCC(=O)O)c2ccc(C#Cc3ccc(O)cc3)cc21 ; CC(=O)Nc1ccc(N(S(=O)(=O)F)S(=O)(=O)F)cc1 ; CC1(C)CCN(C(=O)CCC(=O)O)c2ccc(C#Cc3ccc(OS(=O)(=O)F)cc3)cc21]

In embodiments, step j) of the method above comprises: dissolving 1.9165 g (5.083 mmol, 1 eq.) of product 9 in 25.41 mL of THF to form a mixture; adding 1.713 g of [4-(acetylamino)phenyl]imidodisulfuryl difluoride (AISF); adding 1.681 mL of DBU dropwise over a 30 second period; reacting while stirring at room temperature for about 50-70, about 55-65, about 58-62, or about 60 minutes to form the synthetic retinoid 10 mixture. In embodiments, step j) of the method further comprises: at the completion of the reaction, diluting the synthetic retinoid 10 mixture at 10:1 dilution in EtOAc; collecting the organic layer and washing the organic layer 3× with 0.5 M HCl (aq.), 2× with sat. HNaCO3 (aq.), and finally acidifying by washing 3× with 0.5 M HCl (aq.); further washing the organic layer with brine 3×; drying with Na2SO4; filtering; concentrating to form a crude mixture; purifying the crude mixture by silica chromatography (10:90 MeOH:DCM); and then concentrating using the rotovap to form the final product, 10 (1.16 g, 50%), which is recovered as a white-pink solid.

The inventors found that this step j) of the method is the most complex synthesis step, as seen by the lowest yield. The inventors have tried many combinations of the reaction time, conditions, selection of different combination of solvents for washing, order of washing by the different solvent, selection of purification methods and column, and drying and concentrating methods and conditions. The inventors surprisingly found that the reaction time of about 1 hour is critical in achieving high yield. Any reaction time longer than 1 hour resulted in a loss of yield. Any reaction time less than 1 hour resulted in little yield. Further, the washing and purification process involves several acidic and basic work ups. Many products exist, so getting the right product out by work up and column was challenging. The inventors further surprisingly found that the order of work up (washing and purification) made it highly specific, and is critical in achieving the right final product and high yield.

In embodiments, the step a) further comprises one or more of the following steps: adding 4-iodoaniline to solvent DCM to prepare a 4-iodoaniline solution having a concentration in a range of 0.001-1 g/ml, 0.01-0.5, 0.02-0.4, 0.03-0.3, 0.03-0.2, 0.04-0.1, 0.05-0.09, 0.06-0.08, 0.06-0.07, or 0.0625; adding pyridine to the solution at room temperature to form a mixture; stirring the mixture for about 30 minutes, the volume ratio of pyridine:DCM is in a range of 0.001:1-1:1, 0.01:1-0.05:1, 0.02:1-0.04:1, or 0.02:1-0.03:1, 0.024:1-0.025:1, or 0.24:1 vol/vol; putting the mixture on ice and adding 3,3-dimethylacryloyl chloride dropwise to the mixture, the ratio weight ratio of 4-iodoaniline:3,3-dimethylacryloyl chloride is in a range of about 10:1-1:10, about 5:1-1:5, about 3:1-1:3, about 3:1-1:2, about 2:1-1:1, or about 1.76:1; returning the mixture to room temperature and stirring for about 1-24, about 6-20, about 8-18, about 10-16, about 10-14, or about 12 hours; diluting the reacted mixture with water and extracting the formed N-(4-Iodophenyl)-3-methyl-2-butenamide (Product 1) about 1-10 or about 3 times; washing with 1 M HCl (aq.), sat. NaHCO₃(aq.), and then with brine; drying with Na2SO4, filtrating, and concentrating; and recrystallizing in EtOH to obtain pure product 1 (white powder).

In embodiments, the step c) of the method may further comprises one or more of the following steps: adding 6-iodo-4,4-dimethyl-3,4-dihydroquinolin-2(1H)-one (product 2) (5.23 g, 1 eq.) to 39.2 mL toluene to a container such as a flask to form a mixture; evacuating the flask with N₂ three times; adding 10.974 mL of borane dimethyl sulfide complex at 2M in THF to the mixture; reacting at reflux for about 1-20, about 5-16, about 6-14, about 8-12, or about 8 hours; keeping stirring for 30 min at room temperature; adding 33 mL of 10% Na2CO3 (aq.); extracting with EtOAc; washing once with H2O, and washing 3 times with brine; drying the organic layer with Na2SO4, filtering, and concentrating to yield the crude mixture; purifying the crude mixture using silica chromatography (10:90 EtOAc:Hexanes+1% Et3N); and concentrating to reveal a pure yellow oil product 3 (6-iodo-4,4-dimethyl-1,2,3,4-tetrahydroquinoline). The inventors surprising found that the refluxing time is critical to achieve a high yield. When the refluxing time is about 8 hours, the yield of the product 3 is about 81%. However, when the refluxing time is less than about 8 hours or higher than about 12 hours resulted a loss of yield.

In embodiments, the step c) of the method may further comprises one or more of the following steps: dissolving 1.9165 g (5.083 mmol, 1 eq.) of product 9 (4-(6-((4-hydroxyphenyl)ethynyl)-4,4-dimethyl-3,4-dihydroquinolin-1(2H)-yl)-4-oxobutanoic acid) in 25.41 mL of THE to form a mixture; adding 1.713 g of [4-(acetylamino)phenyl]imidodisulfuryl difluoride (AISF) to the mixture to form a reaction mixture; adding 1.681 mL of DBU dropwise to the reaction mixture over a 30 second period; reacting while stirring at room temperature for about 50-70 minutes, about 55-65 minutes, about 58-62 minute, about 1 hour, or 1 hour to yield a product mixture; diluting the product mixture EtOAc at a volume ratio of product mixture:EtOAc of about 20:1-1:1, about 15:1-5:1, or about 10:1; collecting the organic layer and washing 3 times with 0.5 M HCl (aq.), 2 times with sat. HNaCO3 (aq.), and washing 3 times with 0.5 M HCl (aq.) to acidify the organic layer; washing with brine 3 times; drying with Na2SO4, filtering, and concentrating to yield the crude mixture; purifying the crude mixture silica chromatography (10:90 MeOH:DCM); and concentrating using the rotovap to yield the final product 10 (1.16 g, 50%) as a white-pink solid. The yield of this step j) is about 50%.

The inventors found that this step j) of the method was the most complex, as seen by lowest yield. This was tried in many combinations. The inventors surprisingly found that the reacting while stirring time is critical in achieving a high final yield. The reaction is in a range of about 55-65, about 58-62, about 59-61 minutes, or about 60 minutes. The reaction was stirred at room temperature for one hour and achieved a final yield of 50%. When the reacting while stirring time was longer than about 65 minutes, it resulted in a loss of yield; and less than about 55 minutes reacting while stirring time resulted in little yield. The inventors further surprisingly found that the purification with several acidic and basic work ups is critical. Many products exist in the crude mixtures, so the inventors found that getting the right product out by work up and column was challenging, and the order of work up made it highly specific to obtain the right product. The inventors surprisingly found that the order of washing the organic layer to purify the product is critical to achieve a high yield of the final pure product 10, and the critical order of washing or purification steps are: first washing with 0.5 M HCl (aq.); second washing with sat. HNaCO3 (aq.); third washing with 0.5 M HCl (aq.); and fourth washing with brine. In embodiments, the inventors followed the critical order of washing or purification steps of first washing with 0.5 M HCl (aq.) for 3 times; second washing twice with sat. HNaCO3 (aq.); third washing 3 times with 0.5 M HCl (aq.); and fourth washing with brine 3 times and achieved a yield of 50% for the final pure product 10. After the washing and purification step, the obtained product was further dried with Na2SO4, filtered, and concentrated to yield the crude mixture. The crude mixture was further purified by silica chromatography (10:90 MeOH:DCM) and concentrated using the rotovap to yield the final pure product 10 as a white-pink solid.

### Engineering CRABP2

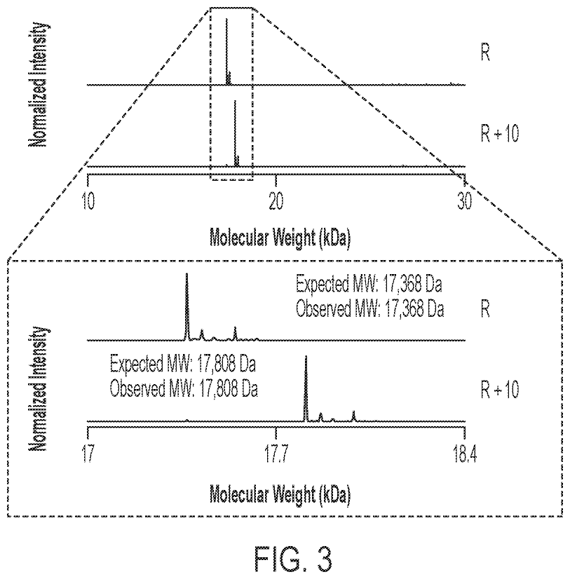

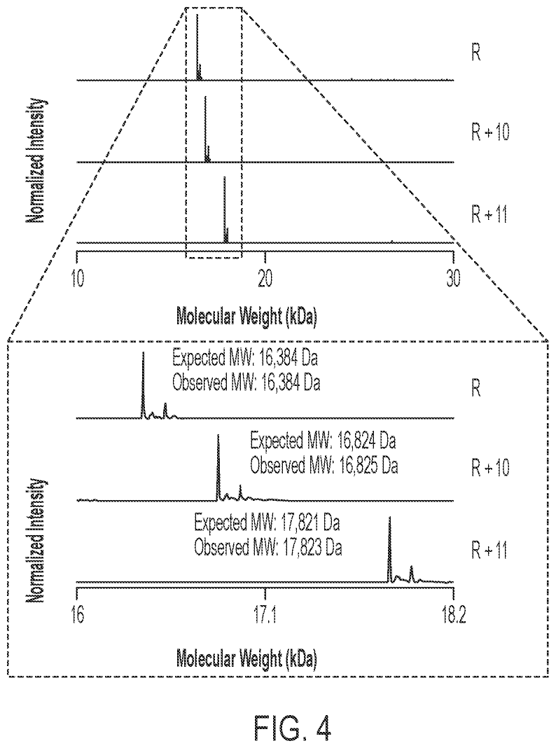

The present disclosure provides a method to construct a T7 expression plasmid incorporating the sequence for CRABP2 (referred to hereon as CrabTag) modified with a C-terminal His-tag. The method comprises transforming the plasmid into chemically competent E. coli and purifying the protein with immobilized metal affinity chromatography (IMAC). The method further comprises reacting the protein (10 μM) in phosphate buffered saline (PBS; 2.7 mM KCl, 138 mM NaCl, pH 7.4) with covalent inhibitor 10 (1.1 equivalents) and purifying the adduct using size exclusion chromatography (SEC). Electrospray ionization mass spectrometry (ESI-MS) has confirmed the presence of the adduct, as detailed in FIGS. 3 and 4.

Reacting CRABP2 Protein with Synthetic Retinoid Inhibitor 10 to Form CrabTag-10 Adduct and its Characteristics

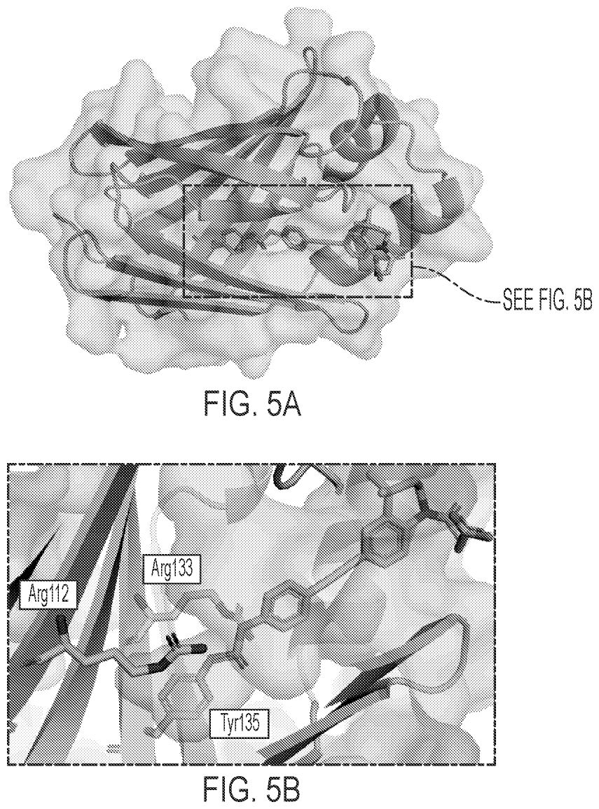

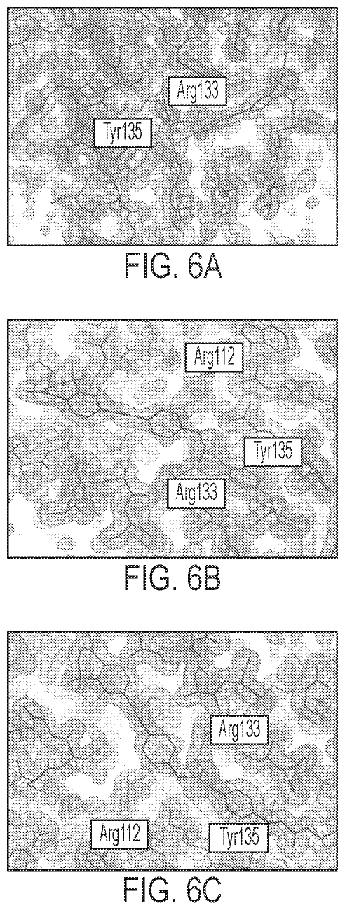

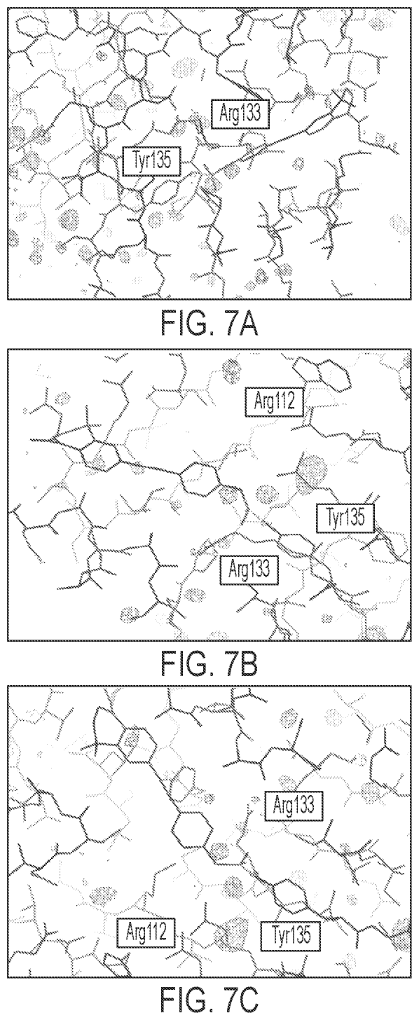

The present disclosure provides a method for growing crystals of the inhibited enzyme and obtaining an X-ray structure that diffracts to 1.8 Å (R_{work}=0.21, R_{free}=0.25), as shown in FIG. 5. The electron density map shows that the synthetic retinoid inhibitor forms a covalent sulfate ester adduct at the polar region of the inhibitor with the nucleophilic residue, Tyr135, as illustrated in FIGS. 6-9. A structural alignment comparison of the CRABP2 protein backbone in the CrabTag-10 adduct with the non-covalent CRABP2-synthetic retinoid complex from Chrisholm et al. (PDBID: 6HKR) shows high similarity (RSMD=1.18 Å over all atoms without refinement), as shown in Table 1 below. Consistent with the covalent CRABP2-diarylsulfate crystal structure from Chen et. Al., synthetic retinoid inhibitor 10 selectively modifies Tyr135 polar stabilization interactions between the aryl fluorosul fate electrophile. Further, the proximal hydrogen bonding donor (Arg112 and Arg133) side chains help to facilitate the covalent SuFEx modification of the Tyr135 residue.

TABLE 1
X-Ray data collection and refinement statistics.

| | Measurement |
|---|---|
| Crystallographic Data | |
| Wavelength | 0.979130 |
| Resolution range (Å) | 35.00-1.80 (1.851-1.804) |
| Space group | P 2 2₁2₁ |
| Unit Cell Dimensions (Å) | 36.604 69.655 120.845 |
| Unit Cell Angles | 90 90 90 |
| Total reflections | 183368 |
| Unique reflections | 29125 (2571) |
| Multiplicity | 6.5 (5.2) |
| Completeness (%) | 99.19 (89.91) |
| Mean I/sigma(I) | 42.1 (1.0) |
| R_{MERGE} | 0.146 (1.296) |
| R_{MEAS} | 0.160 (1.440) |
| R_{PIM} | 0.064 (0.617) |
| Crystallographic Refinement | |
| Reflections used in refinement | 29311 |
| Reflections used for R_{FREE} | 1493 |
| R_{WORK} | 0.206 (0.336) |
| R_{FREE} | 0.246 (0.316) |
| Number of non-hydrogen atoms | 5393 |
| RMS(bonds) | 0.010 |
| RMS(angles) | 1.69 |
| Ramachandran favored (%) | 99.5 |
| Ramachandran allowed (%) | 0.5 |
| Rotamer outliers (%) | 0.0 |
| Average B-factor (Å²) | 25.21 |
| macromolecules | 22.60 |
| ligands | 13.96 |
| solvent | 30.29 |

Values in parentheses correspond to the highest resolution shell.

### Synthesis of Synthetic Inhibitor Having a Structure of Formula 11

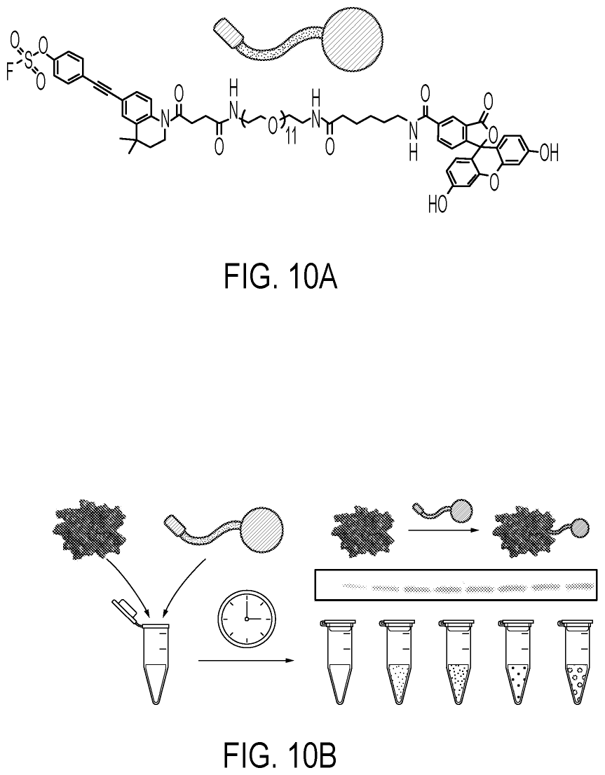

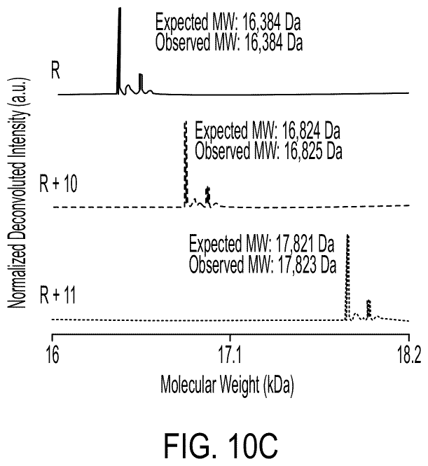

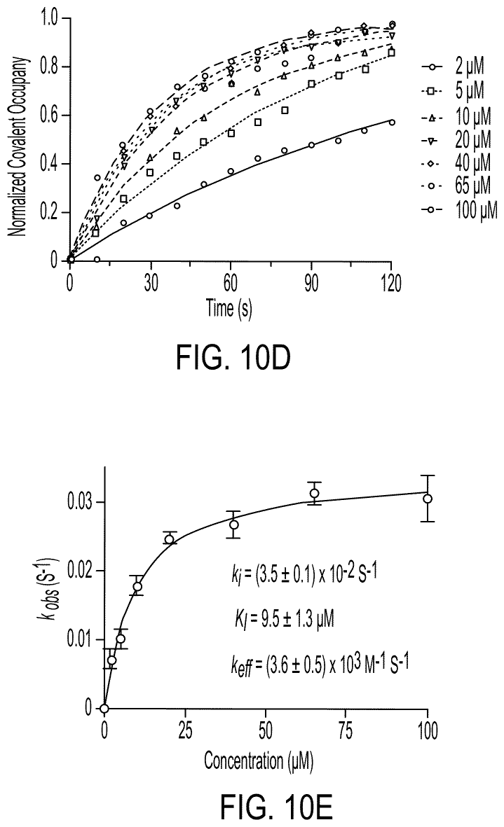

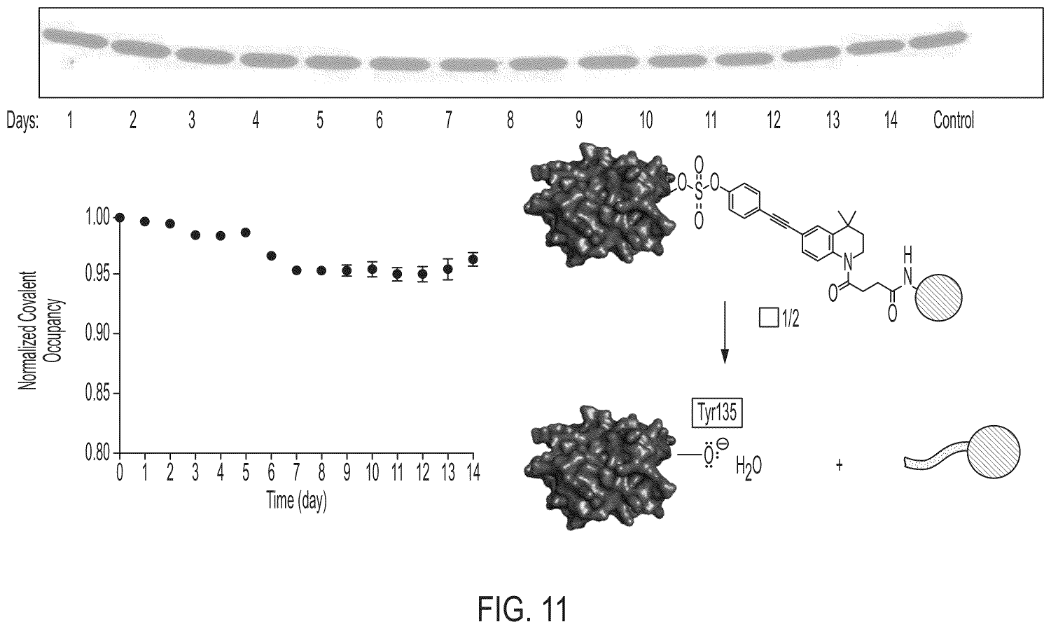

The present disclosure provides a method for synthesizing linker 11, which includes a fluorophore to characterize the kinetics for reaction of the covalent inhibitor and CrabTag, as shown in FIG. 10A. The inventors have run parallel reactions that varies the concentration of the linker (2, 5, 10, 20, 40, 65, and 100 μM) at a constant concentration of CrabTag (1 μM) and stops the reactions using Laemmli reducing buffer at times ranging from 0 to 120 seconds, as shown in FIG. 10B. Intact mass spectrometry analysis of the protein-inhibitor complexes for the reactions of CrabTag with either inhibitor 10 or 11 reveal monomeric adducts with only one modification that corresponds to the mass of either inhibitor, as shown in FIGS. 10C and 4. The inventors have separated the reaction products with SDS-PAGE and quantitated the mean fluorescence intensity of protein bands that corresponded to the adduct with ImageJ. The inventors have fit the kinetic data to a single-phase exponential decay to obtain observed rate constants, k_{obs}, as shown in FIG. 10D. The inventors have then plotted the k_{obs} values against the linker concentration to give a Michaelis-Menten curve, yielding the inactivation rate constant kᵢ and the equilibrium binding affinity of the enzyme-inhibitor complex K_{I}, as shown in FIG. 10E. The ratio of these parameters gives the second order rate constant for the covalent modification reaction, k_{eff}; as (3.6±0.5)×10³ M⁻¹s⁻¹. The inventors have also monitored the stability of the adduct by incubating the product (1 μM in PBS, 25° C.) and have found that over 95% of the protein domains in solution retain covalent occupancy of the linker after two weeks, as illustrated in FIG. 11.

### Assembling Megamolecules Using a Heterotrifunctional Linker Bearing Three Inhibitors

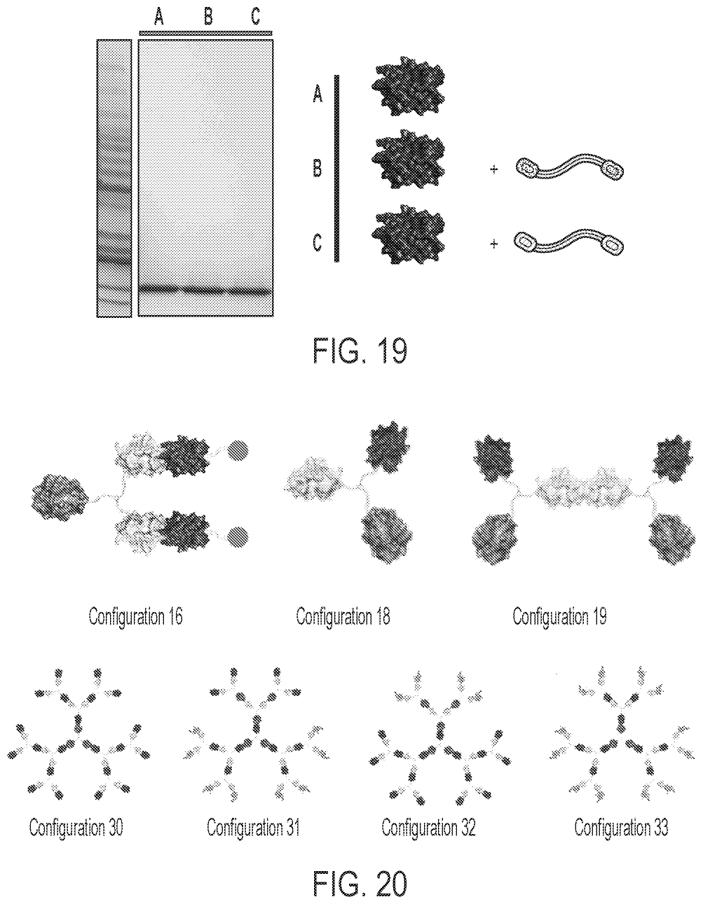

The inventors have further demonstrated a method for assembling megamolecules using three reactions—CrabTag with 10, cutinase with a pNPP inhibitor, and SnapTag with a CP inhibitor. Importantly, the inventors have first confirmed that each enzyme only specifically reacts with its paired covalent inhibitor as shown in FIG. 19 and therefore expected that branched megamolecule 16 could be assembled in a single, one-pot reaction from four reactants: cutinase, a SnapTag-CrabTag (SR) fusion protein, fluorescent linker 11, and a heterotrifunctional linker bearing two inhibitors for SnapTag and one inhibitor for cutinase.

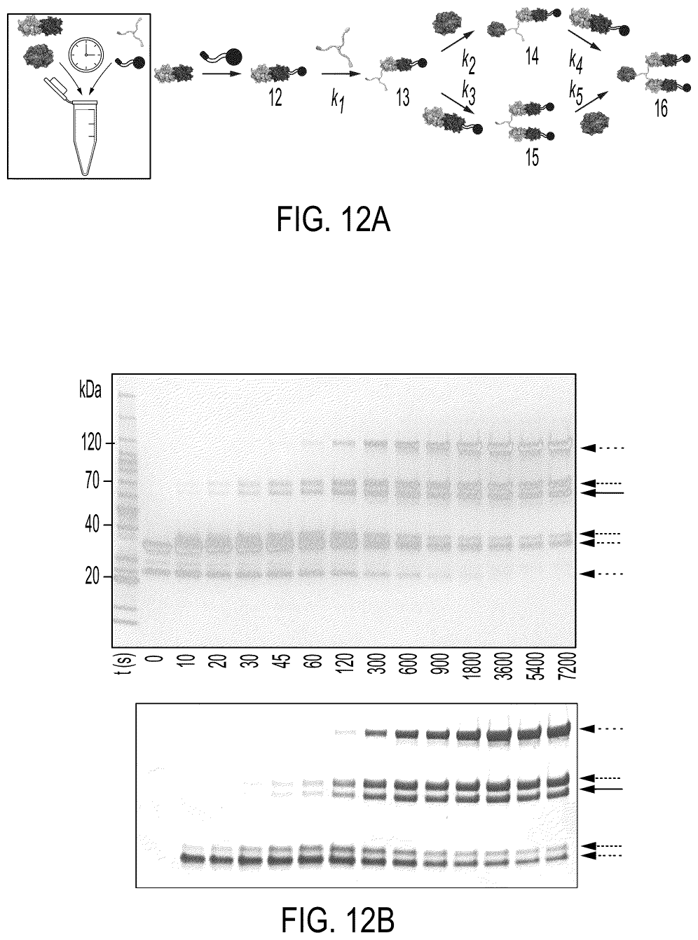

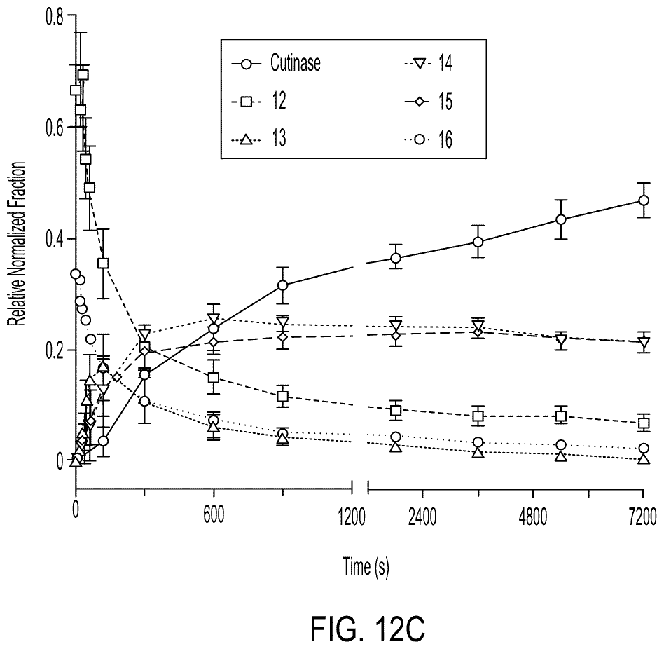

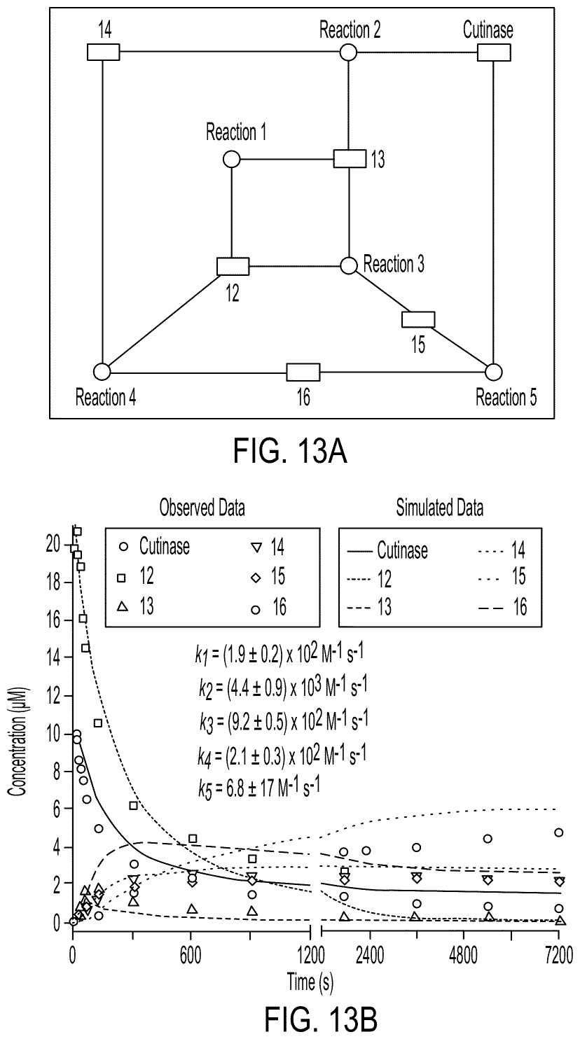

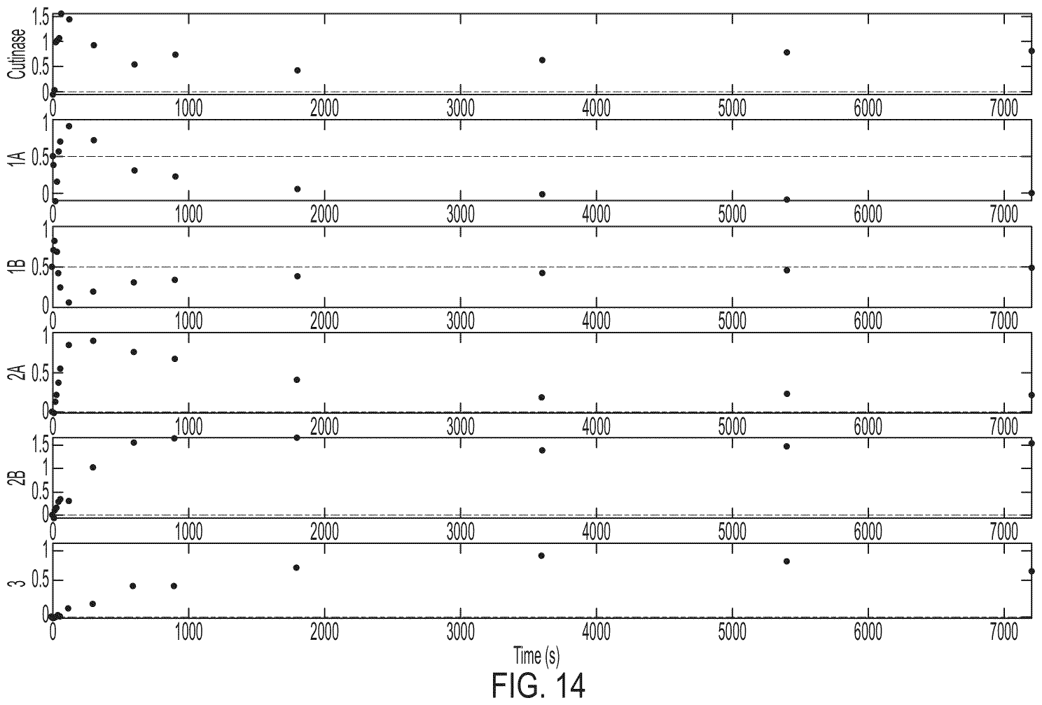

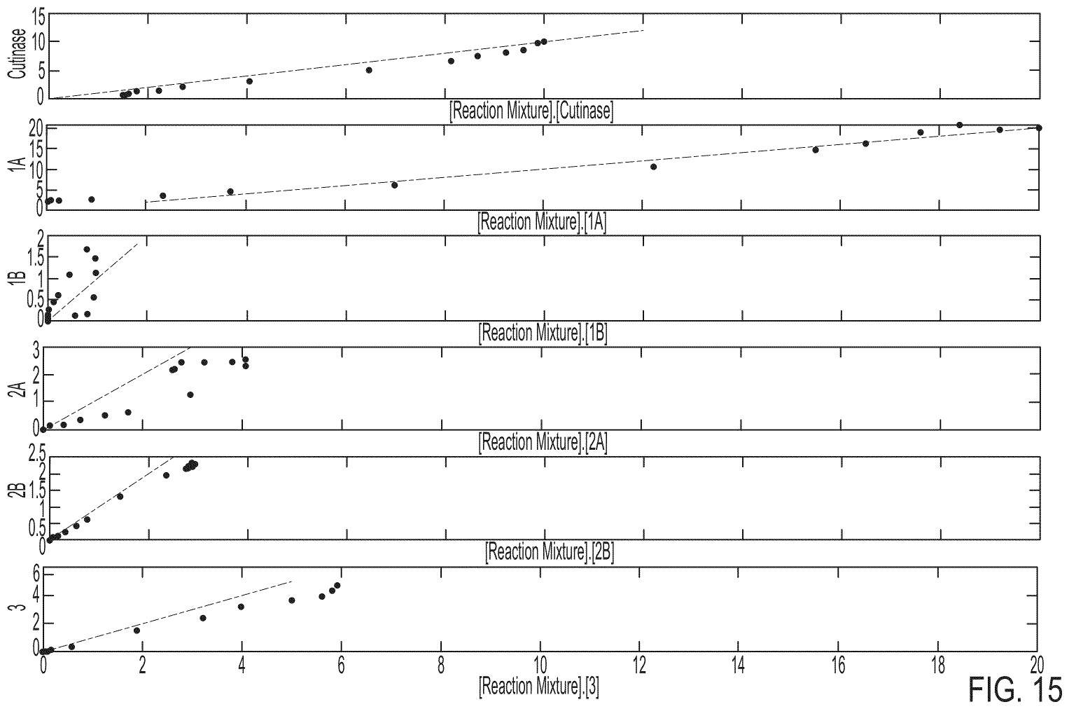

There are 120 discreet pathways that could give product 16. Consideration of the relative rates for the reactions of the three enzymes narrows the possible pathways to the two as shown in FIG. 12A. Because the reaction of CrabTag with its inhibitor is the fastest, the inventors expected that the SR fusion protein would first react with fluorescent linker 11 to give intermediate 12. Based on the relative rates for SnapTag and cutinase reactions with their inhibitors, the inventors have expected that intermediate 13 would form next. In the next step, the inventors have predicted that two reaction pathways—forming either intermediates 14 or 15—would compete to give final product 16. After performing the experiment, the inventors obtained time-resolved quantification of reaction intermediates and formation of final product 16 using both fluorescent and Coomassie stained SDS-PAGE, as illustrated in FIG. 12B. By monitoring the concentration of each intermediate, the inventors can assess the kinetics and determine the abundance of each reaction intermediate over time, as illustrated in FIG. 12C. The inventors have used the SimBiology application within MATLAB to develop a simple block diagram to model the batch reaction for the megamolecule assembly, as illustrated in FIG. 13. Due to the fast reaction kinetics of CrabTag inhibition, a key assumption to the present model is a pseudo-steady state hypothesis on the concentration of fluorescent linker 11. Using a non-linear regression as our statistical model, the inventors have simultaneously solved the system of differential rate law equations and estimated the second-order kinetic rate constant parameters from the model, as illustrated in FIGS. 13-15 and Tables 2-5. This experiment has revealed that a mechanistic approach could be used to describe and monitor the stepwise assembly of an atomically precise multi-protein scaffold over time.

TABLE 2
SimBiology Model and Simulation Unpooled Parameter Estimates.

| | Group | One Group |
|---|---|---|
| Reaction 1.k1 | Estimate | 0.000190084 |
| Reaction 1.k1 | Standard Error | 1.52449E−05 |
| Reaction 2.k2 | Estimate | 0.004363803 |
| Reaction 2.k2 | Standard Error | 0.000864685 |
| Reaction 3.k3 | Estimate | 0.00092345 |
| Reaction 3.k3 | Standard Error | 4.81802E−05 |
| Reaction 4.k4 | Estimate | 0.0002117 |
| Reaction 4.k4 | Standard Error | 2.82399E−05 |
| Reaction 5.k5 | Estimate | 6.82228E−06 |
| Reaction 5.k5 | Standard Error | 1.68514E−05 |

TABLE 3
SimBiology Model and Simulation Statistics.

| AIC | BIC | LogLikelihood | DFE | MSE | SSE |
|---|---|---|---|---|---|
| 233.8385799 | 245.9926639 | −111.9192899 | 79 | 0.894256063 | 70.64622894 |

TABLE 4
SimBiology Model and Simulation Residuals (Last Run).

| Time (S) | Cutinase | 12 | 13 | 14 | 15 | 16 |
|---|---|---|---|---|---|---|
| 0 | −0.06494 | 0.03247 | 0 | 0 | 0 | 0 |
| 10 | 0.032613418 | −0.453787758 | 0.421274548 | −0.015887247 | −0.02384135 | −0.020061657 |
| 20 | 0.984351008 | −2.437524394 | 0.634568486 | 0.237593282 | 0.044558854 | −0.016688331 |
| 30 | 1.013269297 | −1.397098107 | 0.370700393 | 0.424389991 | 0.063972652 | 0.006732175 |
| 45 | 1.065930178 | 0.256267382 | −0.159971699 | 0.735069701 | 0.11488876 | 0.042861764 |
| 60 | 1.553350977 | 0.801119822 | −0.510386238 | 1.08308373 | 0.132891661 | 0.023241089 |
| 120 | 1.438165085 | 1.622744227 | −0.884775777 | 1.700359264 | 0.125790778 | 0.225905764 |
| 300 | 0.927178624 | 0.881541104 | −0.63875616 | 1.804429498 | 0.404750203 | 0.355285751 |
| 600 | 0.544086633 | −0.759610212 | −0.398305005 | 1.517897303 | 0.620599945 | 0.83741883 |
| 900 | 0.740017392 | −1.094051198 | −0.327496198 | 1.349850468 | 0.661005626 | 0.823312906 |
| 1800 | 0.434688661 | −1.794140758 | −0.237067225 | 0.802498553 | 0.658447481 | 1.337135052 |
| 3600 | 0.631081695 | −2.094093125 | −0.14985521 | 0.367945589 | 0.554017209 | 1.673927482 |
| 5400 | 0.781770417 | −2.322820709 | −0.09733411 | 0.455916092 | 0.591465174 | 1.487330757 |
| 7200 | 0.810423764 | −2.032503886 | −0.03765147 | 0.432505099 | 0.612449043 | 1.237914904 |

TABLE 5
SimBiology Model and Simulation Covariance Matrix (Last Run).

| Parameters | log(Reaction 1.k1) | log(Reaction 2.k2) | log(Reaction 3.k3) | log(Reaction 4.k4) | log(Reaction 5.k5) |
|---|---|---|---|---|---|
| log(Reaction 1.k1) | 2.32406E−10 | −3.16807E−09 | 5.89808E−11 | 1.62702E−10 | 4.09715E−11 |
| log(Reaction 2.k2) | −3.16807E−09 | 7.47680E−07 | 7.06745E−09 | 9.12989E−10 | −8.36361E−09 |
| log(Reaction 3.k3) | 5.89808E−11 | 7.06745E−09 | 2.32133E−09 | −3.87151E−10 | 2.90801E−10 |
| log(Reaction 4.k4) | 1.62702E−10 | 9.12989E−10 | −3.87151E−10 | 7.97490E−10 | −1.40968E−10 |
| log(Reaction 5.k5) | 4.09715E−11 | −8.36361E−09 | 2.90801E−10 | −1.40968E−10 | 2.83969E−10 |

### Synthesizing Heterotrifunctional Linker 17

The inventors next have synthesized a non-limiting example of heterotrifunctional linker 17 that bears a covalent inhibitor for each of the three enzyme-inhibitor pairs described herein in the present disclosure—SnapTag, cutinase, and CrabTag. The method of Synthesizing heterotrifunctional linker 17 is detailed in Example 6 below of the present disclosure.

### Preparing Branched Megamolecule Product 18 Using Heterotrifunctional Linker 17

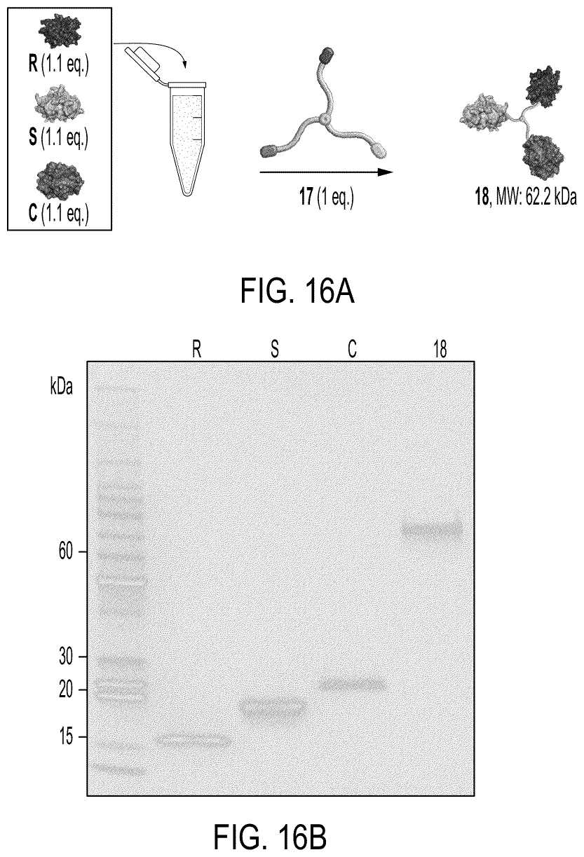

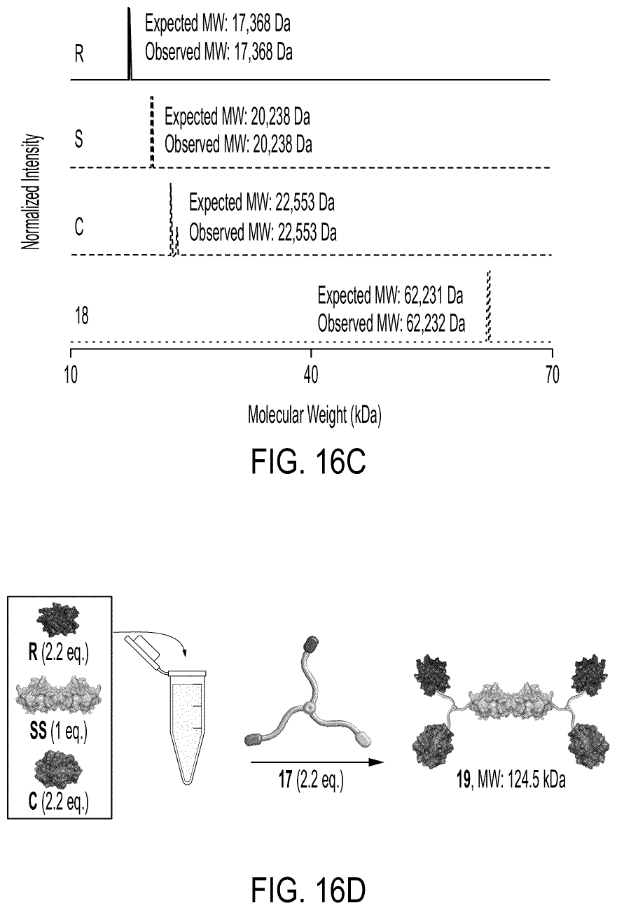

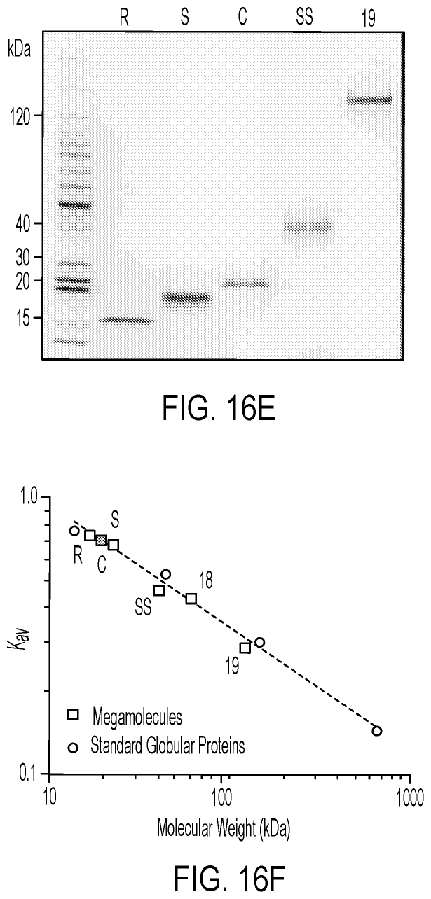

The present disclosure provides a method for preparing branched megamolecule product 18 using heterotrifunctional linker 17. The method comprises adding linker 17 (20 μM in PBS, 25° C.) into an equimolar solution of three monomeric enzymes (each at 22 μM) resulted in a one-pot reaction where only one copy of each enzyme has been simultaneously and, importantly, site-selectively localized to the linker core to form branched megamolecule product 18, as illustrated in FIG. 16A. The inventors have purified the major reaction peak using SEC and compared homogeneous product 18 to the starting enzymes using SDS-PAGE and ESI-MS, as illustrated in FIGS. 16B and 16C. The exact molecular weight of branched megamolecule product 18 is measured to be 62,232 Da, which is in excellent agreement to the expected molecular weight of 62,231 Da (ΔMW=1 Da).

As used herein, the terms “multi-functional linker #”, “heterotrifunctional linker #” and “linker #” are used interchangeably in the disclosure. The terms “compound having a structure of Formula #”, “compound #”, and “product #” are used interchangeably in the disclosure. For example, the terms “compound having a structure of Formula 1” and “product 1” are used interchangeably. The terms “compound having a structure of Formula 10”, “product 10”, “inhibitor having a structure of Formula 10”, “linker 10”, “covalent inhibitor 10”, “synthetic inhibitor 10”, “synthetic retinoid 10”, “compound 10”, and “synthetic inhibitor having a structure of Formula 10” are used interchangeably in the disclosure. The terms “compound having a structure of Formula 11”, “product 11”, “inhibitor having a structure of Formula 11”, “linker 11”, “covalent inhibitor 11”, “fluorescent linker 11”, and “synthetic inhibitor having a structure of Formula 11” are used interchangeably in the disclosure. The terms “megamolecule having a structure of Formula #”, “product #”, “megamolecule #”, and “megamolecule having a structure of Configuration #” are used interchangeably in the disclosure. The terms “π-donor 4”, “product 4”, and “compound having a structure of Formula 4” are used interchangeably in the disclosure. The terms “π-acceptor 7”, “product 7”, and “compound having a structure of Formula 7” are used interchangeably in the disclosure.

### Preparing Double-Branched Megamolecule 19 Using Heterotrifunctional Linker 17

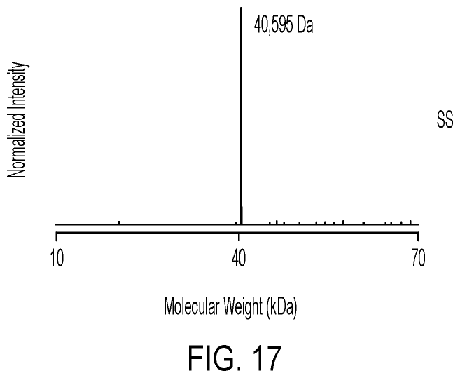

Finally, the present disclosure provides a method for the preparation of double-branched megamolecule 19 using heterotrifunctional linker 17 in a one-pot assembly involving six reactions of four molecules, as illustrated in FIG. 16D. Assembly of megamolecule 19 has been accomplished by reacting linker 17 (22 μM in PBS, 25° C.) with a mixture of both monomeric CrabTag and cutinase domains (both at 22 μM) and a di-SnapTag fusion protein (SS, 10 μM) core, as illustrated in FIG. 17. Purification of product 19 by SDS-PAGE shows a product band near 120 kDa, which is consistent with the predicted molecular weight of approximately 124.5 kDa, as illustrated in FIG. 16E. SEC has been used to characterize each of the enzyme starting materials, single-branched product 18, and double-branched product 19. The calculated partition coefficients (Kay) for all proteins and megamolecules have matched their expected molecular weights by alignment to the standard globular protein calibration curve (dotted line), indicating that each species had a single, globular structure, as illustrated in FIG. 16F. These results demonstrate the orthogonality of the enzyme-inhibitor reactions and their application to the one-pot assembly of complex products without the use of protecting groups.

The present disclosure has established a new reaction pair for assembly of megamolecules. The CrabTag domain efficiently reacts with the synthetic inhibitor 10 due to the high affinity of the synthetic retinoid and the rapid covalent reaction of the arylfluorosulfate electrophile with the tyrosine nucleophile. The CrabTag protein domain is monomeric, easily expressed in E. coli due to its small size (17 kDa), and is also significant because it is a human-derived enzyme. Further, this inhibition reaction has an effective rate constant of approximately 3,600 M⁻¹s⁻¹ and the enzyme-inhibitor complex is stable (>95% bound) at room temperature for over two weeks. Finally, the orthogonality of this reaction relative to the other two enzyme-inhibitor reactions used here have allowed the inventors to perform single pot assemblies of complex products, demonstrating its utility for convergent megamolecule assembly.

### Benefits of the Presently Disclosed Method of Assembling the Megamolecules

The advantages of the method to prepare the megamolecules of present disclosure is the ability to have batch-to-batch consistency of product formation, enable rapid synthesis of complex high molecular weight structures with exact placement of different types of bio-matters, such as antibody fragments, proteins, enzymes and peptides, and offer specificity of targeting a diseased tissue using a human-derived bio-matters (enzymes) and thus limiting immunogenicity.

The inventors believe that this new reaction of the present disclosure will play a significant role in the assembly of therapeutic antibody mimics, including antibody-drug conjugates, multi-specific antibodies, and hypervalent protein dendrimers. In each of these applications, it is critical that the assembly of several fragments results in a homogeneous target structure without the necessity for intermediate protecting and deprotecting reactions. Further, it is important that the enzymes are human-derived to minimize unwanted immunogenicity. The present disclosure advantageously provides a modular toolbox with the several, orthogonal enzyme-inhibitor reactions described herein will enable efficient, one-pot assembly of a broad range of megamolecule architectures.

### EXAMPLES

### Example 1

### Synthesis of the CRABP2 Synthetic Retinoid Covalent Inhibitor.

### Materials:

All chemicals involved in synthesis of target compounds were reagent grade unless stated otherwise.

DNase, isopropyl thiogalactoside (IPTG), 4-(4,6-dimethoxy-1,3,5-triazin-2-yl)-4-methyl-morpholinium chloride (DMTMM), 4-methylmorpholine (NMM), 1,8-Diazabicyclo(5.4.0)undec-7-enediisopropylcarbodiimide (DBU), dimethyl sulfoxide (DMSO), methyl 4-chloro-4-oxobutyrate, 3,3-dimethylacryloyl chloride, borane dimethyl sulfide complex, 4-iodo aniline, acetyl chloride, 4-iodo phenol, trimethylsilyl-acetylene, potassium fluoride, 6-[Fluorescein-5(6)-carboxamido]hexanoic acid, triethylamine, [4-(acetylamino)phenyl]imidodisulfuryl difluoride (AISF), aluminum trichloride, copper (I) iodide, and dichlorobis(triphenylphosphine)palladium (II) were purchased from Sigma-Aldrich.

2-chlorotrityl chloride synthesis resin was purchased from AnaSpec. Azido-PEG11-amine was purchased from Broadpharm.

Magnesium sulfate, sodium hydroxide, sodium acetate, sodium azide, sodium chloride, sodium bicarbonate, 2×YT media, kanamycin, and all other organic solvents were purchased from Thermo Fisher Scientific.

All primers and DNA block segments involved in cloning protein inserts were purchased from Integrated DNA Technologies (IDT) with standard desalting as means of purification.

The inventors used a pET28-b(+) expression vector, Q5 Hot Start Master Mix 2×, T4 DNA ligase, Golden Gate Master Mix (BsaI-HF v2), DH5a E. coli, and T7 SHuffle Express E. coli chemically competent cells were purchased from New England Biolabs (NEB).

Talon cobalt IMAC resin was purchased from Clontech. Qiaprep Miniprep Spin kits were purchased from Qiagen.

10× Phosphate-Buffered Saline (PBS), a 20×0.5 mL and 12×1.5 mL microtube block thermal mixer was purchased from Thermo Fisher Scientific.

### Experimental Procedures

The covalent inhibitor of the present disclosure comprise a highly-conjugated, linear backbone and a functional group such as rylfluorosulfate attached to an end of the highly-conjugated, linear backbone. The design of the covalent inhibitor was based covalent inhibition of CRABP2, at an active site tyrosine residue, by the arylfluorosulfate electrophile. This sulfur fluoride exchange (SuFEx) click reaction was activated by two proximal arginine residues of CRABP2 (Arg112 and Arg133) that electrostatically stabilized the reactive sulfur fluoride bond through the formation of a sulfate ester, lowering the pKa of the nucleophile and accelerating the inhibition reaction. The highly-conjugated, linear backbone further has high affinity for CRABP2 and thus facilitates the binding of inhibitor into the hydrophobic ligand binding pocket of the protein. The inventors prepared the synthetic retinoid with a modification at the hydrophobic region for ligation onto the linkers. The polar region of the retinoid displays the necessary arylfluorosulfate group for covalent modification by SuFEx with the monomeric CRABP2 active site tyrosine nucleophile as shown in FIG. 2.

The inventors have prepared a non-limiting covalent inhibitor 10 in a 10-step convergent synthesis through the conjugation of π-donor 4 and π-acceptor 7, as detailed in Scheme 1 below. The former was synthesized from 4-iodoaniline by acylation (1) and Friedel-Crafts cyclization (2) followed by reduction with borane dimethyl sulfide complex to yield tetrahydroquinoline 3. Acylation with 4-chloro-4-oxobutyrate gave para-iodoaniline derivative 4. The π-acceptor partner 7 was synthesized following an approach reported by Freccero. 4-Iodophenol was protected by 0-acetylation (5), coupled to trimethylsilylacetylene by Sonogashira cross-coupling (6), and desilylated with potassium fluoride. The protected synthetic retinoid 8 was then conjugated through Sonogashira coupling of r-acceptor (7) and π-donor (4), followed by saponification to reveal non-covalent retinoid inhibitor 9 with a succinic acid handle for chemical ligation. The inventors have completed the synthesis by sulfonylation of the terminal phenol with [4-(acetylamino)phenyl]imidodisulfuryl difluoride (AISF) to give synthetic retinoid 10.

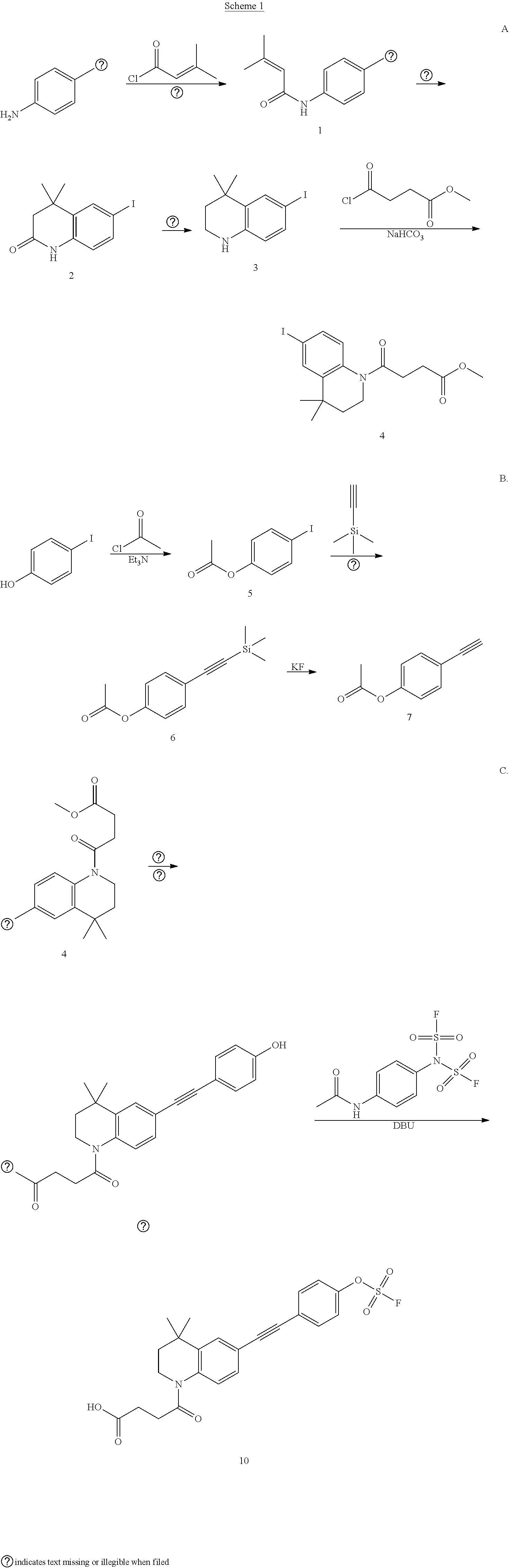

### Chemical Inhibitor Synthesis

The following protocol describes the convergent synthesis of the synthetic retinoid for targeted covalent inhibition of cellular retinoic acid binding protein 2 (CRABP2).

### N-(4-Iodophenyl)-3-methyl-2-butenamide, 1

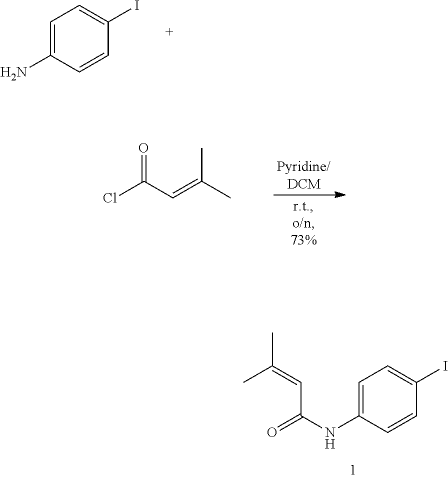

A solution of 4-iodoaniline (50 g, 1 eq.) was prepared in 800 mL DCM. 19.4 mL of pyridine was added at room temperature and the mixture was stirred for 30 minutes. The mixture was then put on ice and 26.72 mL of 3,3-dimethylacryloyl chloride was added dropwise. The reaction was then returned to room temperature and stirred for about 1-50 hours, about 6-20 hours, about 10-18 hours, about 12-16 hours, or about 16 hours. At the end of the reaction, the mixture was diluted with H₂O and extracted with EtOAc three times. The organic later was washed with 1 M HCl (aq.), sat. NaHCO₃ (aq.), and then with brine. The mixture was then dried with Na₂SO₄, filtrated, and concentrated. The pure product 1 (166.4 g, 73%) was obtained as a white powder by recrystallization in EtOH. ¹H NMR (500 MHz, DMSO) δ 9.91 (s, 1H), 7.62 (s, 2H), 7.45 (s, 2H), 5.84 (s, 1H), 2.14 (s, 3H), 1.86 (s, 3H). ¹³C NMR (126 MHz, DMSO) δ 164.81, 152.09, 139.53, 137.41, 121.34, 119.11, 86.30, 27.21, 19.68. MALDI-MS m/z, 302.0010 (M+H) calculated for C₁₁H₁₃INO 302.12.

### 6-iodo-4,4-dimethyl-3,4-dihydroquinolin-2(1H)-one, 2

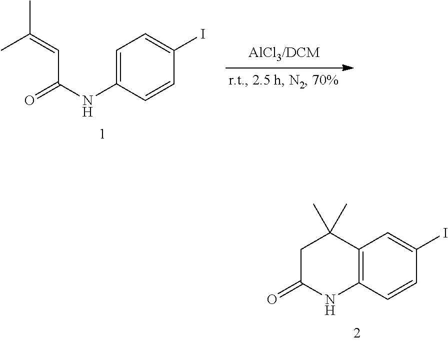

5 g (16.6 mmol, 1 eq.) of 1 and 3.33 g AlCl₃ (1.5 eq.) were combined, a rubber septum was added to the flask, and the flask was evacuated 3× with N₂. The mixture was then dissolved in 65 mL DCM. The reaction was stirred at room temperature for 2.5 hours before being diluted with 200 mL H₂O and stirred for 10 additional minutes. To the mixed solution was added 200 mL of 1.25 M NaOH (aq.). The organic fraction was then washed with H₂O and brine before drying with Na₂SO₄, filtration, and concentration on a rotary evaporator. The product, 2 (3.5 g, 70%), was purified as a white powder by recrystallization in EtOH. ¹H NMR (500 MHz, DMSO) δ 10.38 (s, 1H), 7.71 (s, 1H), 7.64 (s, 1H), 6.86 (s, 1H), 2.66 (s, 2H), 1.37 (s, 6H). ¹³C NMR (126 MHz, DMSO) δ 171.33, 139.03, 137.97, 137.07, 134.87, 119.95, 87.91, 46.66, 35.80, 29.16. MALDI-MS m/z, 301.9906 (M+H) calculated for C₁₁H₁₃INO 302.12.

### 6-iodo-4,4-dimethyl-1,2,3,4-tetrahydroquinoline, 3

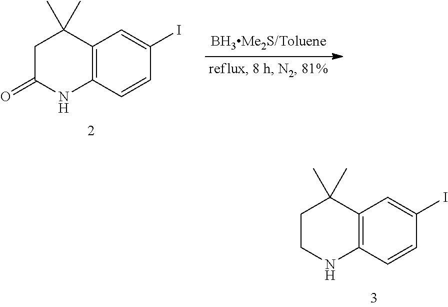

To a flask was added 2 (5.23 g, 1 eq.) and 39.2 mL toluene. The flask was evacuated with N₂ 3× and 10.974 mL of borane dimethyl sulfide complex at 2M in THE was added to the mixture. The reaction was performed at reflux for 8 hours. After the completion of the reaction, the mixture was allowed to stir for 30 min at room temperature before the addition of 33 mL of 10% Na₂CO₃ (aq.). The mixture was extracted with EtOAc, washed once with H₂O, and washed 3× with brine. The organic layer was dried with Na₂SO₄, filtered, and concentrated. The crude mixture was purified using silica chromatography (10:90 EtOAc:Hexanes+1% Et₃N) and concentrated to reveal a pure yellow oil product, 3 (4.2 g, 81%). The inventors note that the mixture should be stored in a dark, cool place (4° C. fridge recommended). ¹H NMR (500 MHz, DMSO) δ 7.29 (s, 1H), 7.09 (s, 1H), 6.29 (s, 1H), 5.94 (s, 1H), 3.17 (s, 2H), 1.56 (s, 2H), 1.19 (s, 6H). ¹³C NMR (126 MHz, DMSO) δ 143.75, 134.29, 133.50, 131.54, 115.83, 75.12, 36.67, 35.73, 31.25, 30.14. LC-TOF ESI-MS m/z, 288.0204 (M+H) calculated for C₁₁H₁₅IN 288.13.

### Methyl 4-(6-iodo-4,4-dimethyl-3,4-dihydroquinolin-1(2H)-yl)-4-oxobutanoate, 4

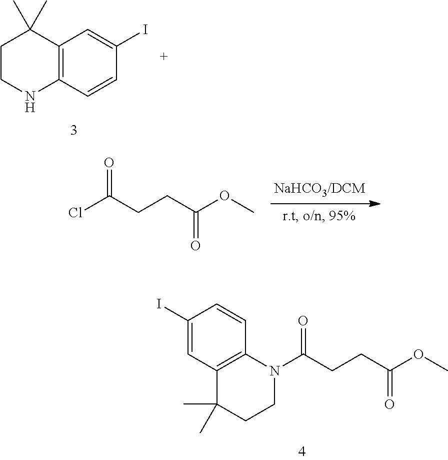

8.5 g (29.4 mmol, 1 eq.) of 3 was added to a flask with 432 mL DCM. 10 g of NaHCO₃ was then added to the solution and the mixture was stirred at room temperature for 30 minutes. The flask was placed on ice and methyl 4-chloro-4-oxobutyrate (4.39 mL) was added dropwise. The mixture was returned to room temperature and stirred for about 1-50 hours, about 6-20 hours, about 10-18 hours, about 12-16 hours, or about 16 hours. At the end of the reaction, the mixture was diluted with H₂O and the organic layer was washed with 10% Na₂CO₃ (aq.), sat. NH₄Cl (aq.), and brine before being dried with Na₂SO₄, filtered, and concentrated. The crude mixture was purified by silica chromatography (30:70 EtOAc:Hexanes) and concentrated. The pure product 4 (11.3 g, 95%) was collected as a clear oil. ¹H NMR (500 MHz, DMSO) δ 7.66 (s, 1H), 7.49 (s, 1H), 7.27 (s, 1H), 3.69 (s, 2H), 3.54 (s, 3H), 2.72 (s, 2H), 2.56 (s, 2H), 1.69 (s, 2H), 1.23 (s, 6H). ¹³C NMR (126 MHz, DMSO) δ 172.80, 170.75, 136.81, 134.33, 134.28, 127.01, 125.86, 124.80, 59.74, 51.28, 37.80, 33.14, 29.21, 14.08. MALDI-MS m/z, 402.0237 (M+H) calculated for C₁₆H₂₁INO₃ 402.25.

### 4-iodophenyl acetate, 5

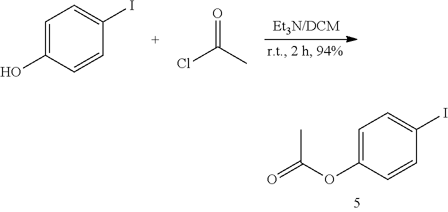

10 g (45.4 mmol, 1 eq.) of 4-iodophenol was added to 230 mL of DCM in a flask on ice. 9.468 mL of Et₃N was added into the flask and the mixture was stirred for 30 minutes. 3.872 mL of acetyl chloride was added dropwise to the mixture and the temperature was raised to room temperature before stirring for 2 hours. The mixture was diluted with 280 mL H₂O and the organic layer was washed 3× with brine. The organic later was then dried using Na₂SO₄, filtered, and concentrated on the rotary evaporator to yield a red oil as the crude mixture. The mixture was purified using silica chromatography (10:90 EtOAc:Hexanes) and concentrated to give product 5 (11.2 g, 94%) as a clear oil. ¹H NMR (500 MHz, DMSO) δ 7.79-7.72 (m, 2H), 7.01-6.94 (m, 2H), 2.26 (s, 3H). ¹³C NMR (126 MHz, DMSO) δ 169.44, 150.85, 138.68, 124.91, 90.97, 60.22, 40.51, 40.43, 40.34, 40.26, 40.17, 40.01, 39.84, 39.67, 39.50, 21.30, 21.24, 14.56. LC-TOF ESI-MS m/z, 263.2154 (M+H) calculated for C₈H₈IO₂ 263.05.

### 4-((trimethylsilyl)ethynyl)phenyl acetate, 6

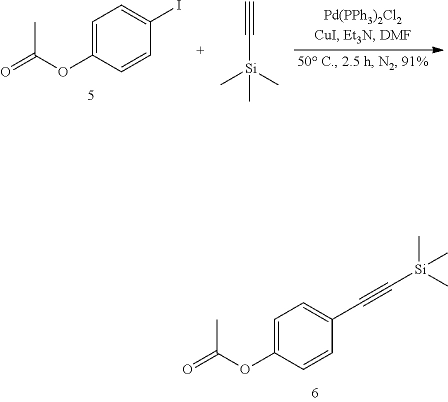

To a flask was added 569.4 mg (2.173 mmol, 1 eq.) of 5, 48.86 mg of Pd(PPh₃)₂Cl₂, and 24.7 mg CuI. The flask was the purged 5× with N₂ and 3.528 mL of DMF was added before the temperature of the flask was raised to 50° C. The mixture was stirred and to it was added 338.7 μL of trimethylsilyl-acetylene and 1.129 mL of Et₃N. The reaction proceeded for 2.5 hours and was then brought to room temperature before dilution in water. The mixture was extracted with EtOAc and washed 3× with brine. The organic later was then dried using Na₂SO₄, filtered, and concentrated by rotovap. The pure product, 6, was purified by silica chromatography (10:90 EtOAc:Hexanes) and concentrated to yield a yellow-orange solid (459 mg, 91%). ¹H NMR (500 MHz, CDCl₃) δ 7.27 (s, 2H), 6.84 (s, 2H), 2.10 (s, 3H), 0.05 (s, 9H). ¹³C NMR (126 MHz, CDCl₃) δ 169.11, 150.67, 133.19, 121.59, 120.89, 104.25, 94.37, 77.33, 77.08, 76.82, 21.19, 0.22, 0.00, −0.23, −0.45. LC-TOF ESI-MS m/z, 233.1627 (M+H) calculated for C₁₃H₁₇O₂Si 233.36.

### 4-ethynylphenyl acetate, 7

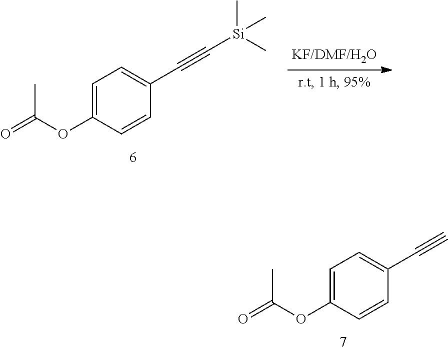 [SMILES: CC(=O)Oc1ccc(C#C[Si](C)(C)C)cc1 ; C#Cc1ccc(OC(C)=O)cc1]

300 mg (1.295 mmol, 1 eq.) of 6 was added to a mixture of 13.271 mL DMF and 13.271 mL H₂O. To the mixture was added 120 mg potassium fluoride and the solution was stirred for one hour at room temperature. The mixture was then diluted with 6.636 mL H₂O and extracted 3× with EtOAc. The organic fractions were combined and washed with brine 5× before being dried with Na₂SO₄, filtered, and concentrated to yield a brown oil. The crude oil was purified by silica chromatography (10:90 EtOAc:Hexanes) and concentrated to yield an off-white solid as the pure product, 7 (196 mg, 95%). ¹H NMR (500 MHz, CDCl₃) δ 7.42 (s, 2H), 7.00 (s, 2H), 2.99 (s, 1H), 2.23 (s, 3H). ¹³C NMR (126 MHz, CDCl₃) δ 169.09, 150.87, 133.34, 121.71, 119.79, 82.82, 77.30, 77.28, 77.03, 76.77, 21.13. LC-TOF ESI-MS m/z, 161.3268 (M+H) calculated for C₁₀H₉O₂ 161.18.

### Methyl 4-(6-((4-(acetyloxy)phenyl)ethynyl)-4,4-dimiethyl-3,4-dihydroquinolin-1(2H)-yl)-4-oxobutanoate, 8

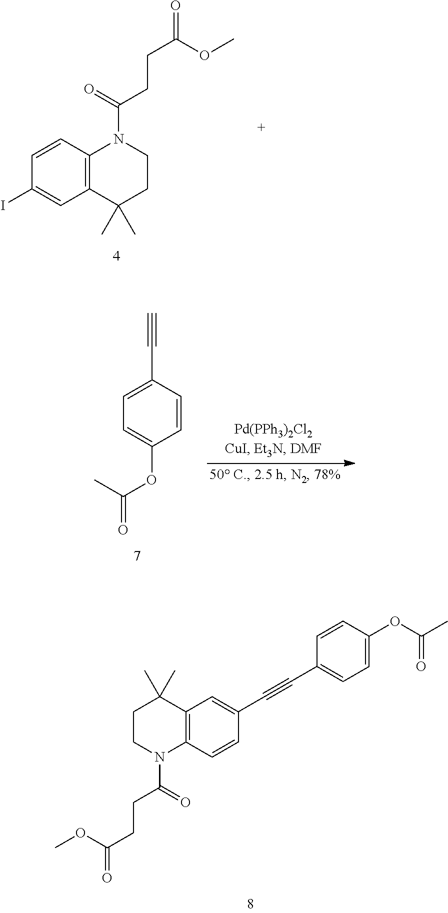 [SMILES: COC(=O)CCC(=O)N1CCC(C)(C)c2cc(I)ccc21 ; C#Cc1ccc(OC(C)=O)cc1 ; COC(=O)CCC(=O)N1CCC(C)(C)c2cc(C#Cc3ccc(OC(C)=O)cc3)ccc21]

330.3 mg (824 μmol, 1 eq.) of 4, 18.54 mg of Pd(PPh₃)₂Cl₂, and 9.34 mg CuI. The flask was the purged 5× with N₂. The temperature of the flask was then raised to 50° C. 1.489 mL of DMF was stirred in followed by the addition of 145 mg (1.1 eq.) 7 and 477.1 μL of Et₃N. The reaction occurred over 2.5 hours and was then brought to room temperature before dilution with water. The mixture was extracted with EtOAc and washed 3× with brine. The organic later was then dried using Na₂SO₄, filtered, and concentrated by rotary evaporator. The pure product, 8, was then purified by silica chromatography (25:75 EtOAc:Hexanes) and concentrated to yield an orange oil (198 mg, 78%). ¹H NMR (500 MHz, CDCl₃) δ 7.57 (s, 2H), 7.51 (s, 1H), 7.36 (s, 2H), 7.12 (s, 2H), 3.85 (s, 2H), 3.70 (s, 3H), 2.83 (s, 2H), 2.73 (s, 2H), 2.33 (s, 3H), 1.82 (s, 2H), 1.35 (s, 6H). ¹³C NMR (126 MHz, CDCl₃) δ 173.32, 171.16, 169.16, 150.47, 137.23, 132.73, 129.25, 129.22, 124.78, 121.72, 120.95, 89.32, 88.40, 77.28, 77.02, 76.77, 60.41, 51.80, 41.69, 38.36, 34.68, 33.58, 31.60, 29.97, 29.63, 29.29, 22.67, 21.16, 21.06, 14.21, 14.13. MALDI-MS m/z, 434.1969 (M+H) calculated for C₂₆H₂₈NO₅ 434.50.

### 4-(6-((4-hydroxyphenyl)ethynyl)-4,4-dimethyl-3,4-dihydroquinolin-1(2H)-yl)-4-oxobutanoic acid, 9

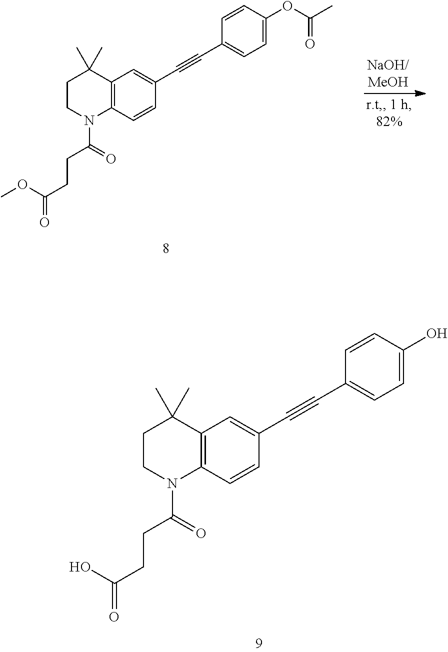 [SMILES: COC(=O)CCC(=O)N1CCC(C)(C)c2cc(C#Cc3ccc(OC(C)=O)cc3)ccc21 ; CC1(C)CCN(C(=O)CCC(=O)O)c2ccc(C#Cc3ccc(O)cc3)cc21]

6.24 g (14.41 mmol, 1 eq.) of the compound having the structure of Formula 8 was mixed in a flask with 57.64 mL MeOH. 57.64 mL of 1 M NaOH (aq.) was then added to the flask and the solution was stirred at room temperature for one hour. The mixture was acidified with 232 mL of 0.5 M HCl and diluted with EtOAc. The organic layer was washed with H₂O, brine, and dried using Na2SO₄ before filtration and concentration by rotovap. The crude mixture was then purified using silica chromatography (80:20 EtOAc:Hexanes+1% AcOH) and concentrated. The pure product was then washed and concentrated 3× using toluene to remove excess AcOH. The inventors note that the product will attempt to foam during concentration so caution must be taken during the purification process. The product, 9 (4.5 mg, 82%), was purified a brown solid. ¹H NMR (500 MHz, CD₃OD) δ 7.60 (s, 1H), 7.45 (s, 1H), 7.32 (s, 4H), 6.75 (s, 2H), 3.78 (s, 2H), 3.26 (s, 1H), 2.76 (s, 2H), 2.61 (s, 2H), 1.77 (s, 2H), 1.28 (s, 6H). ¹³C NMR (126 MHz, CD₃OD_SPE) δ 174.92, 172.38, 171.57, 157.76, 136.53, 132.69, 131.65, 128.58, 128.41, 124.84, 115.13, 113.89, 89.14, 86.71, 60.14, 48.14, 47.97, 47.80, 47.63, 47.46, 47.29, 47.12, 41.58, 38.08, 36.04, 34.38, 34.29, 33.11, 31.36, 29.47, 29.06, 28.77, 28.40, 24.82, 22.31, 19.65, 19.49, 17.77, 13.09, 13.06, 10.39. LC-TOF ESI-MS m/z, 378.1773 (M+H) calculated for C₂₃H₂₄NO₄ 378.42.

### 4-(6-((4-((fluorosulfonyl)oxy)phenyl)ethynyl)-4,4-dimethyl-3,4-dihydroquinolin-1(2H)-yl)-4-oxobutanoic acid, 10

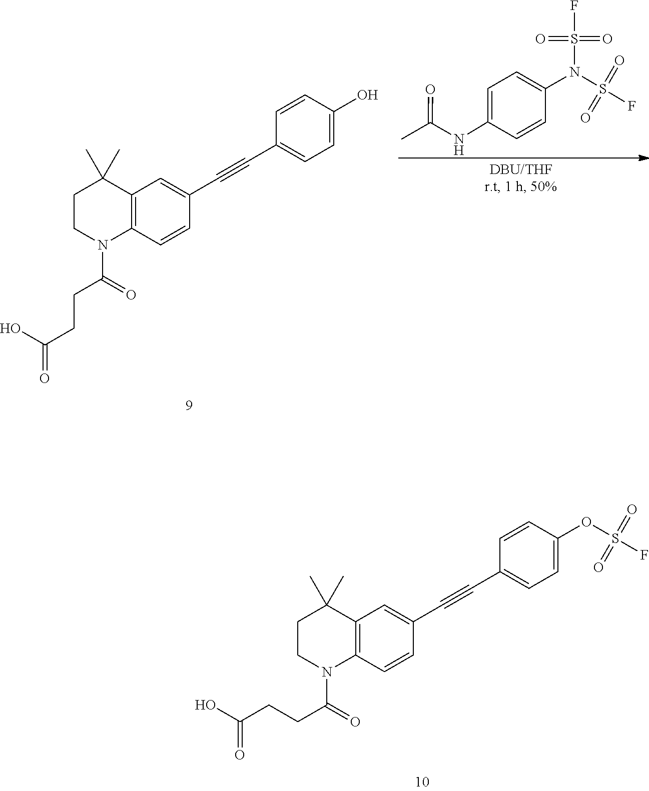 [SMILES: CC1(C)CCN(C(=O)CCC(=O)O)c2ccc(C#Cc3ccc(O)cc3)cc21 ; CC(=O)Nc1ccc(N(S(=O)(=O)F)S(=O)(=O)F)cc1 ; CC1(C)CCN(C(=O)CCC(=O)O)c2ccc(C#Cc3ccc(OS(=O)(=O)F)cc3)cc21]

1.9165 g (5.083 mmol, 1 eq.) of 9 was dissolved in 25.41 mL of THF and to the mixture was added 1.713 g of [4-(acetylamino)phenyl]imidodisulfuryl difluoride (AISF). Over a 30 second period, 1.681 mL of DBU was added dropwise to the reaction mixture. The reaction was stirred at room temperature for one hour. At the completion of the reaction, the mixture underwent a 10:1 dilution in EtOAc and the organic layer was washed 3× with 0.5 M HCl (aq.), 2× with sat. HNaCO₃ (aq.), and finally acidified by washing 3× with 0.5 M HCl (aq.). The organic layer was washed with brine 3× before drying with Na₂SO₄, filtered, and concentrated. The crude mixture was purified by silica chromatography (10:90 MeOH:DCM) and then the product was concentrated using the rotovap. The final product, 10 (1.16 g, 50%), was recovered as a white-pink solid. ¹H NMR (500 MHz, CDCl₃) δ 7.62 (d, J=8.4 Hz, 2H), 7.51 (s, 1H), 7.33 (d, J=8.5 Hz, 4H), 3.83 (s, 2H), 2.79 (s, 2H), 2.77 (s, 2H), 1.79 (s, 2H), 1.32 (s, 6H). ¹³C NMR (126 MHz, CD₃CN) δ 177.92, 171.55, 149.61, 137.83, 133.79, 129.69, 129.34, 128.53, 125.61, 125.18, 124.61, 121.40, 119.45, 91.56, 87.49, 42.12, 38.54, 33.85, 30.10, 29.51. ¹⁹F NMR (376 MHz, CDCl₃) δ 38.08. LC-TOF ESI-MS m/z, 460.1323 (M+H) calculated for C₂₃H₂₃FNO₆S 460.49.

### Example 2

### Engineering CRABP2

The inventors have constructed a T7 expression plasmid incorporating the sequence for CRABP2 (referred to hereon as CrabTag) modified with a C-terminal His-tag. The inventors transformed the plasmid into chemically competent E. coli and purified the protein with immobilized metal affinity chromatography (IMAC).¹⁸ The inventors reacted the protein (10 μM) in phosphate buffered saline (PBS; 2.7 mM KCl, 138 mM NaCl, pH 7.4) with covalent inhibitor 10 (1.1 equivalents) and purified the adduct using size exclusion chromatography (SEC). Electrospray ionization mass spectrometry (ESI-MS) confirmed presence of the adduct as shown in FIGS. 3 and 4.

### Example 3

Reacting CRABP2 Protein with Synthetic Retinoid Inhibitor 10 to Form CrabTag-10 Adduct and its Characteristics

The inventors have grown crystals of the inhibited enzyme and obtained an X-ray structure that diffracted to 1.8 Å (Rwork=0.21, Rfree=0.25) as shown in FIG. 5. The electron density map shows that the synthetic retinoid inhibitor forms a covalent sulfate ester adduct at the polar region of the inhibitor with the nucleophilic residue, Tyr135 as shown in FIGS. 6-9. A structural alignment comparison of the CRABP2 protein backbone in our CrabTag-10 adduct with the non-covalent CRABP2-synthetic retinoid complex from Chrisholm et al. (PDBID: 6HKR) shows high similarity (RSMD=1.18 Å over all atoms without refinement) as shown in Table 1 above. Consistent with the covalent CRABP2-diarylsulfate crystal structure from Chen et al., synthetic retinoid inhibitor 10 selectively modifies Tyr135 polar stabilization interactions between the arylfluorosulfate electrophile. Further, the proximal hydrogen bonding donor (Arg112 and Arg133) side chains help to facilitate the covalent SuFEx modification of the Tyr135 residue.

### Example 4

### Synthesis of Linker 11

The inventors synthesized linker 11, which included a fluorophore to characterize the kinetics for reaction of the covalent inhibitor and CrabTag as shown in FIG. 10A The inventors ran parallel reactions that varied the concentration of the linker (2, 5, 10, 20, 40, 65, and 100 μM) at a constant concentration of CrabTag (1 μM) and stopped the reactions using Laemmli reducing buffer at times ranging from 0 to 120 seconds as shown in FIG. 10B. Intact mass spectrometry analysis of the protein-inhibitor complexes for the reactions of CrabTag with either inhibitor 10 or 11 reveal monomeric adducts with only one modification that corresponds to the mass of either inhibitor as shown in FIGS. 10C and 4. The inventors separated the reaction products with SDS-PAGE and quantitated the mean fluorescence intensity of protein bands that corresponded to the adduct with ImageJ. The inventors fit the kinetic data to a single-phase exponential decay to obtain observed rate constants, kobs as shown in FIG. 10D. The inventors then plotted the kobs values against the linker concentration to give a Michaelis-Menten curve, yielding the inactivation rate constant ki and the equilibrium binding affinity of the enzyme-inhibitor complex KI as shown in FIG. 10E. The ratio of these parameters gives the second order rate constant for the covalent modification reaction, keff, as (3.6±0.5)×10³ M⁻¹s⁻¹. The inventors also monitored the stability of the adduct by incubating the product (1 μM in PBS, 25° C.) and found that over 95% of the protein domains in solution retained covalent occupancy of the linker after two weeks, as illustrated in FIG. 11.

Trivalent linker (Tri-EG(11,11,11)—(CP,CP,pNPP)-1,3,5-benzenetricarboxanide) was prepared according to previously published procedures. Bivalent linkers (Bi-EG(11)-(pNPP, pNPP)) and (Bi-EG(11)-(CP, CP)) were prepared from previously reported procedures.² The synthesis of both bivalent linker 11 (Bi-EG(11,11)—(R,F)) and heterotrivalent linker 17 (Tri-EG(7,7,7)—(R, CP,pNPP)) is shown on the following pages.

### Bi-EG(11)-(CrabTag Ligand, Fluorescein), 11

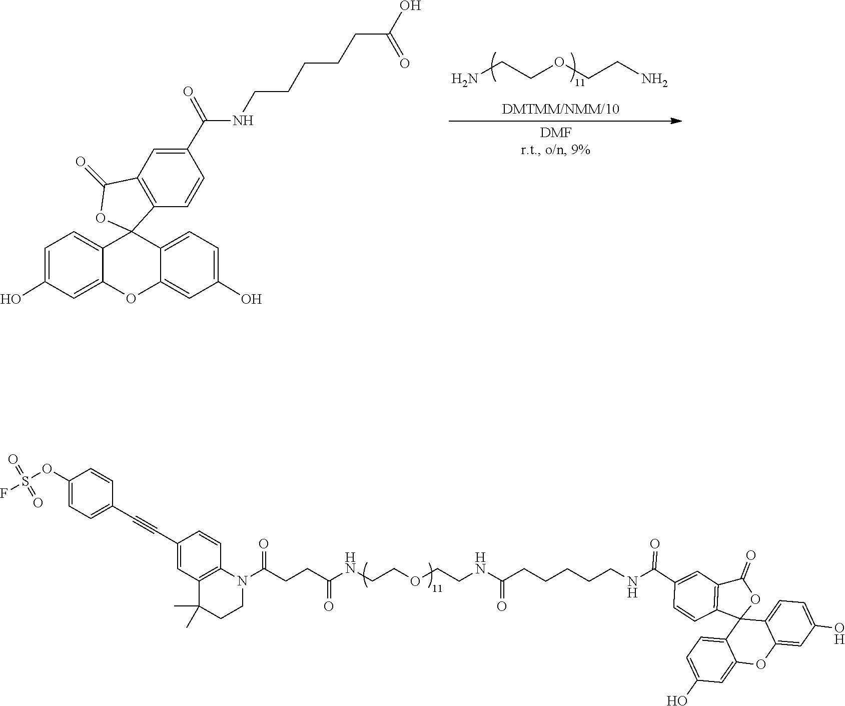

To a glass vial was added compound 10 (23.92 mg, 52 μmol), 25 mg (52 μmol) 6-[Fluorescein-5(6)-carboxamido]hexanoic acid, 18 mg DMTMM and 1 mL THF. After dissolving, 44 μL NMM was added to the mixture and stirred for 4 hours. The solvent was then removed by rotovap before adding DMF (450 μL), 24.3 mg (45 μmol, 22.3 μL) of the azido-PEG11-amine linker and NMM (16 μL, 144 mol). The reaction was then stirred for about 1-50 hours, about 6-20 hours, about 10-18 hours, about 12-16 hours, or about 16 hours. After this period, the solution was diluted to 5 mL with 9:1 DMSO:H₂O and purified on a C18 reversed phase column via HPLC using TFA as the mobile phase additive (giving the TFA impurity peak for the ¹⁹F NMR at δ −76.70). Product fractions were pooled and lyophilized to yield a yellow-green oil (5.97 mg, 9.1%). ¹H NMR (500 MHz, CD₃CN) δ 8.39 (s, 1H), 7.70 (d, J=8.0 Hz, 3H), 7.59 (d, J=8.9 Hz, 2H), 7.52 (t, J=13.3 Hz, 5H), 7.34 (d, J=8.7 Hz, 1H), 6.74 (d, J=2.6 Hz, 3H), 6.67 (d, J=8.6 Hz, 4H), 6.58 (dd, J=8.7, 2.5 Hz, 3H), 3.78 (d, J=6.4 Hz, 2H), 3.57 (s, 41H), 3.48 (d, J=5.2 Hz, 4H), 3.42 (t, J=6.1 Hz, 4H), 3.31 (s, 2H), 2.78 (d, J=6.5 Hz, 2H), 2.51 (s, 2H), 2.17 (t, J=7.3 Hz, 4H), 1.79 (d, J=6.3 Hz, 2H), 1.64 (p, J=7.3 Hz, 4H), 1.32 (s, 6H). ¹⁹F NMR (376 MHz, CD₃CN) δ 37.12, −76.70. MALDI-MS m/z, 1456.5924 (M+H) calc'd for C₇₅H₉₃FN₄O₂₃S 1457.6253.

### Example 5

### Assembling Megamolecules Using a Heterotrifunctional Linker Bearing Three Inhibitors

The inventors next demonstrate that the inventors can assemble megamolecules using three reactions—CrabTag with 10, cutinase with a PNPP inhibitor, and SnapTag with a CP inhibitor.² Importantly, the inventors first confirmed that each enzyme only reacts with its paired covalent inhibitor as shown in FIG. 19 and therefore expected that branched megamolecule 16 could be assembled in a single, one-pot reaction from four reactants: cutinase, a SnapTag-CrabTag (SR) fusion protein, fluorescent linker 11, and a heterotrifunctional linker bearing two inhibitors for SnapTag and one inhibitor for cutinase.

There are 120 discreet pathways that could give product 16. Consideration of the relative rates for the reactions of the three enzymes narrows the possible pathways to the two as shown in FIG. 12A.

Because the reaction of CrabTag with its inhibitor is the fastest, the inventors expected that the SR fusion protein would first react with fluorescent linker 11 to give intermediate 12. Based on the relative rates for SnapTag and cutinase reactions with their inhibitors, the inventors expected that intermediate 13 would form next. In the next step, the inventors predicted that two reaction pathways—forming either intermediates 14 or 15—would compete to give final product 16. After performing the experiment, the inventors obtained time-resolved quantification of reaction intermediates and formation of final product 16 using both fluorescent and Coomassie stained SDS-PAGE as shown in FIG. 12B.

By monitoring the concentration of each intermediate, the inventors can assess the kinetics and determine the abundance of each reaction intermediate over time as shown in FIG. 12C. The inventors used the SimBiology application within MATLAB to develop a simple block diagram to model the batch reaction for the megamolecule assembly as illustrated in FIG. 13. Due to the fast reaction kinetics of CrabTag inhibition, a key assumption to our model was a pseudo-steady state hypothesis on the concentration of fluorescent linker 11. Using a non-linear regression as our statistical model, the inventors simultaneously solved the system of differential rate law equations and estimated the second-order kinetic rate constant parameters from the model as illustrated in FIGS. 13-15 and Tables 2-5. This experiment revealed that a mechanistic approach could be used to describe and monitor the stepwise assembly of an atomically precise multi-protein scaffold over time.

### Example 6

### Synthesizing Heterotrifunctional Linker 17

The inventors next synthesized heterotrifunctional linker 17 that bears a covalent inhibitor for each of the three enzyme-inhibitor pairs described in this work—SnapTag, cutinase, and CrabTag as detailed below. The method of preparing the multi-functional linker (Formula 17) is illustrated in Scheme 3 above.

### Tri-EG(7,7,7)—(R,CP,pNPP), 17

33 mg of 2-chlorotritylchloride resin (50 mol) was added into a 5 mL polypropylene reaction cartridge and the resin was then swelled for 1 hour in N-methylpyrrolidinone (NMP, 0.5 mL).

Once swelled, 21.8 mg of azido-EG7-amine (55 mol) and diisopropylethylamine (DIEA, 35 μL, 200 mol) were added to the cartridge and the mixture was then rotated on a shaker for about 1-50 hours, about 6-20 hours, about 10-18 hours, about 12-16 hours, or about 16 hours (about 16 hours). The mixture was then drained under vacuum and the resin was washed with dimethylformamide (DMF, 3×10 mL) followed by dichloromethane (DCM, 3×10 mL) before drying. The resin was resuspended in a mixture of 10% methanol:90% NMP (0.5 mL), followed by the addition of DIEA (35 μL, 200 μmol) and allowed to shake for about 1-50 hours, about 6-20 hours, about 10-18 hours, about 12-16 hours, or about 16 hours. The cartridge was once again drained, washed with DMF (3×10 mL) and DCM (3×10 mL), and dried. The azido group was reduced by treatment with trimethylphosphine (1M in THF, 0.25 mL, 250 mol) for 3 before being drained, washed with THE (5×10 mL), and dried. The resin was then treated with a mixture of 10% water:90% THF (2 mL) for about 1-50 hours, about 6-20 hours, about 10-18 hours, about 12-16 hours, or about 16 hours. The resin was then washed with THE (3×10 mL), DMF (3×10 mL) and DCM (3×10 mL) and dried. The resin was swelled in NMP (0.5 mL) for about 1-50 hours, about 6-20 hours, about 10-18 hours, about 12-16 hours, or about 16 hours before adding a mixture of 15 mg of diethyl 1,3,5-benzenetricarboxylate (55 mol), 8.91 mg of HOBT (66 mol), and 12.6 mg EDC·HCl (66 mol) in 0.5 mL NMP. The resin was washed the next day with DMF (5×10 mL) and DCM (5×10 mL) before being dried. The resin was then suspended in EtOH (2 mL) and 6M NaOH (1.0 mL, 2M final) was added for 3 hours to saponify the diethyl functional groups into carboxylic acids derivatives. The solvent was removed using suction and the resin was washed with water (5×10 mL), 10% AcOH (aq.) (3×10 mL), water again (5×10 mL), DMF (5×10 mL), and DCM (5×10 mL) before drying with suction. To the resin was added 1 mL of NMP, 17.82 mg of HOBT (132 μmol), and 25.2 mg EDC·HCl (132 μmol). After 30 minutes of shaking, 43.6 mg of azido-EG7-amine (110 μmol) was added to the reaction mixture and reacted for about 1-50 hours, about 6-20 hours, about 10-18 hours, about 12-16 hours, or about 16 hours. The cartridge was drained and washed the next day with both DMF (5×10 mL) and DCM (5×10 mL) before drying. The resin was then treated once again with trimethylphosphine (1M in THF, 0.5 mL, 500 mol) for 3 hours before the mixture was drained and the resin was washed with THE (5×10 mL), followed by treatment with a 10% water:90% THE mixture (2 mL) for about 1-50 hours, about 6-20 hours, about 10-18 hours, about 12-16 hours, or about 16 hours. The resin was then washed with THF (3×10 mL), DMF (3×10 mL), and DCM (3×10 mL) and dried thoroughly. The resin was then swelled again in NMP (0.5 mL) for 30 minutes. During this time, 20.1 mg of the SnapTag inhibitor (CP, 55 μmol), 18.9 mg of the cutinase inhibitor (pNPP, 55 μmol), 17.82 mg of HOBT (132 μmol), and 25.2 mg EDC·HCl (132 mol) were added to a glass vial and suspended in NMP (1 mL) and stirred. The mixture was then added to the cartridge and shaken for about 1-50 hours, about 6-20 hours, about 10-18 hours, about 12-16 hours, or about 16 hours. The resin was washed with both DMF (5×10 mL) and DCM (5×10 mL), and then dried. The product was then cleaved from the resin using a 5% mixture of trifluoroacetic acid (TFA) in DCM (2×3 mL) for 2 hours. Excess TFA was removed by dilution with toluene and evaporation (3×) to yield a pink oil. 3.8 mg of CrabTag inhibitor 10 (8 mol) was added to a vial with 400 μL DMF, 1.4 mg HOBT (10 μmol), and 1.9 mg EDC·HCl (5 mol) and stirred for 30 minutes. The mixture was then added to the oil (12 mg, 6.2 μmol) with 6 eq. of NMM (6.62 μl, 60 μmol) and reacted for about 1-50 hours, about 6-20 hours, about 10-18 hours, about 12-16 hours, or about 16 hours. The solution was then diluted to 5 mL with a 90%:10% DMSO:water mixture and purified on a C8 reversed phase column by HPLC. Product fractions were pooled and lyophilized to yield a light champagne pink colored oil (1.1 mg, 8%). ¹H NMR (400 MHz, CDCl3) δ 8.59 (s, 1H), 8.53 (s, 1H), 8.25 (d, J=8.9 Hz, 1H), 7.80 (s, 1H), 7.65 (d, J=8.8 Hz, 1H), 7.51 (d, J=1.7 Hz, 1H), 7.41 (d, J=8.8 Hz, 2H), 7.34 (t, J=7.9 Hz, 4H), 4.46 (s, 1H), 4.06 (s, 1H), 3.83 (t, J=6.3 Hz, 1H), 3.66-3.60 (m, 36H), 3.45 (s, 3H), 2.87 (s, 1H), 2.74 (s, 1H), 2.62 (s, 1H), 2.38 (t, J=7.6 Hz, 1H), 2.21 (d, J=7.6 Hz, 2H), 1.28 (s, 70H), 1.12 (s, 2H), 0.97 (d, J=7.5 Hz, 4H), 0.92-0.89 (m, 8H), 0.88-0.81 (m, 13H), 0.75 (q, J=7.5 Hz, 4H). ¹⁹F NMR (376 MHz, CDCl3) δ 37.48. MALDI-MS m/z, 2397.9622 (M+Na+) calc'd for C₁₁₀H₁₆₁ClFN₁₂O₃₈PSNa 2398.01.

### Example 7

### Preparing Branched Megamolecule Product 18 Using Heterotrifunctional Linker 17

The inventors next synthesized heterotrifunctional linker 17 that bears a covalent inhibitor for each of the three enzyme-inhibitor pairs described in this work—SnapTag, cutinase, and CrabTag. Adding linker 17 (20 1 AM in PBS, 25° C.) into an equimolar solution of three monomeric enzymes (each at 22 1 AM) resulted in a one-pot reaction where only one copy of each enzyme was simultaneously and, importantly, site-selectively localized to the linker core to form branched megamolecule product 18 as illustrated in FIG. 16A. The inventors purified the major reaction peak using SEC and compared homogeneous product 18 to the starting enzymes using SDS-PAGE and ESI-MS as illustrated in FIG. 16B-C. The exact molecular weight of branched megamolecule product 18 (Configuration 18) was 62,232 Da, which is in excellent agreement to the expected molecular weight of 62,231 Da (ΔMW=1 Da).

Other megamolecules having the same central compound having the structure of Formula 20 with different synthetic inhibitor-enzyme pair can be prepared similarly according to the method discussed above for the megamolecule having the structure of Configuration 18.

### Example 8

### Preparing Double-Branched Megamolecule 19 Using Heterotrifunctional Linker 17

Finally, the inventors describe the preparation of double-branched megamolecule 19 using heterotrifunctional linker 17 in a one-pot assembly involving six reactions of four molecules as illustrated in FIG. 16D. Assembly of megamolecule 19 was accomplished by reacting linker 17 (22 1 AM in PBS, 25° C.) with a mixture of both monomeric CrabTag and cutinase domains (both at 22 1 AM) and a di-SnapTag fusion protein (SS, 10 1 AM) core, as illustrated in FIG. 17. Purification of product 19 by SDS-PAGE shows a product band near 120 kDa, which is consistent with the predicted molecular weight of approximately 124.5 kDa as illustrated in FIG. 16E. SEC was used to characterize each of the enzyme starting materials, single-branched product 18, and double-branched product 19. The calculated partition coefficients (Kav) for all proteins and megamolecules matched their expected molecular weights by alignment to the standard globular protein calibration curve (dotted line), indicating that each species had a single, globular structure as illustrated in FIG. 16F. These results demonstrate the orthogonality of the enzyme-inhibitor reactions and their application to the one-pot assembly of complex products without the use of protecting groups.

### Example 9

### Cloning of CRABP2 (R).

DNA constructs were developed with a library of golden gate ligation cloned fusion protein building blocks. The CRABP2 gene cassette was purchased from IDT based on a previously published sequence and was then PCR amplified using a forward primer containing a 5′ BsaI restriction site followed by the START codon. The reverse primer included a hexahistidine tag and the STOP codon. Both of these primers (SEQ ID NOS: 5-11) are provided in Table 6. This fragment was purified by agarose gel electrophoresis, digested with Bsal-HF v2 in a golden gate master mix (New England Biolabs), and ligated into a pET28-b(+) plasmid. This generated the N-CRABP2-H⁶—C protein construct (SEQ ID NO: 1) and the N—H⁶-TEV-CRABP2-C construct (SEQ ID NO: 2). The inventors transformed the sequence into chemically competent DH5a (New England Biolabs) E. coli and plated on LB agar with Kanamycin. The inventors transformed the sequence-verified the SR in pET28b into T7 SHuffle Express (New England Biolabs) E. coli as the expression strain. The N-CRABP2-H⁶—C protein construct has a sequence listed as SEQ ID NO: 1, a formula of C723H1150N2020220S6, a molecular weight of 16384.67 Da, and ε280 of 19605 M⁻¹ cm⁻¹. The N—H⁶-TEV-CRABP2-C construct has a sequence listed as SEQ ID NO: 2, a formula of C768H1212N2120233S7, a molecular weight of 17367.77 Da, and ε280 of 21095 M⁻¹cm⁻¹.

TABLE 6
Primer sequences (SEQ ID NOS: 5-11) for bacterial cell line cloning.

| | | | | Linker | |
|---|---|---|---|---|---|
| SEQ ID NO | Gene | Region | Direction | Type | Sequence |
| SEQ ID NO: 5 | SnapTag | N Terminal | Forward | N/A | TTTTGGTCTCaCATGGACAAAGACTGCGA |
| | | | | | AATGAAACG |
| SEQ ID NO: 6 | SnapTag | N Terminal | Reverse | XTEN | TTTTGGTCTCaTCAGAGGTACCCGGGGTT |
| | | | | | TCGCTACCAGAACCCAGACCCGGTTTAC C |
| SEQ ID NO: 7 | CRABP2 | N Terminal | Forward | N/A | TTTTGGTCTCaCATGCCGAACTTTAGCGG |
| | | | | | CAACTGG |
| SEQ ID NO: 8 | CRABP2 | N Terminal | Forward | HisTag + | TTTTGGTCTCaCATGCATCATCACCACCA |
| | | | | TEV | CCATGAAAACCTATACTTCCAAGGCCCGA |
| | | | | | ACTTTAGCGGCAACTGG |
| SEQ ID NO: 9 | CRABP2 | C Terminal | Forward | XTEN | TTTTGGTCTCaCTGAAAGCGCCCCGAACT |
| | | | | | TTAGCGGCAACTGG |
| SEQ ID NO: 10 | CRABP2 | C Terminal | Reverse | N/A | TTTTGGTCTCaCTTATTCACGAACATACAC |
| | | | | | ACGGGTGC |
| SEQ ID NO: 11 | CRABP2 | C Terminal | Reverse | His Tag | TTTTGGTCTCaCTTAATGGTGGTGGTGAT |
| | | | | | GATGTTCACGAACATACACACGGGTGC |

CRABP2 (R) Sequence, with C-terminal Histidine Tag (CRABP2-H6)
(SEQ ID NO: 1)
PNFSGNWKIIRSENFEELLKVLGVNVMLRKIAVAAASKPAVEIKQEGDTFYIKTSTTVRTTEI
NFKVGEEFEEQTVDGRPCKSLVKWESENKMVCEQKLLKGEGPKTSWTRELTNDGELILTM
TAD DVVCTRVYVREHHHHHH
CRABP2 (R) Sequence, with N-terminal Histidine Tag (Start-H6-TEV-CRABP2)
(SEQ ID NO: 2)
MHHHHHHENLYFQGPNFSGNWKIIRSENFEELLKVLGVNVMLRKIAVAAASKPAVEIKQEG
DTFYIKTSTTVRTTEINFKVGEEFEEQTVDGRPCKSLVKWESENKMVCEQKLLKGEGPKTS
WT RELTNDGELILTMTADDVVCTRVYVRE

### Example 10

### Cloning of SnapTag-CRABP2 (SR).

DNA constructs were made possible by the development of a library of golden gate ligation cloned fusion protein building blocks that incorporate thermostable SnapTag and CRABP2 domains. The SnapTag gene cassette was PCR amplified using a preexisting C-terminal SnapTag construct used in previously published work by Modica et al., The forward primer for the SnapTag gene cassette contained the start codon, as well as a BsaI restriction site for golden gate cloning. The reverse primer for this cassette contained the genetic XTEN linker. The CRABP2 gene cassette was PCR amplified from the CRABP2 construct by using a forward primer containing a 5′ BsaI restriction site followed by a segment encoding the remaining segment for the XTEN linker. The reverse primer for this gene cassette included a hexahistidine tag and the STOP codon. Both sets of primers used are provided in Table 6. The method for generating the N-SnapTag-XTEN-CRABP2-H⁶—C, SR protein construct (SEQ ID NO: 3) from these PCR reactions matches that of cloning the R protein construct in the previous section. The N-SnapTag-XTEN-CRABP2-H⁶—C, SR protein construct is listed as SEQ ID NO: 3, and has a Formula of C1640H2584N4460490S11, a molecular weight of 36741.91 Da and C280 of 40825 M⁻¹cm⁻¹.

SnapTag-CRABP2 (SR) Sequence (SnapTag-XTEN Linker-CRABP2-H6)
(SEQ ID NO: 3)
DKDCEMKRTTLDSPLGKLELSGCEQGLHEIIFLGKGTSAADAVEVPAPAAVLGGPEPLMQA
TAWLNAYFHQPEAIEEFPVPALHHPVFQQESFTRQVLWKLLKVVKFGEVISYSHLAALAGN
PAATAAVKTALSGNPVPILIPCHRVVQGDLDVGGYEGGLAVKEWLLAHEGHRLGKPGLGS
GSETPGTSESAPNFSGNWKIIRSENFEELLKVLGVNVMLRKIAVAAASKPAVEIKQEGDTFY
IKTSTTVRTTEINFKVGEEFEEQTVDGRPCKSLVKWESENKMVCEQKLLKGEGPKTSWTRE
LTNDG ELILTMTADDVVCTRVYVREHHHHHH

### Example 11

### Expression and Purification of R, S, C, SR, SS, and CS.

5 μL of Kanamycin (stocked at 50 mg/mL) was added to a culture tube of 5 mL 2×YT media and then inoculated with a stab of either the CRABP2 (R), SnapTag (S), Cutinase (C), SnapTag-CRABP2 (SR), SnapTag-SnapTag (SS) (SEQ ID NO: 4), or cutinase-SnapTag (CS) T7 SHuffle Express cell lines. The culture was then incubated at 30° C. with shaking at 250 RPM for 16 hours. The C, S, and CS cell lines are reported in a previous study.¹ The cultures were then added to a 2 L baffled flask with 500 mL of 2×YT media with 500 μL of Kanamycin (stocked at 50 mg/mL) and shaken at 30° C. in an Innova 44R (New Brunswick Scientific) incubator for 4.5-5 hours (until the OD600 reached ˜0.8). The cultures were then cooled to −20° C. in an ice water bath and then induced with IPTG (2.5 mM final concentration). The induced cultures were shaken for about 1-50 hours, about 6-20 hours, about 10-18 hours, about 12-18 hours, or about 18 hours (about 18 hours) at 20° C. The bacteria were harvested the next day by centrifugation (5000 rpm for 10 min) and the pellet was reconstituted in 1×PBS with DNase. The cells were lysed by sonication on an ice bath in 10 second increments over 3 minutes. The resulting bacterial lysate was centrifuged (11000 rpm for 20 min) to remove cellular debris. The lysate was applied to a 50 mL Kontes Flex column (Kimbal Kontes Glassware) containing 3 mL of Talon IMAC resin that was preequilibrated with 1×PBS buffer. This column was placed on a rotating shaker at 4° C. for 2 hrs. After this period, the supernatant was drained from the column using gravity and the column washed with 1× PBS buffer twice. Weakly bound proteins were first washed off of the resin using a low concentration elution buffer (2×10 mL, 10 mM imidazole, 0.02% NaN3, 1×PBS pH 7.4 @25° C.). The bound protein was then eluted from the resin using elution buffer (3×8 mL, 150 mM imidazole, 0.02% NaN3, 1×PBS pH 7.4 @25° C.). The eluate was then concentrated to 2 mL in a 15 mL Microcon 10 kDa Centrifugal Filter Unit (Millipore) and subsequently purified by size exclusion chromatography (SEC) via an Akta FPLC, on a Hi-Load 16/60 Superdex 200 (GE Healthcare) column using 1×PBS and 0.02% NaN3, pH 7.4 @4° C. as the running buffer. Pure fractions were determined by SDS-PAGE, pooled together, and stocked. The SnapTag-SnapTag sequence is listed as SEQ ID NO: 4 as shown herein below, and has a Formula of C1833H2855N4950526S11, a molecular weight of 40595.49 Da, and ε280 of 42315 M⁻¹ cm⁻¹.

SnapTag-SnapTag Sequence (Start-SnapTag-XTEN Linker-CRABP2-H6)
(SEQ ID NO: 4)
MDKDCEMKRTTLDSPLGKLELSGCEQGLHEIIFLGKGTSAADAVEVPAPAAVLGGPEPLMQ
ATAWLNAYFHQPEAIEEFPVPALHHPVFQQESFTRQVLWKLLKVVKFGEVISYSHLAALAG
NPAATAAVKTALSGNPVPILIPCHRVVQGDLDVGGYEGGLAVKEWLLAHEGHRLGKPGLG
SGSETPGTSESADKDCEMKRTTLDSPLGKLELSGCEQGLHEIIFLGKGTSAADAVEVPAPAA
VLGGPEPLMQATAWLNAYFHQPEAIEEFPVPALHHPVFQQESFTRQVLWKLLKVVKFGEVI
SYSLAALAGNPAATAAVKTALSGNPVPILIPCHRVVQGDLDVGGYEGGLAVKEWLLAHEG
HRLG KPGLGHHHHHH

### Example 12

### CRABP2-10 Conjugate Crystallization.

CRABP2 protein (1000 nmol, 100 μM) was reacted with inhibitor 10 (500 μM, 5 equivalents) in PBS for about 1-50 hours, about 6-20 hours, about 10-18 hours, about 12-16 hours, or about 16 hours. The protein-inhibitor adduct was then purified using SEC and the fractions containing the adduct were pooled, dialyzed, and concentrated to 12 mg/mL (1.275 mL total) using a Centrifugal Filter Unit. The concentrated protein-inhibitor adduct was transferred to a Crystal Gryphon liquid-handling robot (Art Robbins Instruments) and crystallization screening conditions were tested in trays by vapor diffusion at 22° C. over 2-3 weeks with the best conditions including pH 6.5 with varying molecular weights of PEG, as well as in conditions with formate and thiocyanate salts without buffer. After single crystals formed, the crystals were transferred to nylon loops and frozen in liquid nitrogen in preparation for diffraction analysis. X-ray diffraction data were collected using the Life Sciences Collaborative Access Team (LS-CAT) beamline 21-ID-D, —F at the Advanced Photon Source, Argonne National Laboratory.

### Example 13

Data Processing, Structure Determination and Model Refinement. Crystals grew as extensive clusters of needles and required mechanical manipulation to liberate single crystals suitable for data collection. These crystals were looped directly from the mother liquor and flash frozen in liquid nitrogen with no cryoprotectant added. Diffraction data were measured at the Life Sciences Collaborative Access beamlines at Sector 21 of the Advanced Photon Source at Argonne National Laboratory, indexed, integrated and scaled with HKL2000.⁴ All subsequent work was conducted using the CCP4 suite of programs.⁵ The space group and unit cell parameters were confirmed with Pointless.⁶ The structure was determined by molecular replacement with PhaserMR, using a single monomer of Human Cellular Retinoic Acid Binding Protein II (CRABPII) (PDB: 6HKR) with ligands and water molecules removed as the initial phasing model. Two molecules were found in the asymmetric unit. The initial molecular replacement solution was refined with Refmac.^{7,8} The inhibitor molecule and parameter files for refinement were created using GRADE and added to the structure along with 200 water molecules. An N-terminal extension was identified in the maps of one of the two monomers in the asymmetric unit that corresponds to part of a purification tag. The model was improved via successive rounds of manual model building in Coot⁹ and subsequent refinement in Refmac until values of Rwork/Rfree converged.

### Example 14

### Sodium Dodecyl Sulfate-Polyacrylamide Gel Electrophoresis (SDS-PAGE).

All protein samples were diluted in 1×PBS to 10 μM before analysis. 2 μL of the protein sample was mixed with 14 μL of reducing Laemmli buffer. 14 μL of the sample was loaded into a 15-well, 4-15% Tris-glycine precast SDS-PAGE (Biorad) and ran at a constant 150 V with 343 mA for 50 minutes. The gel was then either first imaged for fluorescence on a UV-transilluminator or directly stained using Coomassie-R-250.

### Example 15

### Orthogonality and Selectivity of Enzyme-Inhibitor Pairs.

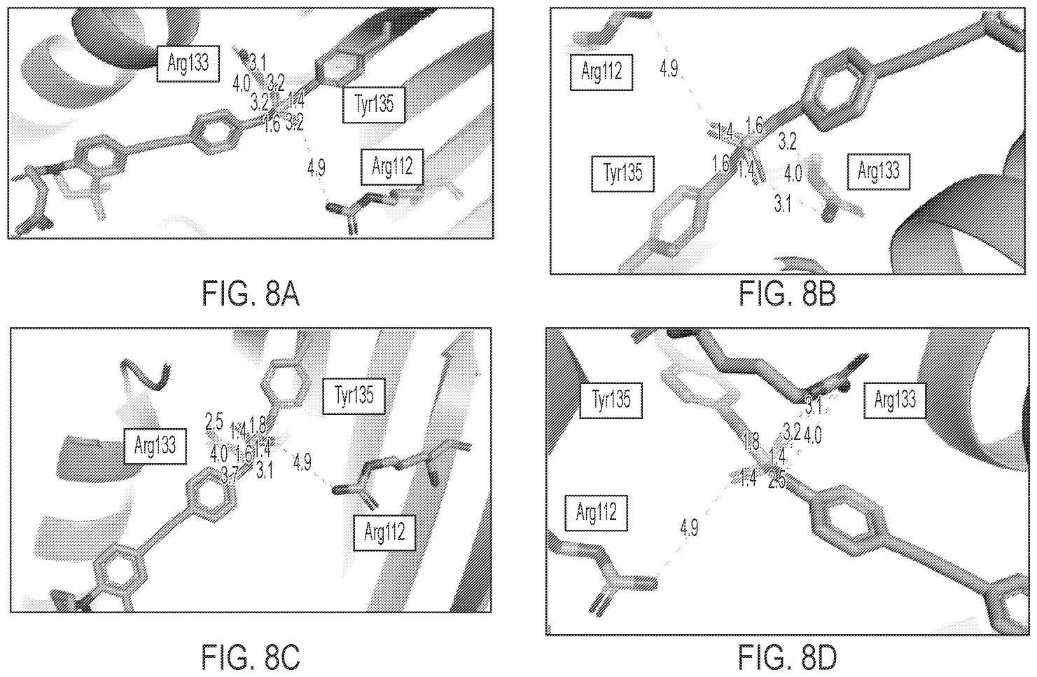

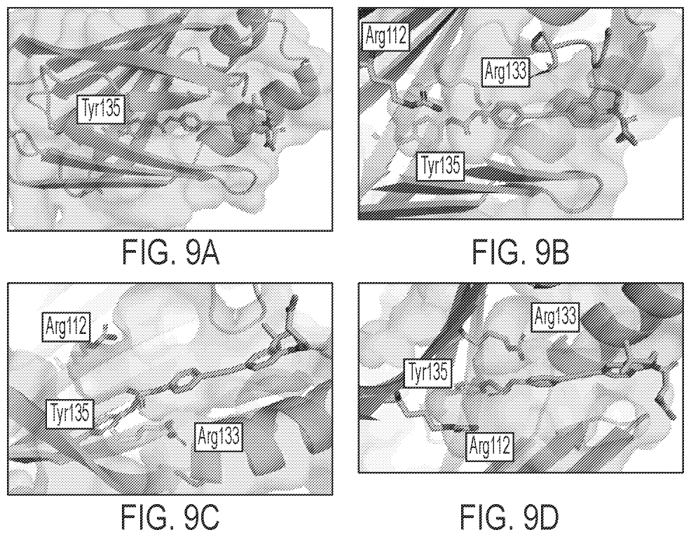

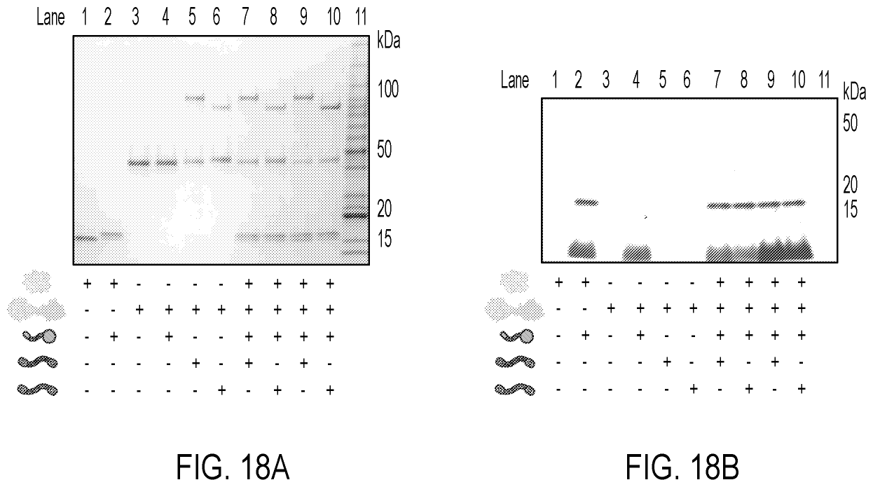

The orthogonality of the CrabTag enzyme-inhibitor system was examined against our existing two chemistries, cutinase and SnapTag. Incubating the CrabTag protein (10 μM in PBS, 25° C.) with either the di-Cutinase or a di-SnapTag linker (1 equiv.) yielded no covalent modification of the CrabTag domain as shown in FIGS. 8 and 9. We next tested the cross-reactivity of SnapTag and cutinase protein domains with the fluorescent CrabTag inhibitor. We incubated a previously reported fusion protein containing SnapTag and cutinase domains (referred to as CS) at 10 μM with the fluorescent CrabTag linker (1 equiv. at 25° C. in PBS). We then added either the di-Cutinase or di-SnapTag linker (1 equiv.) and reacted for about 1-50 hours, about 6-20 hours, about 10-18 hours, about 12-16 hours, or about 16 hours, and the next day completed the reaction sequence with the addition of the CrabTag protein (1 equiv.) to test the reactivity with the previously incubated CrabTag linker as shown in FIG. 18. All reactions were imaged by SDS-PAGE using both fluorescence and Coomassie stain. The CrabTag linker maintained selective reactivity with the CrabTag enzyme, indicating the context-specific reactivity of CrabTag with the covalent retinoid inhibitor.

### Example 16

### Quantification of Reaction Kinetics for CRABP2 Modification and Stability.

We reacted 1 μM of the CrabTag protein domain with varying concentrations (0, 2, 5, 10, 20, 40, 65, and 100 μM) of the fluorescent CrabTag heterobifunctional PEG11 linker in 1×PBS. We tracked the reaction over a time scale of 2 minutes and then allowed the reaction to proceed for about 1-50 hours, about 6-20 hours, about 10-18 hours, about 12-16 hours, or about 16 hours to obtain a final time point for comparison, making the assumption that the reaction was complete on the second day. We obtained time points for by denaturing the sample with reducing Laemmli buffer and ran (SDS-PAGE) to image the reaction progress. We added 5 μL of the reaction mixture to 10 μL of Laemmli buffer for the SDS-PAGE. To determine the relative covalent occupancy of the CrabTag protein domain for each time point to the completed reaction time point, we took images of the gel bands by fluorescence. We used ImageJ to then quantify both the gel bands average weight and mean fluorescence intensity based on the relative fraction of covalently modified protein at discrete time points. The normalization was based on a control band, in which 0 μM of linker was added to give a normalized low value, and the completed reaction time point for each linker concentration, to give the high value. These normalized fluorescence intensities were then fit using GraphPad prism based on a single-phase exponential decay model to obtain observed rate constants, k_{obs}. The second order rate constant was then determined by plotting k_{obs} against the concentrations of fluorescent linker, which gave a Michaelis-Menten kinetic model that included the equilibrium dissociation constant of the linker (KI) and the catalytic rate constant of inactivation (ki). Before testing the stability of the covalent modification, we first removed the excess fluorescent linker from the reaction by size-exclusion chromatography, pooled the fluorescent protein fractions, and concentrated the modified protein to 1 μM. We maintained the protein sample at room temperature for the study. Each day, over a two-week period, we took a 5 μL sample of the protein and mixed it with 10 μL of reducing Laemmli buffer to take a time point. At the completion of the two-weeks, we ran an SDS-PAGE and compared the fluorescence of each time point to a control sample that was pure CrabTag protein without a fluorescent linker. We normalized the fluorescence of the gel and plotted the normalized covalent modification over time. All quantified SDS-PAGE studies were performed in triplicate.

### Example 17

### One-Pot Reaction Progress Tracking and Quantification.

A single, one-pot reaction was designed that has a total of 120 possible reaction permutations in which five reactions occur together in a particular order to form one desired product (16). The goal was to identify the single, most likely pathway by which the desired product was formed. The one-pot reaction was set to 500 μL of a 1×PBS sample containing a previously reported¹ heterotrifunctional SnapTag-SnapTag-cutinase PEG11 linker (8 μM), the cutinase protein (10 μM), the SnapTag-CrabTag fusion protein (20 μM), and the fluorescent CrabTag linker (40 μM). To begin, the cutinase and SnapTag-CrabTag proteins were first mixed, and a control time point was taken for the mixture. The reaction began on the addition of the two linkers and the progress was tracked over time for two hours using denaturing Laemmli buffer and SDS-PAGE. At the completion of the reaction, the gel was imaged by both fluorescence and by staining with Coomassie. The gel images were analyzed and quantified in ImageJ to determine the relative fraction of each species at every time point. The fluorescent gel aided in quantifying intermediate 12 (approximate MW 38 kDa) from intermediate 13 (approximate MW 40 kDa) and the brightfield/stained gel helped to quantify the depletion of the cutinase starting protein. Because the intermediate 15 (approximate MW 76 kDa) and the product 16 (approximate MW 100 kDa) have two fluorescent linkers attached, we divided the mean fluorescence intensities of the corresponding species fluorescent SDS-PAGE protein bands by two. The normalized relative fractions of species were plotted in GraphPad prism. Conversions to relative species concentrations were made by using reaction stoichiometry from the normalized relative fractions from the proposed reaction network. These concentrations were plotted in units of μM using both GraphPad prism and MATLAB for comparison to the model and simulation results for the parameter fitting of the reaction network.

### Modeling and Simulation of One-Pot Reaction Progress.

MATLAB application SimBiology was used for the model diagram creation, simulation, and parameter fitting of the observed concentration profiles for the proposed reaction network. The proposed combined two reaction mechanisms (of the possible 120) is described as follows (equations 1-5):

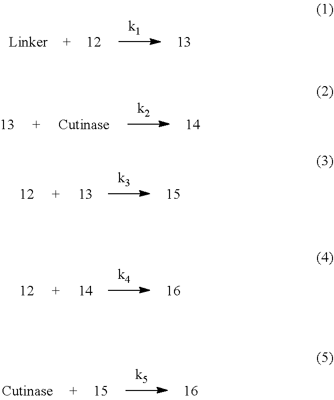

We assumed that because of the constant-volume batch reaction, we could divide the volume of the reaction from the mass-action kinetic equations. We also assumed that the concentration of trifunctional linker was directly proportional to the concentration of intermediate 12 in the rate law expression. The proposed mass-action kinetics and boundary conditions of the reaction network are as follows (equations 6-11):

$$( 6 )$$

$$\frac{{dC}_{Cutinase}}{dt} = - {k}_{2} {C}_{Cutinase} {C}_{13} - {k}_{5} {C}_{Cutinase} {C}_{15}$$

$${[ {C}_{Cutinase} ]}_{t = 0} = 10 μ M$$

$$\frac{{dC}_{12}}{dt} = - {k}_{1} {C}_{12} {C}_{12} - {k}_{3} {C}_{12} {C}_{15} - {k}_{4} {C}_{12} {C}_{14} \qquad ( 7 )$$

$${[ {C}_{12} ]}_{t = 0} = 20 μ M$$

$$\frac{{dC}_{13}}{dt} = {k}_{1} {C}_{12} {C}_{12} - {k}_{2} {C}_{Cutinase} {C}_{13} - {k}_{3} {C}_{12} {C}_{13} \qquad ( 8 )$$

$${[ {C}_{13} ]}_{t = 0} = 0 μ M$$

$$\frac{{dC}_{14}}{dt} = {k}_{2} {C}_{Cutinase} {C}_{13} - {k}_{4} {C}_{12} {C}_{14} \qquad ( 9 )$$

$${[ {C}_{14} ]}_{t = 0} = 0 μ M$$

$$\frac{{dC}_{15}}{dt} = {k}_{3} {C}_{12} {C}_{13} + {k}_{5} {C}_{Cutinase} {C}_{15} \qquad ( 10 )$$

$${[ {C}_{15} ]}_{t = 0} = 0 μ M$$

$$\frac{{dC}_{16}}{dt} = {k}_{4} {C}_{12} {C}_{14} + {k}_{5} {C}_{Cutinase} {C}_{15} \qquad ( 11 )$$

$${[ {C}_{16} ]}_{t = 0} = 0 μ M$$

Using these rate law equations, initial conditions, and boundary condition of the constant-volume reaction vessel, we could simultaneously solve the model network. We used a non-linear regression (lsqnonlin) for statistical modeling with a constant error model, yielding a constant value error model with a=0.9171. The lower boundary for all parameters was 1E-10 and the upper boundary was 100. The system of equations was solved with an ODE15s with 1000 iterations. The estimated parameters were then used in a sensitivity simulation with 10% error and the same parameter values were obtained by convergence. The statistical estimates and data for these parameters are provided in FIGS. 13-15 and Tables 2-5.

### Electrospray Ionization Liquid Chromatography Mass Spectrometry (ESI-MS).

Megamolecules were prepared for LC-MS analysis and evaluation using ESI-MS. Reaction time points were taken by quenching the sample with 0.1% formic acid. The preparation involved the desalting and dilution of the multi-protein assembly to 1 μM in nuclease free water. LC-MS analysis was performed on an Agilent 1200 series HPLC connected to an Agilent 6210A time-of-flight (TOF) mass spectrometer. A 7.5 μL injection of each sample was captured on a C18 trap column (Waters) and eluted using a gradient from 5% to 95% acetonitrile and 0.1% formic acid in water with a flow rate of 0.25 mL/min. Spectra data were analyzed with Agilent MassHunter Qualitative Analysis software to reveal m/z data. These m/z data were deconvoluted using a maximum entropy deconvolution calculation to give the deconvoluted mass spectra.

### Matrix Assisted Lasor Desorption-Ionization Mass Spectrometry (MALDI-MS).

Chemical species in the synthesis of the target inhibitor were evaluated for their molecular weight using MALDI-MS. 1 μL of a chemical solution was added to 4 μL of matrix (15 mg/mL THAP in acetone) and the mixture was spotted on a 384-spot MALDI plate. The spots were allowed to dry thoroughly. The spots were analyzed using an AB SCIEX 5800 MALTI-TOF mass spectrometer. Spectra were acquired in reflector positive mode with an acceleration voltage of 20 kV. M/z values were obtained and reported with hydrogen (H), sodium (Na), or potassium (K) ions.

### Safety Statement.

All research performed in the present disclosure was done so with careful consideration of any risks that are inherent to the materials, instruments, and experiments performed. All research safety guidelines and considerations as provided by the safety data sheets (SDS) and university guides were adhered to for the duration of the research in the present disclosure.

This work establishes a new reaction pair for assembly of megamolecules. The CrabTag domain efficiently reacts with 10 due to the high affinity of the synthetic retinoid and the rapid covalent reaction of the arylfluorosulfate electrophile with the tyrosine nucleophile.^{8,12} The CrabTag protein domain is monomeric, easily expressed in E. coli due to its small size (17 kDa), and is also significant because it is a human-derived enzyme.^{8,22} Further, this inhibition reaction has an effective rate constant of approximately 3,600^{M-1s-1} and the enzyme-inhibitor complex is stable (>95% bound) at room temperature for over two weeks. Finally, the orthogonality of this reaction relative to the other two enzyme-inhibitor reactions used here allowed us to perform single pot assemblies of complex products, demonstrating its utility for convergent megamolecule assembly.

The inventors believe that this new reaction will play a significant role in the assembly of therapeutic antibody mimics,²³ including antibody-drug conjugates,^{24,25} multi-specific antibodies,^{26,27} and hypervalent protein dendrimers.^{28,29} In each of these applications, it is critical that the assembly of several fragments results in a homogeneous target structure without the necessity for intermediate protecting and deprotecting reactions. Further, it will be important that the enzymes are human-derived to minimize unwanted immunogenicity.³⁰⁻³² The inventors expect that having a modular toolbox with the several, orthogonal enzyme-inhibitor reactions described in this work will enable efficient, one-pot assembly of a broad range of megamolecule architectures.

The inventors developed a synthetic retinoid covalent inhibitor that targets the nucleophilic tyrosine residue in cellular retinoic acid binding protein II (CRABP2) in mild conditions. The inventors integrated this enzyme-inhibitor pair into the megamolecule assembly strategy to construct protein scaffolds in one-pot reactions. Here, three orthogonal enzyme-inhibitor reactions occurred simultaneously to form a homogeneous product without the use of protecting groups.

Other types of megamolecules can also be synthesized similarly. Those megamolecules can comprise multiple multi-functional linkers (discussed herein above), fluorescent linkers, multiple fusion proteins, and multiple bio-matters. The megamolecule can further comprise one or more fluorophores or fluorescent tag which can enable the megamolecule for imaging and diagnostic applications and purposes. The multi-functional linkers can have 2-6 chain branches/arms, such as 2 or 3 chain branches/arms. The synthetic inhibitors can be any of a CrabTag inhibitor of 4-(6-((4-((fluorosulfonyl)oxy)phenyl)ethynyl)-4,4-dimethyl-3,4-dihydroquinolin-1(2H)-yl)-4-oxobutanoic acid (Formula 10), a p-nitrophenyl phosphonate (pNPP) inhibitor, and a chloro-pyrimidine (CP) inhibitor. The bio-matters can be any of the proteins, enzymes, antibody fragments and peptides. The enzymes can be any of corresponding CrabTag, SnapTag and cutinase. The bio-matters can further be fusion protein construction of any combination of CrabTag, SnapTag and cutinase, such as CrabTag-Crabtag, CrabTag-SnapTag, CrabTag-cutinase, SnapTag-SnapTag, SnapTag-cutinase, and cutinase-cutinase. Non-limiting examples of additional megamolecules can include the following Configuration 16, Configuration 18, Configuration 19, Configuration 30, Configuration 31, Configuration 32, and Configuration 33, as shown in FIG. 20. The central grey portion represent the multi-functional linker, the red portion represents the CRABP2 (CrabTag), the blue portion represents the cutinase, the yellow portion represents the SnapTag, and the green portion represents a fluorescein tag.

Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the present disclosure. In some example embodiments, well-known processes, well-known device structures, and well-known technologies are not described in detail. Equivalent changes, modifications and variations of some embodiments, materials, compositions, and methods can be made within the scope of the present technology, with substantially similar results.

The following non-limiting discussion of terminology is provided with respect to the present technology. The headings (such as “Introduction” and “Summary”) and sub-headings used herein are intended only for general organization of topics within the present disclosure and are not intended to limit the present disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

As used herein the term “comprising” or “comprises” is used in reference to compositions, methods, and respective component(s) thereof, that are essential to the invention, yet open to the inclusion of unspecified elements, whether essential or not. Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application.

As used herein the term “consisting essentially of refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

“A” and “an” as used herein indicate “at least one” of the item is present; a plurality of such items may be present, when possible.

“About” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters. As used herein, the term “about” when used in connection with a value may refer to ±10% variation from the value.

Other than in the operating examples, or where otherwise indicated, all numbers expressing quantities of ingredients or reaction conditions used herein should be understood as modified in all instances by the term “about.”

As used herein, “about,” “approximately,” “essentially” and “substantially” are understood to refer to numbers in a range of numerals, for example the range of −10% to +10% of the referenced number, or −5% to +5% of the referenced number, or −1% to +1% of the referenced number, or −0.1% to +0.1% of the referenced number.

As used herein, the term “substantially no,” “essentially free” or “substantially free” as used in reference to a particular component may mean that any of the component present constitutes less than 10% by weight, such as less than 9%, less than 8%, less than 7%, less than 6%, less than 5%, less than 4%, less than 3%, less than 2%, less than 1%, less than 0.5% or less than 0.1% by weight.

As used herein, and unless specified otherwise the term “partially” refers to a range of more than 0% and lower than 100%.

As used herein, the term “room temperature” may refer to a temperature in a range of 25° C.±5° C., or 25° C.±3° C.

As used herein, the term “substantially unchanged” by a process (e.g., reacting or heating) refers to a change in value of a characteristic of less than 20%. In embodiments, “substantially unchanged” refers to a change in value of a characteristic of less than 20%, less than 10%, less than 5%, less than 1%, less than 0.5%, or less than 0.1% relative to the value of the characteristic before the process.

As used herein, the term “and/or” includes any and all combinations of one or more of the associated listed items.

As used herein, the term “between” in the context of a range is inclusive of the two ends of the range, unless specified otherwise.

The abbreviation, “e.g.” or “i.e.” are used herein to indicate a non-limiting example. Thus, the abbreviation “e.g.” or “i.e.” is synonymous with the term “for example.” Where used herein, the terms “example” and “such as,” particularly when followed by a listing of terms, are merely exemplary and illustrative and should not be deemed to be exclusive or comprehensive.

What is claimed is:

1. A synthetic inhibitor comprising:
a backbone comprising a unit (—R₁—) selected from the group of diphenylacetylene (Formula A), 4-(phenyl)ethynyl)-4,4-dimethyl-3,4-dihydroquinolin (Formula B), 4,4-dimethyl-6-(phenylethynyl)-1,2,3,4-tetrahydroquinoline (Formula C), phenyl acetylene (Formula D), a compound represented by Formula E, and a compound represented by Formula F;
a first functional group (—R_{f1}) provided at a first end of the backbone; and
a second functional group (—R_{f2}) provided at a second end of the backbone, the second functional group (—R_{f2}) comprising one of a carboxylic acid, —CO—CH₂CH₂—COOH, —CO—R₆—COOH, an amide, —CO—R₆—CO—NH—(PEG)ₘ-, an activated carboxylic acid, a n-hydroxysuccinimide (NHS) ester, an alcohol, a primary amine, a secondary amine, a fluorophore or fluorescein tag, a click chemistry molecule selected from the group of bicyclononyne (BCN), trans-cyclooctene (TCO), tetrazine (Tz), dibenzocyclooctyne (DBCO) and azide, and a combination thereof, wherein the —R₆— can be an aryl group or an alkyl having 1 to 6 carbon atoms, and m is in a range of 1-50,
wherein the first functional group is specific for irreversibly inhibiting a bio-matter, and 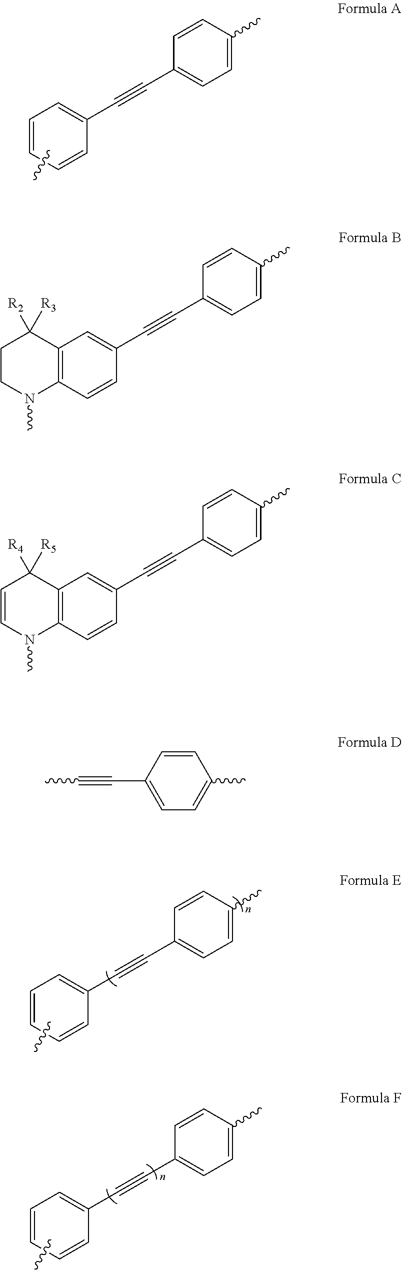
wherein each of R₂, R₃, R₄ and R₅ is independently selected from the group of an alkyl having 1 to 6 carbons, a fluoro-alkyl having 1 to 6 carbons, and an aryl, [symbol] represents a bond to the first functional group (—R_{f1}) or the second functional group (—R_{f2}), and n is in a range of 1 to 10.

2. The synthetic inhibitor of claim 1, wherein the bio-matter comprises a protein, an enzyme, an antibody fragment, or a peptide.

3. The synthetic inhibitor of claim 1 wherein the first functional group (—R_{f1}) comprises a fluorosulfate group (F—SO₂—O—), an arylfluorosulfate group (F—SO₂—O—Ar), an arylsulfonyl fluoride group (F—SO₂—Ar), or a sulfonyl fluoride group (F—SO₂—), a p-nitrophenyl phosphonate, an O⁶-benzylguanine, an α-haloalkane, a haloaromatic compound, a beta-lactam, an aglycone, a hydroxamic acid-benzophenone, a cognate oriT oligonucleotide sequence, a cysteine-reactive ATP-binding site inhibitor, a quinone methide, an α-halo phosphonic acid, a formylchromone, a cognate RNA sequence, adenosine, cytosine, a cognate DNA nicking site, a thiirane, a hydroxamic acid, an α-ketoxazole inhibitor, an electrophilic steroid, a phosphonate, a carbamate, an aromatic alkyne, beloranib, and a combination of the foregoing.

4. The synthetic inhibitor of claim 1, wherein the first functional group comprises a fluorosulfate (F—SO₂—O—), an arylfluorosulfate (F—SO₂—O—Ar), an arylsulfonyl fluoride (F—SO₂—Ar) or a sulfonyl fluoride (F—SO₂—).

5. The synthetic inhibitor of claim 1, wherein the first functional group comprises a fluorosulfate (F—SO₂—O—) or an sulfonyl fluoride (F—SO₂—).

6. The synthetic inhibitor of claim 1, wherein the unit (—R₁—) is selected from the group of diphenylacetylene backbone (Formula A), 4-(phenyl)ethynyl)-4,4-dimethyl-3,4-dihydroquinolin backbone (Formula B), and 4,4-dimethyl-6-(phenylethynyl)-1,2,3,4-tetrahydroquinoline (Formula C).

7. The synthetic inhibitor of claim 1, wherein the second functional group comprises the carboxylic acid, the amide, the primary amine, the secondary amine, or the click chemistry molecule selected from the group of bicyclononyne (BCN), trans-cyclooctene (TCO), tetrazine (Tz), dibenzocyclooctyne (DBCO) and azide.

8. The synthetic inhibitor of claim 1, wherein the synthetic inhibitor comprises a compound having a structure according to Formula 10, Formula 11, and Formula J to Formula O: 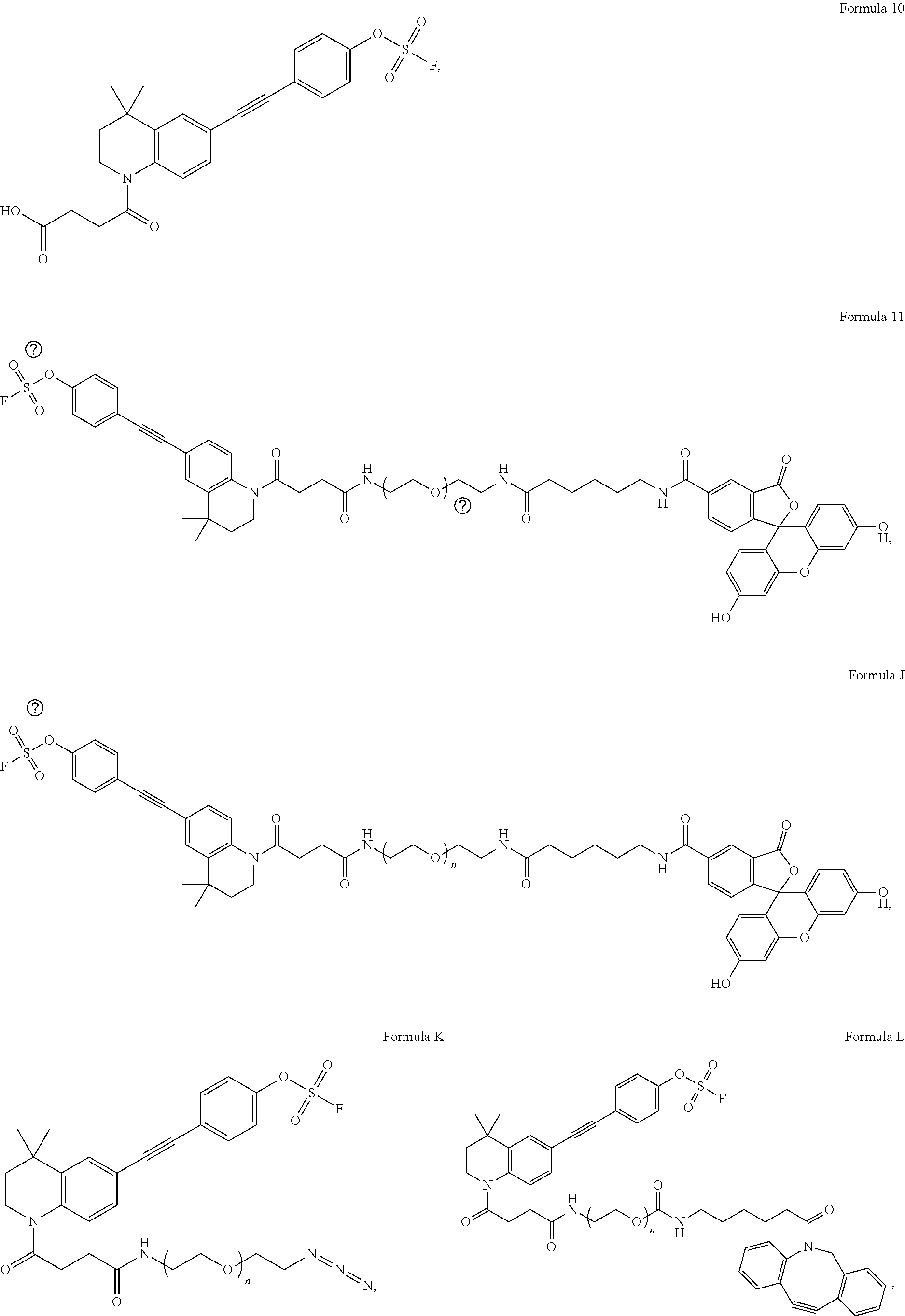 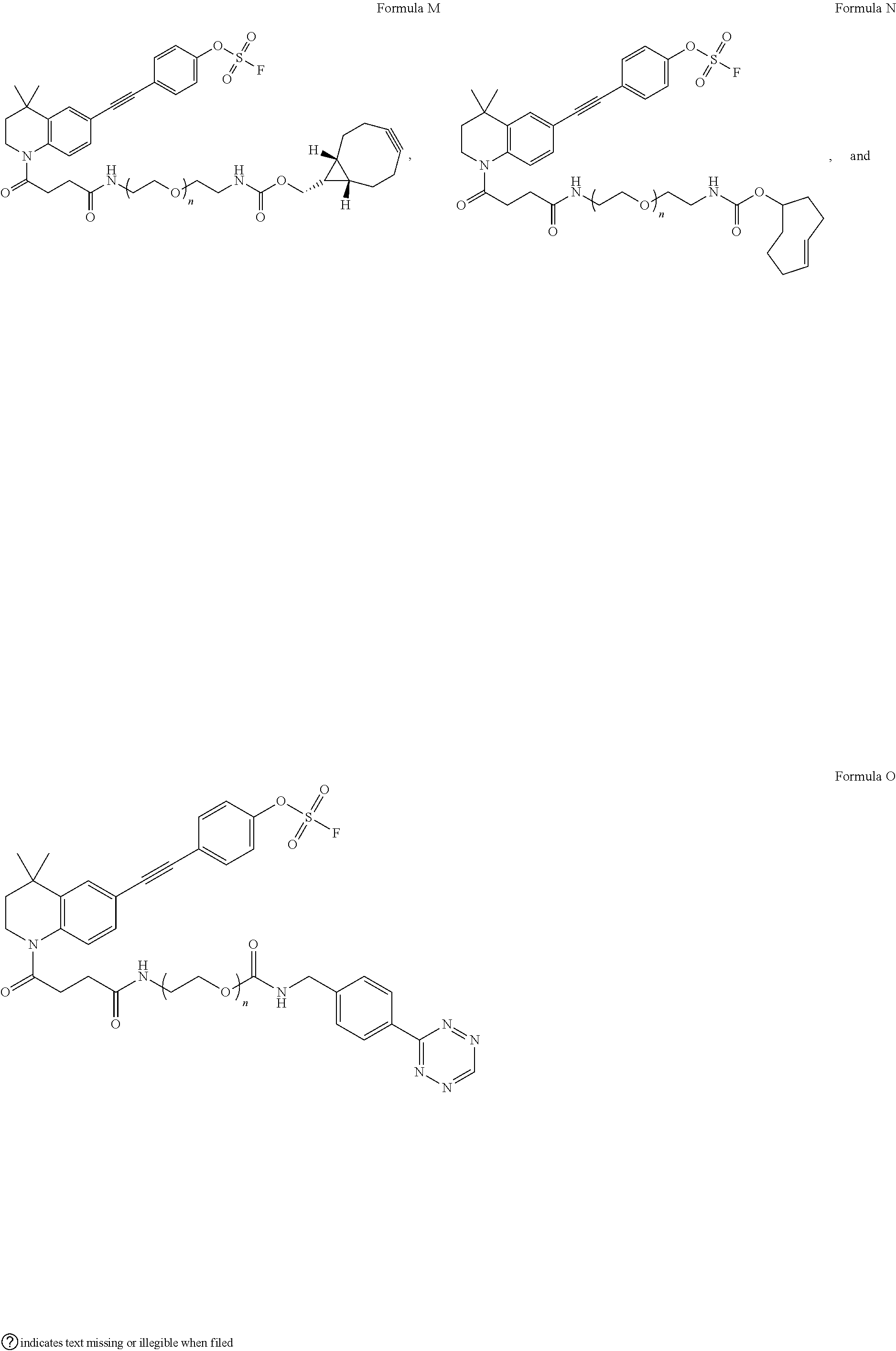
wherein n is in a range of 1-50.

9. The synthetic inhibitor of claim 1, wherein the synthetic inhibitor comprises the compound having the structure according to Formula 10.

10. A multi-functional linker comprising:
a central compound having a core and two chain branches; and
two inhibitors, each of which comprises a first functional group (—R_{f1}) specific for irreversibly inhibiting a bio-matter, and a second functional group (—R_{f2}),
wherein each of the second functional groups (—Re) independently comprises a carboxylic acid, —CO—CH₂CH₂—COOH, —CO—R₆—COOH, an amide, —CO—R₆—CO—NH—(PEG)ₘ-, an activated carboxylic acid, a n-hydroxysuccinimide (NHS) ester, an alcohol, a primary amine, a secondary amine, a fluorophore or fluorescein tag, a click chemistry molecule selected from the group of bicyclononyne (BCN), trans-cyclooctene (TCO), tetrazine (Tz), dibenzocyclooctyne (DBCO) and azide, and a combination thereof, wherein the —R₆— can be an aryl or an alkyl having 1 to 6 carbons, and m is in a range of 1-50.

11. The multi-functional linker of claim 10, wherein each bio-matter independently comprises a protein, an enzyme, an antibody fragment, or a peptide.

12-22. (canceled)

23. The multi-functional linker of claim 10, wherein each of the at least two synthetic inhibitors are independently selected from the group of an inhibitor having a structure according to Formula 10, Formula 11 and Formula J to Formula O, a p-nitrophenyl phosphonate (pNPP) inhibitor, a chloro-pyrimidine (CP) inhibitor, a haloalkane inhibitor, a benzylcytosine inhibitor, and a benzylguanine inhibitor: 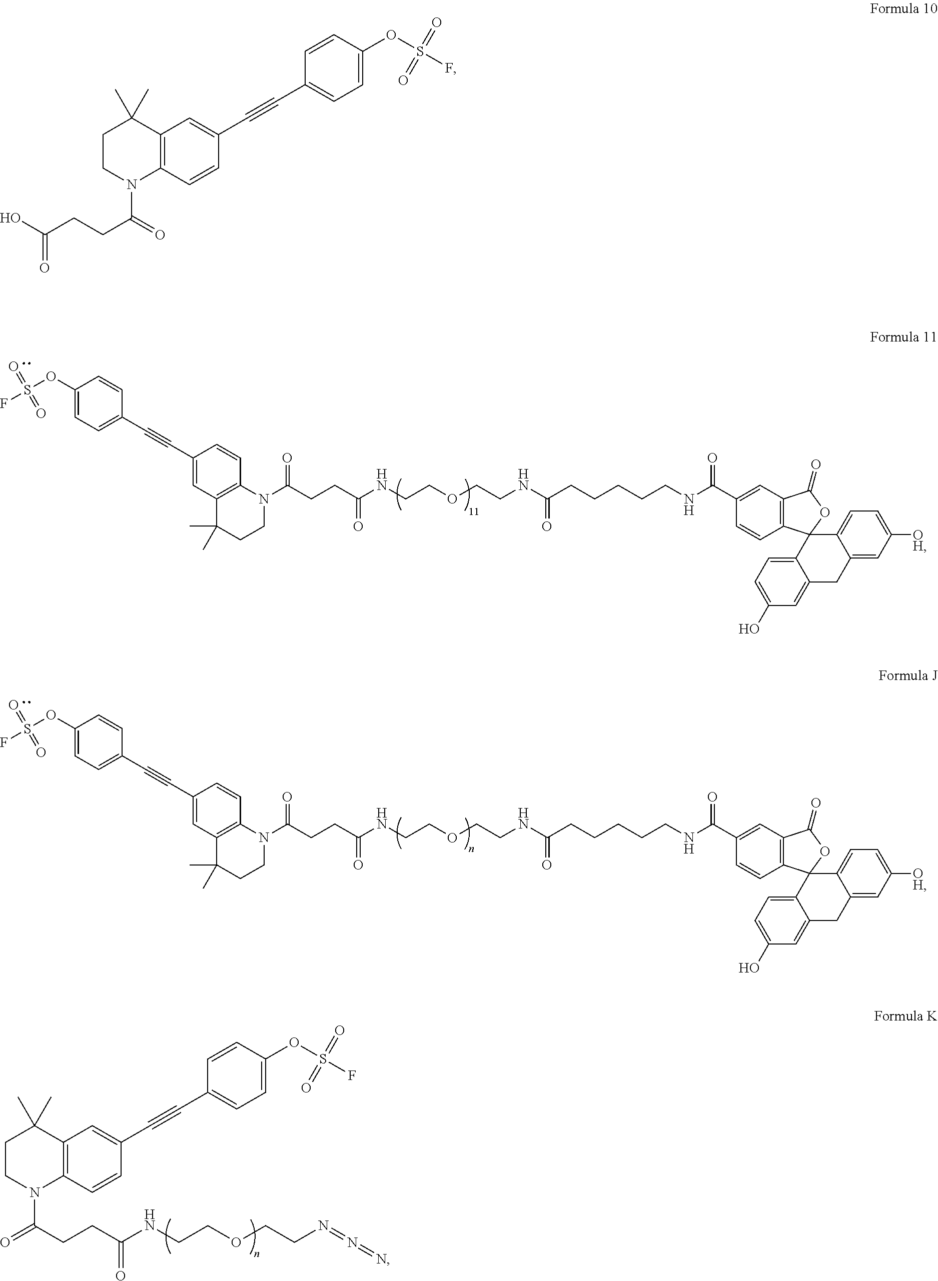 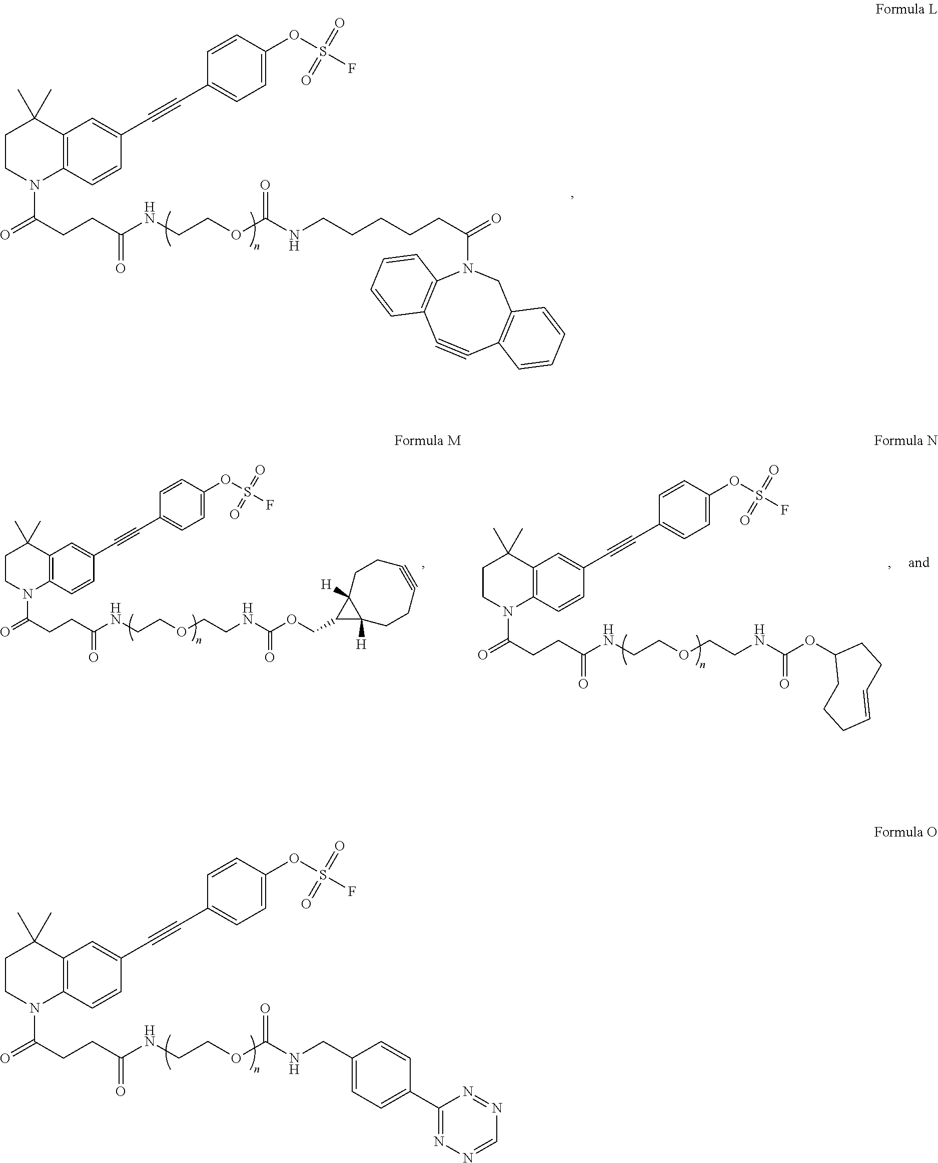
wherein n is in a range of 1-50.

24-25. (canceled)

26. The multi-functional linker of claim 10, having a structure according to Formula 17,

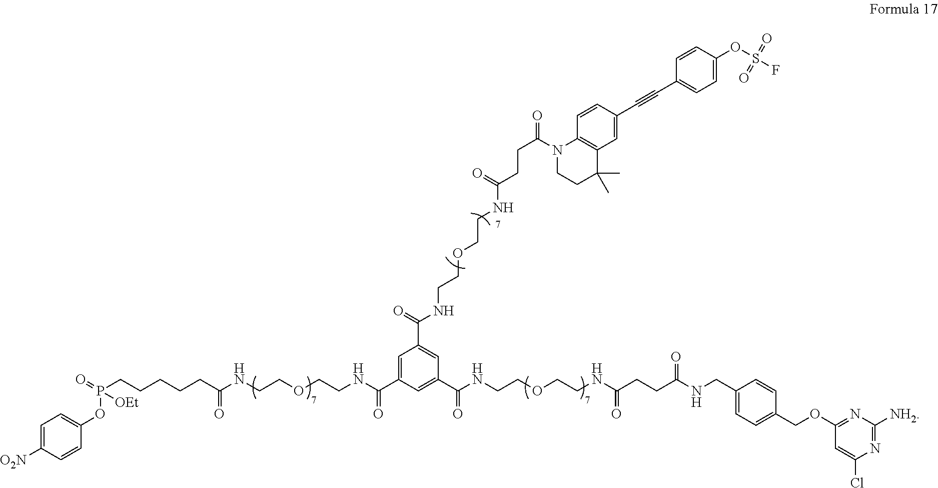

27. (canceled)

28. A multi-functional linker comprising:
a central compound having a core, a first chain branch extending outward from the core and having a first end distal from the core, and a second chain branch extending outward from the core and having a second end distal to the core;
a first inhibitor comprising a first functional group specific for irreversibly inhibiting a first bio-matter, and the first inhibitor provided at the first end of the first chain branch; and
a second inhibitor comprising a second functional group specific for irreversibly inhibiting a second bio-matter, and the second inhibitor provided at the second end of the second chain branch.

29-50. (canceled)

51. A megamolecule comprising:
a first bio-matter and a second bio-matter, and
a multi-functional linker comprising:
a central compound comprising a core, a first chain branch extending from the core and having a first end distal from the central compound, and a second chain branch extending from the core and having a second end distal from the central compound;
a first inhibitor having a first functional group specific for irreversibly inhibiting the first bio-matter, and provided at the first end of the first chain branch; and
a second inhibitor having a second functional group specific for irreversibly inhibiting the second bio-matter, and provided at the second end of the second chain branch,
wherein the first bio-matter is coupled to the first inhibitor,
the second bio-matter is coupled to the second inhibitor,
and the megamolecule has a molecular weight in a range of about 1.0 KDa to about 10.0 MDa.

52-77. (canceled)

78. A method of preparing a synthetic inhibitor having a structure according to Formula 10, 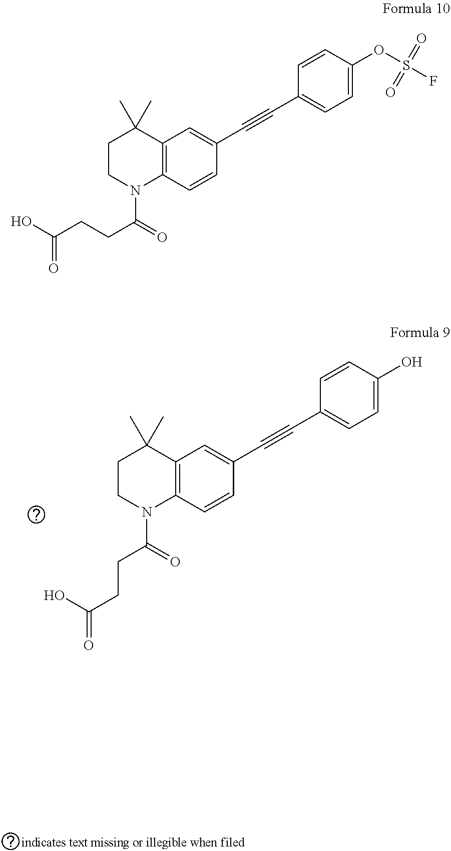 [SMILES: CC1(C)CCN(C(=O)CCC(=O)O)c2ccc(C#Cc3ccc(OS(=O)(=O)F)cc3)cc21 ; CC1(C)CCN(C(=O)CCC(=O)O)c2ccc(C#Cc3ccc(O)cc3)cc21]
the method comprising: reacting a compound having a structure of Formula 9 (4-(6-((4-hydroxyphenyl)ethynyl)-4,4-dimethyl-3,4-dihydroquinolin-1(2H)-yl)-4-oxobutanoic acid) with [4-(acetylamino)phenyl]imidodisulfuryl difluoride (AISF) in a first solvent to form a first product mixture comprising the synthetic inhibitor having the structure according to Formula 10.

79-126. (canceled)

127. A method for preparing a multi-functional linker of any of claims 10-50, the method comprising:
reacting a central compound with a first inhibitor and a second inhibitor,
wherein the central compound includes a core, a first chain branch extending outward from the core and a second chain branch extending outward from the core,
wherein the first chain branch has a first end group distal from the core, and the second chain branch has a second end group distal from the core,
wherein the first inhibitor comprises a first functional group specific for irreversibly inhibiting a first bio-matter, and a fourth functional group,
wherein the second inhibitor comprises a second functional group specific for irreversibly inhibiting a second bio-matter, and a fifth functional group,
wherein the reacting couples the first end group with the fourth functional group,
wherein the reacting couples the second end group with the fifth functional group, and
wherein the first and second functional groups are each independently selected from the group of a fluorosulfate (F—SO₂—O—), an arylfluorosulfate (F—SO₂—O—Ar), a sulfonyl fluoride (F—SO₂—), an arylsulfonyl fluoride (F—SO₂—Ar), a p-nitrophenyl phosphonate (pNPP), a chloro-pyrimidine (CP), a haloalkane, a benzylcytosine, and a benzylguanine.

128-142. (canceled)

143. A method for preparing a megamolecule, the method comprising:
reacting a multi-functional linker according to any of claims 10-50 with a first bio-matter and a second bio-matter to form the megamolecule,
wherein the first bio-matter has a first active enzyme including a first terminus, and the second bio-matter has a second active enzyme including a second terminus,
wherein the multi-functional linker comprises a central compound, a first synthetic inhibitor and a second synthetic inhibitor each coupled to the central compound,
wherein the first synthetic inhibitor comprises a first functional group specific for irreversibly inhibiting the first reactive enzyme at the first terminus,
wherein the second synthetic inhibitor comprises a second functional group specific for irreversibly inhibiting the second reactive enzyme at the second terminus,
wherein the reacting comprises irreversibly inhibiting the first active enzyme at the first terminus by the first synthetic inhibitor to couple the first bio-matter to the first synthetic inhibitor,
wherein the reacting comprises irreversibly inhibiting the second active enzyme at the second terminus by the second synthetic inhibitor to couple the second bio-matter to the second synthetic inhibitor,
wherein the first and second functional groups are each independently selected from the group of a fluorosulfate (F—SO₂—O—), an arylfluorosulfate (F—SO₂—O—Ar), a sulfonyl fluoride (F—SO₂—), an arylsulfonyl fluoride (F—SO₂—Ar), a p-nitrophenyl phosphonate (pNPP), a chloro-pyrimidine (CP), a haloalkane, a benzylcytosine and a benzylguanine group, and
wherein the megamolecule has a molecular weight in a range of about 1.0 KDa to about 10.0 MDa.

144-172. (canceled)

173. A megamolecule prepared according to the method of claim 143.

174. A method of preparing a synthetic inhibitor having a structure of Formula 11: 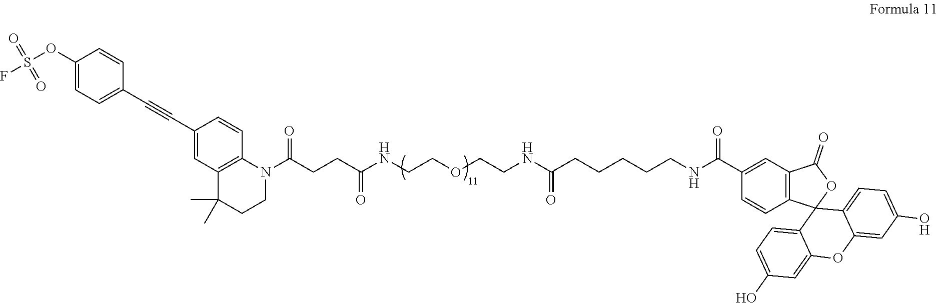
the method comprising:
admixing a synthetic inhibitor have a structure according to Formula 10: 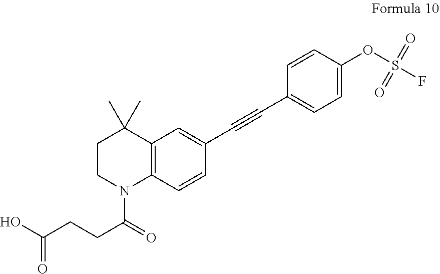 [SMILES: CC1(C)CCN(C(=O)CCC(=O)O)c2ccc(C#Cc3ccc(OS(=O)(=O)F)cc3)cc21]
with 6-[fluorescein-5(6)-carboxamido]hexanoic acid in 4-(4,6-dimethoxy-1,3,5-triazin-2-yl)-4-methyl-morpholinium chloride (DMTMM) and tetrahydrofuran (THF);
adding N-Methylmorpholine (NMM) and stirring for about 0.5-24 hours, or about 1-10 hours, or about 2-6 hours, or about 3-5 hours, or about 3.5-4.5 hours, or about 4 hours;
removing DMTMM, THE and N-Methylmorpholine (NMM) from the container;
adding dimethylformamide (DMF), azido-PEG11-amine and N-Methylmorpholine (NMM) to the container;
stirring for about 6-24 hours, or about 8-20 hours, or about 12-16 hours, or about 16 hours to form a solution;
diluting the solution with 9:1 dimethyl sulfoxide (DMSO):H₂O;
purifying the solution on a C18 reversed phase column via HPLC using TFA as the mobile phase additive to form a liquid mixture comprising the synthetic inhibitor having the structure of Formula 11; and
lyophilizing the liquid mixture to yield the synthetic inhibitor having the structure of Formula 11.
